(19) United States
(12) Patent Application Publication
(10) Pub. No.: US 2025/0313886 A1
(43) Pub. Date: Oct. 9, 2025

(54) SYSTEMS AND METHODS FOR ALLELE ENRICHMENT USING MULTIPLEXED BLOCKER DISPLACEMENT AMPLIFICATION

(71) Applicant: William Marsh Rice University, Houston, TX (US)

(72) Inventors: David Yu ZHANG, Houston, TX (US); Ping SONG, Houston, TX (US); Xi CHEN, Houston, TX (US)

(73) Assignee: William Marsh Rice University, Houston, TX (US)

(21) Appl. No.: 19/057,316

(22) Filed: Feb. 19, 2025

Related U.S. Application Data
(63) Continuation of application No. 16/971,411, filed on Aug. 20, 2020, now Pat. No. 12,331,350.
(60) Provisional application No. 62/632,712, filed on Feb. 20, 2018.
(60) Provisional application No. 62/649,138, filed on Mar. 28, 2018.

(51) Int. Cl. C12Q 1/6858

(52) U.S. Cl. CPC C12Q 1/6858

(57) ABSTRACT

Provided herein are reagents and methods for simultaneously enriching many potential rare genetic variants at different genetic loci. The rare variants enriched can include single nucleotide polymorphisms (SNPs), single nucleotide variants, or small insertions and deletions. Embodiments of the invention include procedures for integration with downstream next generation sequencing (NGS) analysis. Embodiments of the invention include analysis of nonpathogenic SNPs for the determination of cell identity and detection of cell contamination using qPCR or NGS.

### REFERENCE TO RELATED APPLICATIONS

The present application is a continuation of U.S. application Ser. No. 16/971,411, filed Aug. 20, 2020, which is a national phase application under 35 U.S.C. § 371 of International Application No. PCT/US2019/018690, filed Feb. 20, 2019, which claims the priority benefit of U.S. provisional application No. 62/632,712, filed Feb. 20, 2018, and U.S. provisional application No. 62/649,138, filed Mar. 28, 2018, the entire contents of each of which are incorporated herein by reference.

### STATEMENT REGARDING FEDERALLY SPONSORED RESEARCH

This invention was made with government support under Grant No. RO1 CA203964 awarded by the National Institutes of Health. The government has certain rights in the invention.

### REFERENCE TO A SEQUENCE LISTING

This application contains a Sequence Listing XML, which has been submitted electronically and is hereby incorporated by reference in its entirety. Said Sequence Listing XML, created on Feb. 19, 2025, is named RICEP0042USC1.xml and is 355,508 bytes in size.

### BACKGROUND

### 1. Field

The present invention relates generally to the field of molecular biology. More particularly, it concerns compositions and methods for multiplex enrichment of many different sequence variations having low VAFs.

### 2. Description of Related Art

Sequence variations in genomic DNA include nonpathogenic single nucleotide polymorphisms (SNPs) that can collectively distinguish individuals from each other, pathogenic germline mutations that can cause or increase the likelihood of genetic diseases, and pathogenic somatic mutations that cause cancer. The technical difficulty distinguishing these sequence variations depends strongly on both the fraction of the DNA that contains the variation (the variant allele fraction; VAF) and the number of variations that need to be simultaneously profiled.

For profiling a few sequence variations (<5) at relatively high VAF (e.g., 5%), quantitative PCR is the standard approach that is used for many FDA-approved or cleared diagnostic tests. For profiling many (1000+) sequence variations at relatively high VAF, microarrays or low-depth next-generation sequencing (NGS) is the commercially preferred method. For profiling a few sequence variations at very low VAF (e.g., <0.1%), digital droplet PCR and ultradeep NGS with unique molecular barcodes are being developed. However, simultaneously profiling many sequence variations each at potentially low VAF remains a significant challenge because microarrays lack the sensitivity for low VAFs, digital PCR cannot be multiplexed past a very small number, and ultradeep NGS is slow and cost prohibitive when applied to many potential mutations.

### SUMMARY

Provided herein are reagents and methods to simultaneously enrich, by 100-fold or more, many different sequence variations having low VAFs. For example, sequence variations originally at 0.1% VAF may be enriched to 10% VAF or higher, allowing profiling via low-depth NGS or microarrays in highly multiplexed settings. Applications of these methods include detection of cell line contamination and analysis of rare cancer mutations in liquid biopsy settings.

In one embodiment, provided herein are methods for simultaneously amplifying allelic variants at least ten genetic loci, the method comprising: (a) mixing a sample comprising DNA with a DNA polymerase and a blocker displacement amplification (BDA) oligo set for each genetic locus, each BDA oligo set comprising (i) a BDA forward primer, (ii) a BDA blocker, and (iii) a BDA reverse primer, wherein at least four nucleotides at the 3′ end of each BDA forward primer sequence are also present at or near the 5′ end of its respective BDA blocker sequence, wherein each BDA blocker contains a 3′ sequence or modification that prevents extension by DNA polymerase, and wherein the concentration of each BDA blocker is at least twice that of its respective BDA forward primer; and (b) subjecting the mixture to at least four cycles of amplification, thereby producing amplicons. In some aspects, the methods simultaneously amplify allelic variants at between ten and 1,000,000 genetic loci. In some aspects, the DNA comprises an allelic variant at at least one of the genetic loci.

In some aspects, the final concentrations of all BDA forward primers in the mixture sum to more than 50 nanomolar and less than 50 micromolar. In some aspects, each cycle of amplification in step (b) comprises: (i) a denaturation step at a temperature between 75° C. and 105° C. for between 1 second and 300 seconds; and (ii) an anneal step at a temperature between 45° C. and 75° C. for between 15 seconds and 3 hours. In some aspects, the DNA polymerase is a high-fidelity DNA polymerase, such as, for example, Phusion, NEB Q5, or Kapa HiFi. In certain aspects, the DNA polymerase has 3′ to 5′ exonuclease activity. In certain aspects, each BDA blocker has a 3′ modification that prevents 3′ to 5′ exonuclease activity. In certain aspects, the 3′ modification that prevents 3′ to 5′ exonuclease activity comprises inverted DNA nucleotides, a phosphorothioate backbone, one or more carbon spacers, or one or more polyethylene glycol (PEG) spacers. In some aspects, step (a) further comprises mixing an intercalating dye that selectively fluoresces when bound to double-stranded DNA, such as, for example, a SybrGreen, EvaGreen, or Syto dye.

In some aspects, the methods further comprise (c) selecting the amplicons produced by step (b) by size. In certain aspects, the selection is performed using affinity beads, affinity columns, gel electrophoresis, or capillary electrophoresis.

In some aspects, the methods further comprise (d1) amplifying the size-selected amplicons by polymerase chain reaction using primers having next-generation sequencing (NGS) adapters and/or sample index sequences, thereby producing adapter and/or sample index modified amplicons. In some aspects, the methods further comprise (d2) ligating onto both ends of the size-selected amplicons oligonucleotides having next-generation sequencing (NGS) adapters and/or sample index sequences, thereby producing adapter and/or sample index modified amplicons.

In some aspects, the methods further comprise (e) performing next-generation sequencing of the adapter and/or sample index modified amplicons.

In some aspects, the concentration of each BDA reverse primer is determined based on a reads analysis of a previous calibration NGS experiment, wherein the concentration of each BDA reverse primer is increased relative to the concentration used for the previous calibration NGS experiment. In certain aspects, the concentration of each BDA reverse primer follows a formula: [rP]new=[rP]old*(Reads_median/Reads_amplicon)\hat{ }X, where [rP]old is the previous concentration of the reverse primer, Reads_median is the median reads mapped to each amplicon, Reads_amplicon is the reads mapped to the amplicon corresponding to said reverse primer, and X is an adjustment factor between 0.25 and 1.

In some aspects, the concentration of each BDA forward primer is determined based on a reads analysis of a previous calibration NGS experiment, wherein the concentration of each BDA forward primer is increased relative to the concentration used for the previous calibration NGS experiment. In certain aspects, the concentration of each BDA forward primer follows a formula: [fP]new=[fP]old*(Reads_median/Reads_amplicon)\hat{ }X, where [fP]old is the previous concentration of the forward primer, Reads_median is the median reads mapped to each amplicon, Reads_amplicon is the reads mapped to the amplicon corresponding to said forward primer, and X is an adjustment factor between 0.25 and 1.

In one embodiment, provided herein are methods for designing the sequences of BDA oligo sets, each comprising a BDA forward primer, a BDA blocker, and a BDA reverse primer, for a locus group of interest, the method comprising: (1) selecting either the (+) or (−) DNA strand to be used as a BDA template for the locus group of interest; (2) removing loci that require incompatible enrichment regions; (3) creating a list of candidate BDA forward primers, BDA blockers, and BDA reverse primers for each remaining locus; (4) selecting a random BDA forward primer, BDA blocker, and BDA reverse primer from the candidate list for each locus; (5) evaluating the likelihood of primer dimer formation for the set of all selected BDA forward primers, BDA blockers, and BDA reverse primers; (6) replacing with other candidate sequences from (3) some BDA forward primers, BDA blockers, or BDA reverse primers identified in step (5) as forming primer dimers; and (7) repeating steps (5) and (6) for a fixed number of cycles, or until the evaluation in step (6) returns an acceptable result.

In some aspects, the BDA oligonucleotide sets are for use in simultaneously amplifying allelic variants at multiple genomic loci. In some aspects, evaluating in step (5) comprises evaluating the potential reverse complementarity between the 3′-most 4-8 nucleotides of all possible pairs of BDA forward primers, BDA blockers, and BDA reverse primers. In some aspects, evaluating in step (5) comprises evaluating the potential reverse complementarity between any continuous subsequences 6-10 nucleotides in length of all possible pairs of BDA forward primers, BDA blockers, and BDA reverse primers.

In one embodiment, provided herein are methods for analyzing NGS reads generated by a method of the present embodiments, the method comprising: (a) removing read sequences having a quality below a set quality threshold; (b) aligning the remaining read sequences to the expected wildtype amplicon sequences; (c) identifying each variation in read sequences that differ from the corresponding wildtype amplicon sequence in an enrichment region; (d) calculating the fraction of read sequences aligned to each amplicon that correspond to each variation; and (e) discarding reads corresponding to variations in which the calculated fraction is below a set threshold value.

In some aspects, the threshold value in step (e) is between 0.1% and 10%. In some aspects, the methods further comprise calculating a variant allele fraction (VAF) for each variation not discarded in step (e) by using the formula of VAF=RF/(E*(1−RF)+RF), where E is the expected fold-enrichment of the variation and RF is the observed reads fraction of the variation. In certain aspects, the value of E for some variants is determined based on calibration experiments using reference samples bearing said variants at known VAFs. In certain aspects, the value of E for some variants is determined based on the nucleotide identities of the wildtype sequence, the variant sequence, and the sequence located 50 nt upstream and 50 nt downstream of the variant sequence (e.g., based on statistical or machine learning of E values for similar sequences). In certain aspects, the methods further comprise calculating a quantitative estimate of the fraction of the minority cell type from a heterogeneous cell sample by taking a median of the inferred VAF values for 3 or more different variants. In some aspects, the methods further comprise calculating a quantitative estimate of the fraction of the minority cell type from a heterogeneous cell sample by taking a mean of the inferred VAF values for 3 or more different variants.

In one embodiment, provided herein are aqueous solutions of oligonucleotides molecules, the solution comprising at least 10 BDA oligo sets, each BDA oligo set comprising (i) a BDA forward primer, (ii) a BDA blocker, and (iii) a BDA reverse primer, wherein at least four nucleotides at the 3′ end of each BDA forward primer sequence are also present at or near the 5′ end of its corresponding BDA blocker sequence, wherein each BDA blocker contains a 3′ sequence or modification that prevents extension by DNA polymerase, and wherein the concentration of each BDA blocker is at least twice that of its corresponding BDA forward primer, wherein each BDA blocker is complementary to a genomic region bearing a single nucleotide polymorphism (SNP) in which the alternative allele has a population frequency of between 10% and 90%, and wherein each corresponding BDA forward primer is not complementary to the SNP locus. In some aspects, the solution comprises between ten and 1,000,000 BDA oligo sets. In some aspects, none of the BDA forward primers and none of the BDA reverse primers are complementary to any SNP in which the alternative allele has a population frequency of over 1%. In some aspects, the genomic position that each BDA reverse primer binds is located between 100 nt and 500 nt away from the genomic position that its corresponding BDA forward primer binds. In some aspects, the calculated ΔG°'s for each BDA forward primer binding to its corresponding complement are all within 2 kcal/mol of each other at 60° C. in 0.18 M Na+. In some aspects, the calculated ΔG° for each BDA blocker binding to its corresponding complement is between 0.5 kcal/mol and 3.5 kcal/mol more favorable than the ΔG° of binding between the corresponding BDA forward primer and its complement at 60° C. in 0.18 M Na+.

In one embodiment, provided herein are methods for detecting contamination of a base cell line, the method comprising: (a) extracting genomic DNA from a cell sample; (b) mixing the genomic DNA with a DNA polymerase, dNTPs, and the aqueous solution of any one of the present embodiments; (c) subjecting the mixture to at least four cycles of amplification, thereby producing amplicons; and (d) analyzing the amplification reaction or the amplicon mixture. In some aspects, the SNPs are nonpathogenic. In some aspects, the BDA blockers selectively hybridize to the SNP alleles of the base cell line. In some aspects, the BDA blockers do not selectively hybridize to the SNP alleles of the base cell line.

In some aspects, each cycle of amplification in step (c) comprises: (i) a denaturation step at a temperature between 75° C. and 105° C. for between 1 second and 300 seconds; and (ii) an anneal step at a temperature between 45° C. and 75° C. for between 15 seconds and 3 hours. In some aspects, step (b) further comprises mixing the genomic DNA with an intercalating dye that selectively fluoresces when bound to double-stranded DNA. In some aspects, between 10 and 80 cycles of amplification are performed in step (c). In some aspects, step (d) comprising comparing the amplification Cycle Threshold (Ct) value to a reference value.

In some aspects, step (b) further comprises mixing the genomic DNA with an internal control set of primers and a Taqman probe to the internal control, and wherein the reference value is the Taqman probe-derived Ct value of the internal control. In certain aspects, the at least 3 aliquots of the genomic DNA sample are run, and wherein the analysis in step (d) is performed based on the difference between the median intercalating dye Ct value and the median Taqman probe Ct value. In certain aspects, at least 3 aliquots of the genomic DNA sample are run, and wherein the analysis is performed based on the difference between the mean intercalating dye Ct value and the mean Taqman probe Ct value.

In some aspects, step (d) comprises: (i) preparing an NGS library based using the amplicons produced in step (c); (ii) performing high-throughput sequencing of the NGS library to obtain NGS reads; and (iii) interpreting the NGS reads. In certain aspects, the BDA blockers selectively hybridize to the SNP alleles of the base cell line, and wherein a positive result for contamination is obtained if the analysis of the NGS reads indicates the presence of any SNP alleles differing from the base cell sample SNP alleles above a threshold reads fraction. In certain aspects, the threshold reads fraction is between 0.1% and 10%. In certain aspects, the methods further comprise identifying the contaminant based on the pattern of detected SNP alleles that differ from the SNP alleles of the base cell lines. In certain aspects, the BDA blockers do not selectively hybridize to the SNP alleles of the base cell line, and wherein a positive result for contamination is obtained if the analysis of the NGS reads indicates the presence of contaminant SNP alleles above a threshold reads fraction. In certain aspects, the threshold reads fraction is between 0.1% and 10%.

In one embodiment, provided herein are panels of nonpathogenic SNPs comprising at least 30 nonpathogenic SNPs, wherein each SNP has an alternative allele with a population frequency of between 10% and 90%, wherein each pair of SNPs is either on different chromosomes or has a genomic distance of at least 2,000 nucleotides, wherein the sequence 50 nucleotides upstream and 50 nucleotides downstream of the SNP is unique within the organism's genome. In some aspects, the panel is for use in verifying the genomic identity of an individual or an organism. In some aspects, the sequence 50 nucleotides upstream and 50 nucleotides downstream of the SNP are unique within the organism's genome if no other region of the organism's genome has a greater than 90% homology to the sequence. In some aspects, each SNP has an alternative allele with a population frequency of between 20% and 80%. In some aspects, the organism is Homo sapiens. In some aspects, the panels comprise SNPs from each of the 22 pairs of autosomes in the human genome.

In one embodiment, provided herein are methods of preparing the panel of any one of the present embodiments, the method comprising: (a) obtaining a list of candidate SNPs with exact genomic positions and estimates of population frequencies; (b) removing candidate SNPs with alternative alleles having population frequency of below 10% or above 90%; (c) randomly selecting roughly double the number of desired SNPs from the remaining list, wherein the randomly selected SNPs are spaced by at least 2,000 nucleotides from any other randomly selected SNPs located on the same chromosome; (d) removing SNPs where the sequence 50 nucleotides upstream and 50 nucleotides downstream of the SNP exists in duplicate or with high homology to other regions of the genome; and (e) selecting a final list of SNPs for the panel from the remaining candidate SNPs. In some aspects, the methods further comprise preparing a BDA oligonucleotide set for each of the remaining candidate SNPs.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Schematic and mechanism of single-plex blocker displacement amplification (BDA). The sequence of fP is provided in SEQ ID NO: 20. The sequence of B is provided in SEQ ID NO: 100. The sequence of WT (NA18537) is provided in SEQ ID NO: 251. The sequence of Variant (NA18562) is provided in SEQ ID NO: 252.

FIG. 2: Multiplex BDA (mBDA) to simultaneously enrich potential sequence variants at many groups of genetic loci.

FIG. 3: Appending NGS sequencing adapters to mBDA amplicons via PCR.

FIG. 4: Summary of NGS experimental results.

FIGS. 5A-B: Considerations for fP, B, and rP sequence design for mBDA. (FIG. 5A) Examples of potential nonselective binding interactions between fP, B, and/or rP that can lead to primer dimer formation. (FIG. 5B) Subgrouping variants/loci for mBDA.

FIGS. 6A-D: Inferring initial VAF from post-mBDA NGS reads. (FIG. 6A) Inference of VAF from unenriched multiplex PCR library, based on fraction of reads mapped to variant allele at each locus. (FIG. 6B) Relationship between initial VAF and post-mBDA NGS reads fraction mapped to variant allele. (FIG. 6C) Summary of fold enrichment E for different SNP types. (FIG. 6D) Inferred VAF from reads fraction (from post-mBDA NGS) and fitted E values.

FIG. 7: Alternative ligation-based method for appending adapter sequences onto mBDA amplicons.

FIG. 8: Detection of cell line contamination using SNP patterns.

FIG. 9: Patterns of SNPs across 85 SNP loci for 23 individuals (volunteers from the Houston area).

FIG. 10: Cell line contamination detection using 80-plex mBDA and qPCR readout.

FIGS. 11A-E: Detection of minute cell line contamination using multiplex blocker displacement amplification (mBDA). (FIG. 11A) A list of SNPs in which the primary cell line is homozygous (either for the human reference allele, or the minor allele). (FIG. 11B) The Blocker is designed to be perfectly complementary to the DNA template bearing primary SNP allele, and mismatched to the variant SNP allele. (FIG. 11C) A 21-plex mBDA set designed against the primary alleles for all 21 SNP positions selected. (FIG. 11D) mBDA maintains a large Ct difference between the primary and variant alleles in highly multiplexed reactions. (FIG. 11E) Detection of different frequencies of HeLa contamination in NA18537 via mBDA using qPCR.

FIGS. 12A-D: Generalizability of the mBDA approach to detecting arbitrary human cell line contaminants. (FIG. 12A) SNP genotypes for 21 different contaminant samples. (FIG. 12B) Simulated distribution of the number of unique contaminant alleles out of the 21 SNPs observed, against NA18537, based on reported SNP population allele frequencies and assuming independence between SNPs. (FIG. 12C) Simulated distribution of the number of homozygous SNPs out of 80 for an arbitrary cell line, based on reported SNP population allele frequencies and assuming independence between SNPs. (FIG. 12D) Summary of qPCR results for detection of 5% contaminant in NA18537 with 37 different human DNA contaminants.

FIGS. 13A-E: Using mBDA to detect variant DNA sequences with low variant allele frequency (VAF) using low-depth next-generation sequencing (NGS). (FIG. 13A) Summary of the mBDA NGS library preparation workflow. (FIG. 13B) Summary of NGS results on an amplicon library without using mBDA. (FIG. 13C) Summary of NGS results for the mBDA NGS library on the sample 0.1% VAF sample. (FIG. 13D) Summary of the variant read fraction (VRF) for each SNP locus in the libraries described in panels (FIG. 13B) and (FIG. 13C). (FIG. 13E) Reproducibility of the VRFs in two replicate mBDA NGS libraries using the 0.1% VAF sample.

FIGS. 14A-E: Quantitating variant VAFs based on mBDA NGS VRF. (FIG. 14A) Theoretical relationship between VRF and VAF for different allele enrichment-fold (EF). (FIG. 14B) The relationship between (1-VRF)/VRF and (1-VAF)/VAF. (FIG. 14C) Summary of inferred EF for each of the 80 variant SNP alleles using NA18537 as the primary alleles. (FIG. 14D) Relative EF values for different VAF inputs. (FIG. 14E) VAF limit of detection (LoD) for standard amplicon NGS vs. mBDA NGS.

FIGS. 15A-D: Detection and quantitation of DNA contamination using mBDA NGS. (FIG. 15A) Results for 22 mBDA libraries on a single MiSeq chip. (FIG. 15B) Comparison libraries using standard amplicon NGS on the samples contaminated with S9 (HeLa). (FIG. 15C) Summary of variant call accuracy using the 0.019% VAF LoD threshold described in FIG. 14E. (FIG. 15D) Receiver operator characteristic (ROC) plot for variant calls using the data in panel (FIG. 15C).

FIGS. 16A-D: Determination of contaminant identity based on mBDA NGS data. (FIG. 16A) Calculation of contaminant likelihood based on mBDA variant calls. (FIG. 16B) Heatmap plot of log 10(L) values for all pairwise combinations of all 22 tested samples (with contamination fraction between 0.07% and 0.22%) vs. 35 database genotypes, based on data from FIG. 15A. (FIG. 16C) Representative distributions of log 10(L) for 4 different samples. (FIG. 16D) Plot of highest and second-highest log 10(L) values against the contamination fraction.

### DETAILED DESCRIPTION

A typical blocker displacement amplification (BDA) system uses three different oligonucleotides: the forward primer (fP), the blocker (B), and the reverse primer (rP). The forward primer and the reverse primer are designed to function as standard PCR primers. In some embodiments, the binding of the forward and reverse primers to their respective reverse complement sequences have a computed melting temperature of approximately 50° C., approximately 55° C., approximately 60° C., approximately 65° C., or approximately 70° C. in a buffer suitable for PCR, at primer concentrations of between 100 nM and 5 μM. In some embodiments, the binding of the forward and reverse primers to their reverse complement sequences have a computed standard free energy of binding (ΔG°_{fP} and ΔG°_{rP}, respectively) of approximately −11 kcal/mol at approximately 50° C., approximately 55° C., approximately 60° C., approximately 65° C., or approximately 70° C. in a buffer suitable for PCR.

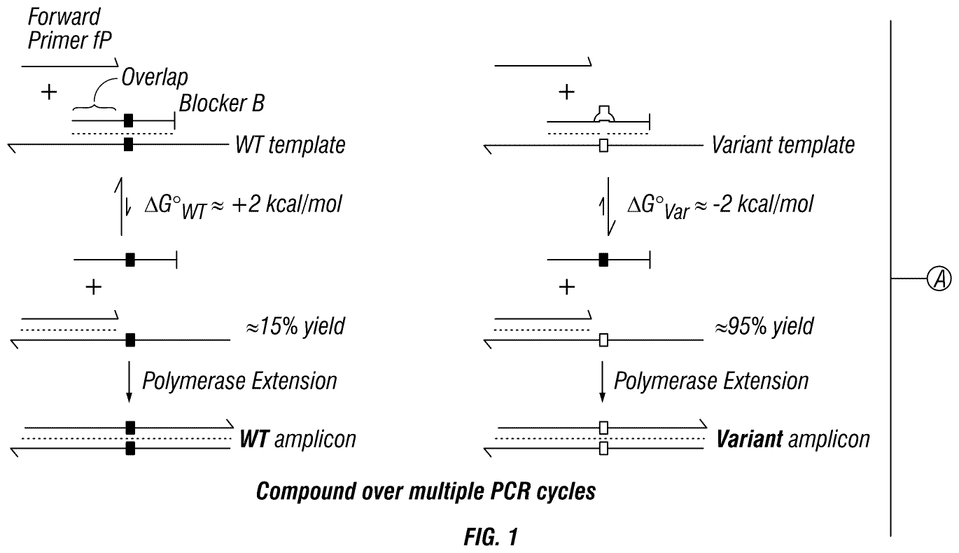

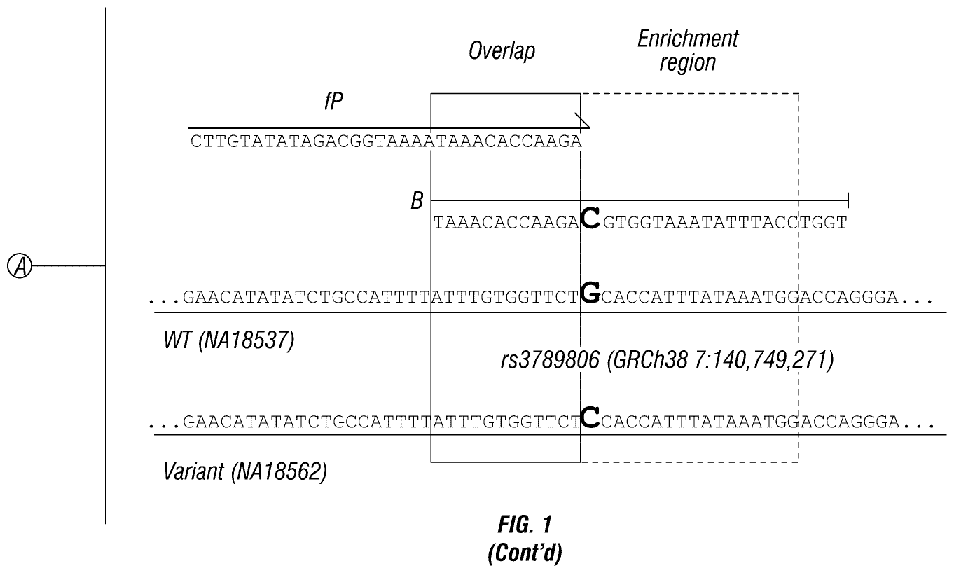

The forward primer (fP) and the blocker (B) are designed to have a certain degree of sequence overlap, with several 3′ most nucleotides of fP being identical to several nucleotides on B near the 5′ end. This forces the binding of fP and the binding of B, to overlapping regions on a template DNA molecule, to be mutually exclusive (FIG. 1). With high probability, a three-stranded molecule comprising the template, fP, and B colocalized via DNA hybridization interactions will rapidly dissociate, releasing either a single-stranded fP or single-stranded B into solution. In some embodiments, the number of nucleotides of overlap between the forward primer and the blocker is 4, 5, 6, 7, 8, 9, 10, 11, 12, 13, 14, 15, 16, 17, 18, 19, or 20. In some embodiments, the standard free energy of binding (ΔG°) of the overlapping nucleotides to their reverse complement sequences is −4 kcal/mol at approximately 50° C., approximately 55° C., approximately 60° C., approximately 65° C., or approximately 70° C. in a buffer suitable for PCR.

In some embodiments, the binding of the blocker to its reverse complement sequence has a computed melting temperature of approximately 55° C., approximately 60° C., approximately 65° C., approximately 70° C., approximately 75° C., or approximately 80° C. in a buffer suitable for PCR, at blocker concentrations of between 100 nM and 5 μM. In some embodiments, the binding of the blocker to its reverse complement sequence has a computed standard free energy of binding (ΔG°_{B}) of approximately −14 kcal/mol at approximately 50° C., approximately 55° C., approximately 60° C., approximately 65° C., or approximately 70° C. in a buffer suitable for PCR.

The blocker (B) is designed to be perfectly complementary to a wildtype sequence, so any template with a variant allele in the enrichment region produces a destabilizing mismatch bubble when B is bound to the template. Consequently, fP will more favorably displace B on variant templates than on wildtype templates, and this results in a difference in the per-cycle amplification yield. The yield difference is compounded across multiple cycles of PCR. The enrichment region typically includes all bases to the 3′ of the overlap region, except for the four 3′-most nucleotides on B. All variants at any position in the enrichment region will be enriched.

In some embodiments, the standard free energy of the blocker binding to its reverse complement (ΔG°_{B}) is stronger than the standard free energy of the forward primer binding to its reverse complement (ΔG°_{fP}) by between −1 kcal/mol and −4 kcal/mol at approximately 50° C., approximately 55° C., approximately 60° C., approximately 65° C., or approximately 70° C. in a buffer suitable for PCR. In some embodiments, the blocker comprises a sequence at or near the 3′ end that does not hybridize to the template and prevents DNA polymerase extension. In some embodiments, the blocker comprises a chemical modification at or near the 3′ end that prevents DNA polymerase extension. In some embodiments, the blocker comprises a chemical modification at or near the 3′ end that prevents 3′->5′ exonuclease activity by error-correct DNA polymerases. In some embodiments, the said chemical modification comprises inverted DNA nucleotides. In some embodiments, the said chemical modification comprises 3-carbon spacers (C3 spacers).

In the design of the present probe system, the ΔG° term denotes the standard free energy of hybridization between two complementary strands. In one instance, the standard free energies of hybridization between regions of the present probe system can be approximately calculated based on a base pair stacking approach. In this method, two adjacent base pairs comprise one stack, which has a defined enthalpy (ΔH°) and entropy (ΔS°) value. The standard free energy of each stack (ΔG°) at a particular temperature τ (in Kelvin) can be calculated from the equation ΔG°=ΔH°−τΔS°. The standard free energies of several stacks can be summed to evaluate the standard free energy of a binding region. The ΔH° and ΔS° values of DNA-DNA stacks can be found in SantaLucia and Hicks (2004). Because current literature-provided standard free energy values are incomplete and of limited accuracy, experimental testing is needed to determine a true value of ΔG° for any two complementary strands, but the literature-guided values provide a rough (typically within 3 kcal/mol or 15%) estimate of the ΔG°.

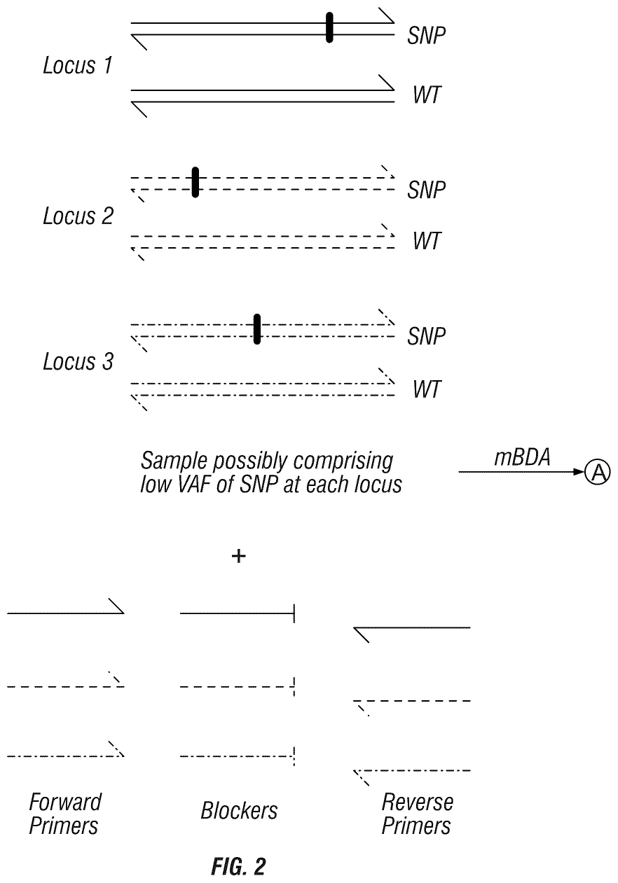

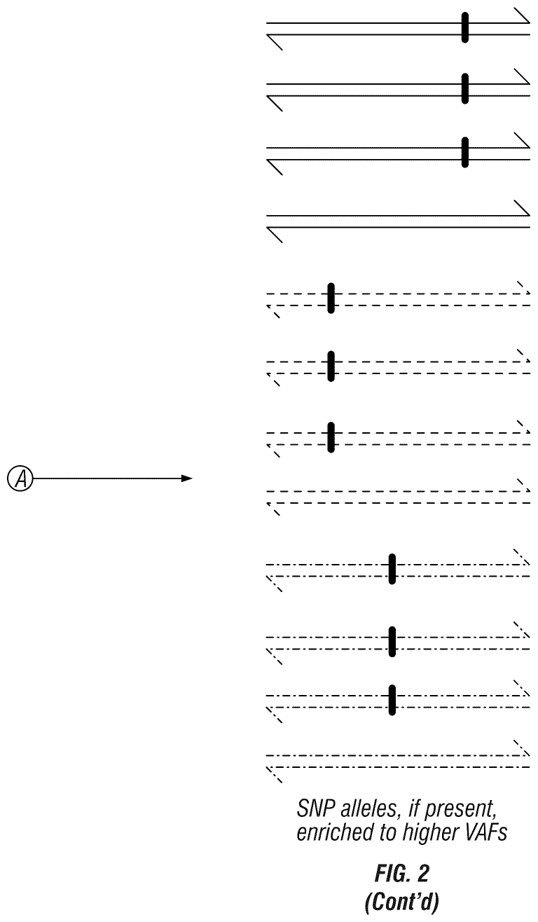

For multiplex BDA (mBDA) to simultaneously enrich potential sequence variants at many groups of genetic loci, different fP, B, and rP species are employed for each BDA system. These are all combined in solution simultaneously with the sample, a DNA polymerase, dNTPs, and buffers amenable for PCR (FIG. 2). To prevent DNA-based inhibition of PCR, the total concentration of all oligo species should be kept under 50 micromolar. The length of the anneal/extend step of the PCR reaction is inversely proportional to the concentration of the lowest of the forward primer species. To prevent excessively long protocols, it is recommended that all fP and rP concentrations be at least 100 picomolar. The concentration of each B species should be at least 2× that of its corresponding fP species. The concentration of each rP species can be adjusted to allow relatively uniform amplification of all BDA amplicons. In some embodiments, the concentration of each rP species is determined based on the observed reads for each BDA amplicon from a prior NGS experiment with known rP concentrations.

### I. DESIGN OF OLIGOS FOR MULTIPLEX BDA

In addition to the standard design principles of single-plex BDA described above, oligo design for multiplex BDA (mBDA) requires further consideration to prevent formation of unintended amplicons from two reverse primers in opposite directions and undesired “primer dimer” species. The first issue can largely be avoided if all BDA systems target the same (+) or (−) strand of template DNA, or alternatively if the template is short (e.g., cell-free DNA from blood plasma, or genomic DNA sheared by ultrasonication or fragmentase).

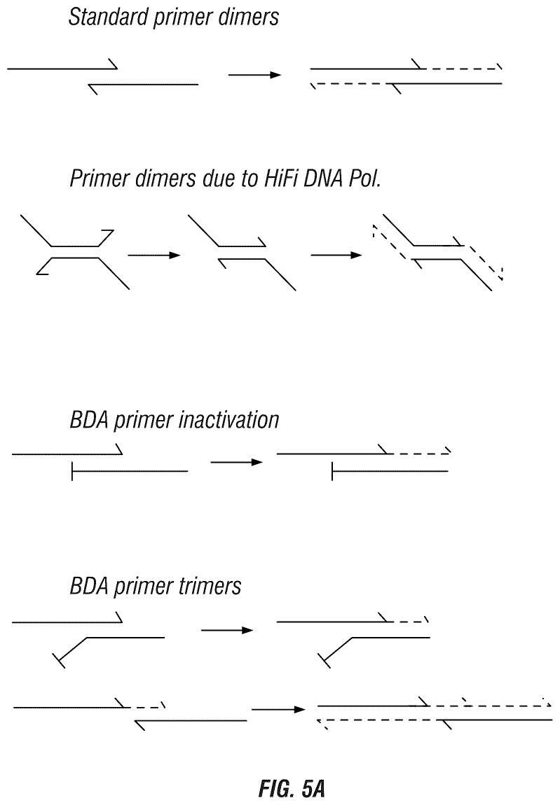

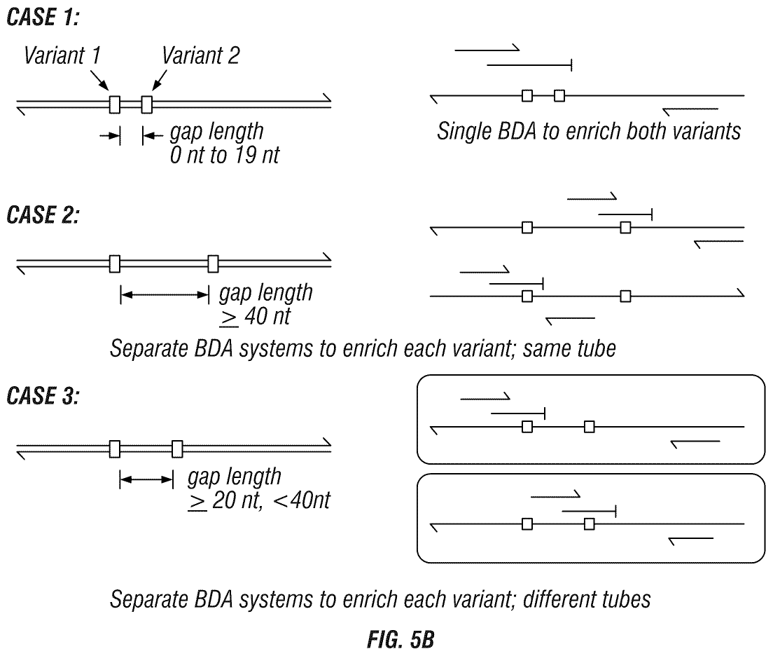

The primer dimer issue is more complex, because the possibility of primer dimer formation increases nonlinearly with the number of different primer and blocker species in solution. For example, in a 10-plex mBDA system, there are 20 primers and 10 blockers, for a total of Combination (30,2)=435 pairwise interactions; for a 20-plex mBDA system, there are 40 primers and 20 blockers, for a total of Combination (60,2)=1,770 pairwise interactions. The complexity of the problem becomes worse because some primer “dimer” species arise from more complex mechanisms involving three different oligo species or more (FIG. 5A). Shown are examples of potential nonselective binding interactions between fP, B, and/or rP that can lead to primer dimer formation. Algorithms for mBDA sequence design should penalize candidate sequence sets when they are predicted to exhibit any of the listed interactions.

One embodiment of an algorithm that designs mBDA primers and blockers to largely avoid primer dimers is described below. Many potential variations of this algorithm should be obvious to those of ordinary skill in the art of nonconvex optimization software.

1. Determine the preferred direction of each mBDA system, in terms of the blocker binding to either the (+) or the (−) strand of biological DNA. The direction preference may be informed (1) by predicted ΔΔG° of the blocker binding to a specific variant vs. the wildtype, (2) by consideration of other compatibility with other loci of interest as briefly described in FIG. 5B, and (3) by the average expected length of the DNA to be analyzed.

2. Partitioning the loci of potential variants into one or more groups, based on the distance between loci of variants as illustrated in FIG. 5B. When the distance is fewer than about 20 nucleotides, a single blocker B can cover both variant loci within its enrichment region (Case 1). When the distance is farther than about 40 nucleotides, two separate BDA systems can be designed to function within the same reaction without expected adverse effects (Case 3). However, when the distance is between about 20 and about 40 nucleotides, there is insufficient room to place a second BDA system, so two separate BDA systems in two separate reactions are needed (Case 2). BDA oligos for enriching different loci within the same group are meant to be used in the same solution. Disjoint potential variations in which each group of ≤20 nt loci are spaced from all other loci by over 100 nt are all compatible with each other and can be placed in the same group. At the other extreme, when potential variations can exist at any position in a very long stretch of DNA, such as in a tumor suppressor gene like TP53, the loci may need to be partitioned into 3 to 5 different groups. The remainder of the mBDA sequence design protocol is performed on fP, B, and rP species within a single group.

3. Creating a list of candidate fP, B, and rP sequences for each BDA system within the group. In some embodiments, fP and B candidate sequences satisfy the following constraints: (1) the fP and rP each binds to the template with a calculated ΔG° between −10 kcal/mol and −15 kcal/mol at the temperature and salinity condition of the anneal cycle of PCR; (2) B binds to the template with a calculated ΔG° between −12 kcal/mol and −18 kcal/mol at the temperature and salinity condition of the anneal cycle of PCR; (3) the portion of fP that does not overlap with B binds to the templates with a calculated ΔG° of between −5.5 kcal/mol and −8.5 kcal/mol at the temperature and salinity condition of the anneal cycle of PCR; (4) the amplicon length is between 60 nt and 300 nt long; and (5) B's enrichment region should cover the loci bearing potential sequence variations. Depending on the number of continuous loci to be enriched, there may be between 1 and 25 different candidate sequences for each of fP and B in each BDA system. Depending on the stringency of the amplicon length, there may be between 10 and 200 candidate sequences for each rP. For example, for a 20-plex BDA, there will be 20 different sets of fP candidates, 20 different sets of B candidates, and 20 different sets of rP candidates.

4. Selecting a random initial set of sequences, the set comprising one randomly selected fP sequence for each BDA system, one randomly selected B sequence for each BDA system, and one randomly selected rP sequence for each BDA system. For example, for a 20-plex BDA with 15 candidates for each fP, B, and rP species, there will be 15⁶⁰≈3.7*10⁷⁰ possible sets of initial random sequences.

5. Performing a heuristic evaluation of the primer dimer likelihood of the randomly selected set of sequences through the calculation of a quantitative “Badness” or “Loss” score that is initialized to 0, and then is incremented based on evaluation of individual oligo properties and/or pairwise oligo interactions. In some embodiments, a pair of oligos in the set contributes to Badness/Loss if the five 3′-most nucleotides of the first oligo are the reverse complement of the five 3′-most nucleotides of the second oligo. In some embodiments, the number of nucleotides at the 3′-most end evaluated for potential reverse complementary with other 3′-most nucleotides is 4, 5, 6, 7, 8, 9, 10, 11, 12, 13, 14, 15, 16, 17, 18, 19, or 20. In some embodiments, a pair of oligos contribute to Badness/Loss based on the calculated standard free energy of interaction ΔG°ᵢ; in some embodiments, the Badness/Loss contribution may be linear, quadratic, or exponential in ΔG°ᵢ. In some embodiments, a pair of oligos contribute to Badness/Loss based on the number of continuous nucleotides of the first strand that are reverse complementary to a number of continuous nucleotides on the second strand. In some embodiments, a single oligo contributes to Badness/Loss based on the calculated free energy of its predicted secondary structure.

6. Creating a new mBDA oligo set based on the existing BDA oligo set, except with one randomly selected fP, B, or rP species replaced by another candidate of the same type.

The Badness/Loss of the new mBDA oligo set is evaluated.

7. Deciding whether to accept the potential sequence change based on the Badness/Loss of the new set, compared to the Badness/Loss of the old sequence set. In some embodiments, the new mBDA oligo set is accepted only if the Badness/Loss is improved over the old set. In the field of computer optimization, this strategy is known as gradient descent or stochastic gradient descent. Alternatively, mBDA oligo sets with slightly worse Badness/Loss are also accepted with some probability inversely proportional to the amount of Badness/Loss change. In some embodiments, this probability diminishes over time. In the field of computer optimization, this strategy is known as simulated annealing. Other methods for nonconvex optimization, such as genetic algorithms, may also be applied.

8. Repeating steps (6) and (7) for a fixed number of cycles, or until the Badness/Loss of the BDA oligo set is below an acceptable threshold.

In some embodiments, the above algorithm is applied with the variation that the fP and B candidate sequences are evaluated as a pair rather than as individual oligos. In Step 6, the attempted replacement will be either for a pair of fP/B or for an individual rP oligo. For example, for a 20-plex BDA with 15 candidates for each fP/B pair and 30 candidates for each rP, there will be 15²⁰*30²⁰=1.2*10⁵³ possible sets of oligos.

### II. OPERATION OF MBDA IN QUANTITATIVE PCR (QPCR) ASSAYS

mBDA amplicons can be read out by qPCR using either an intercalating dye (e.g., SybrGreen, EvaGreen, Syto) or with amplicon-specific Taqman probes. The design of Taqman probes for qPCR is known to one of ordinary skill in the art of molecular probe design. The observed solution fluorescence thus can correspond to either the total quantity of amplified DNA, or the quantity of the specific amplicon that the Taqman probe targets.

To accommodate the increased number of oligonucleotide species (fP, B, and rP) in mBDA systems, primer and blocker concentrations are reduced from standard single-plex PCR and BDA reactions. There are two reasons for using lower oligo concentrations: (1) reduced oligo concentrations quadratically reduce the likelihood of primer dimer formation, and (2) high concentrations of ssDNA and dsDNA are known to inhibit PCR. In some embodiments, the total concentrations of all fP, B, and rP species in the final PCR solution are kept between 50 nM and 50 μM.

The most straightforward division of the total concentration among the different mBDA oligos is to have identical fP and rP concentrations, and B concentrations a fixed multiple of the fP concentration. For example, a 20-plex mBDA system targeting 2 μM total oligo concentration could have all fP and rP species each at 20 nM, and all B species each at 60 nM. The cycle threshold (Ct) value of the qPCR amplification can be used to detect or quantitate sequence variations in the enrichment loci of the mBDA. Depending on the exact nature of the readout (intercalating dye vs. Taqman probe), the Ct value reflects either a specific sequence variation, any variation at a locus or a group of adjacent loci, or any variation across all loci enriched by mBDA.

### III. OPERATION OF MBDA IN NEXT GENERATION SEQUENCING (NGS) ASSAYS

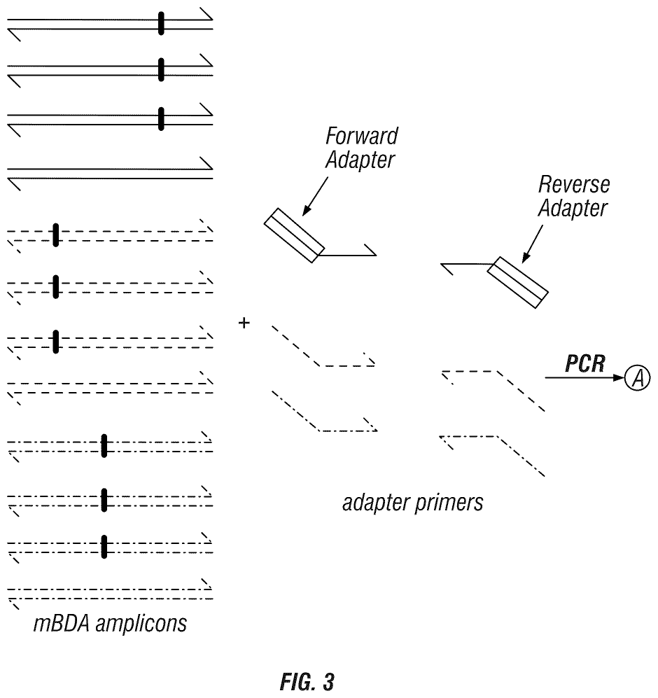

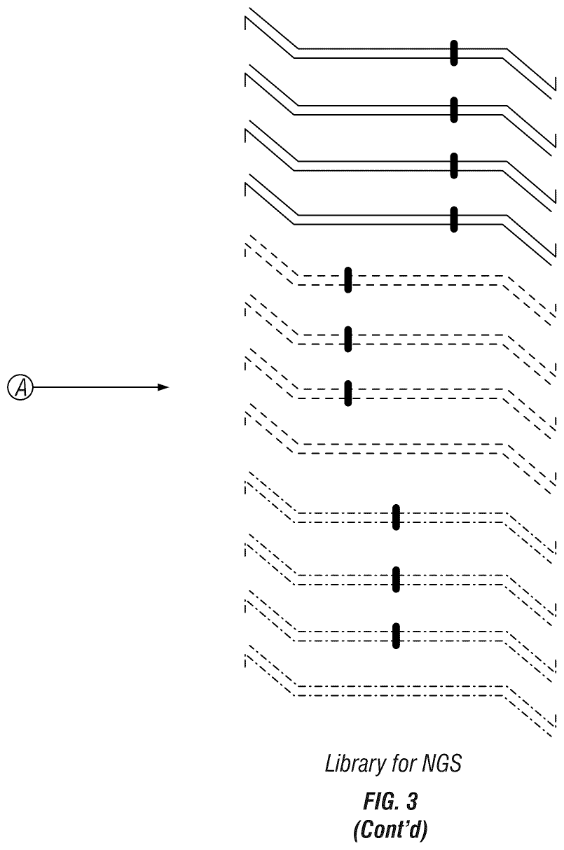

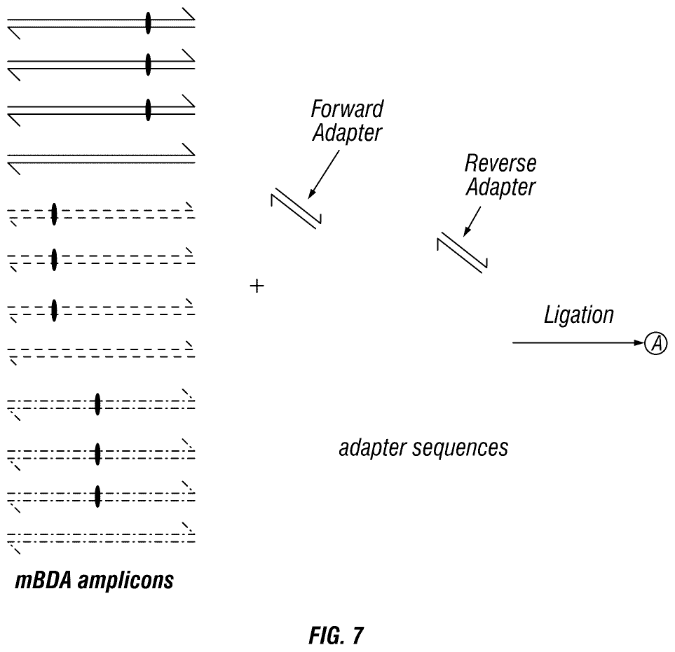

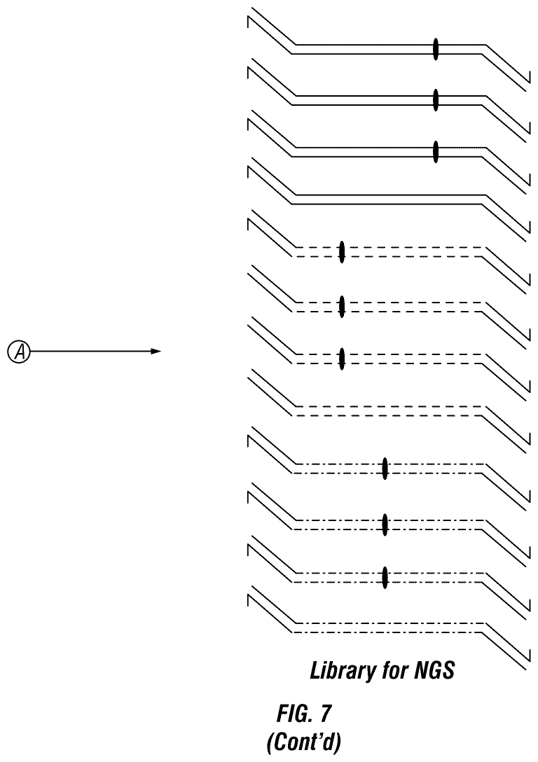

mBDA amplicons can be more precisely analyzed by downstream NGS. Commercial NGS systems (e.g., Illumina, Ion Torrent) require that special adapter sequences flank the sequence of interest (a.k.a. the insert). These can be appended to mBDA amplicons via either PCR or ligation, as shown in FIGS. 3 and 7.

For appending mBDA amplicons by PCR (FIG. 3), adapter primers bearing both universal NGS adapter sequences and gene-specific sequences are introduced to the mBDA amplicon mixture, and 2 or more cycles of PCR results in an amplicon mixture with sequencing adapters appended on either side of each mBDA amplicon. In some embodiments, the mBDA amplicon mixture is purified via size selection to remove primer dimers before appending adapter sequences. In some embodiments, the adapter-appended amplicons are subject to another round of PCR to append sample indexes or other adapter sequences. In some embodiments, the adapter-appended amplicons are subject to further purification or quality control before next-generation sequencing (NGS).

For appending mBDA amplicons by ligation (FIG. 7), the 5′ phosphate needed for ligation can be introduced to the mBDA amplicons through use of primers with 5′ phosphate modifications, or via kinase after mBDA. Unlike in the PCR method for introducing adapters, only 1 pair of universal adapter sequences is needed. However, this ligation approach requires a purification step to remove excess adapters and unligated amplicons.

NGS analysis of mBDA amplicons offers two significant advantages over NGS analysis of a library constructed from direct multiplex PCR amplification of a sample and that of libraries constructed from ligation and hybrid-capture. First, mBDA enriches rare mutations by 100- to 10,000-fold, allowing sensitive detection of rare sequence variants. All NGS platforms suffer from an intrinsic sequencing error rate that varies between roughly 0.1% (Illumina) and 8% (Oxford Nanopore). Without complex unique molecular identifier barcodes or other technologies to suppress sequencing error rates, the NGS intrinsic error rate limits the analytic sensitivity of NGS to rare mutations. For example, if the NGS error rate is 1%, then out of 1,000 reads to a locus, if 987 are mapped to A, 7 are mapped to T, 2 are mapped to C, and 4 are mapped to G, it would not be clear if a T variant actually exists, or whether the 7 reads mapped to T are simply due to sequencing error. When BDA enriches a T variant from 0.2% VAF to 40% VAF, then the T allele reads can be clearly distinguished against the sequence error background.

Unique molecular identifier (UMI) barcodes have been recently demonstrated to suppress NGS sequencing error, and are used by commercial products such as Roche Avenio and Guardant 360. However, UMIs increase the sequencing reads required (and thereby NGS cost) by roughly 10-fold, and furthermore are difficult to both experimentally implement and bioinformatically interpret.

The second advantage of sequencing mBDA amplicons is that the number of NGS reads required is significantly reduced compared to both standard NGS and NGS with UMIs. For example, a sample that has a particular mutation at 1% VAF may require 500 reads for standard NGS (5 reads out of 500), and 5000 reads for NGS with UMIs, but after mBDA enrichment to >80% VAF, just 10 reads is likely sufficient for a confident call of a positive detection of the said mutation.

NGS analysis of mBDA amplicons can also be used to inform adjustments of primer concentrations to achieve more uniform amplification of all mBDA amplicons. Different primer sequences bind to their respective targets with kinetics that can vary by more three orders of magnitude, and currently even the best biophysical models and machine learning algorithms are unable to predict primer binding rate constants with better than about 90% accuracy. Consequently, an initial design of mBDA oligos using the same concentrations for all fP and rP species will likely result in significant bias of reads, with some amplicons being sequenced to perhaps 10-fold higher depth than others.

The reads mapped to each mBDA amplicon in an initial NGS run can be used to inform the adjustment of fP and rP concentrations. Because the concentration ratio of fP and B should be maintained as a constant to achieve reproducible fold-enrichment of sequence variants, it is preferable to adjust rP concentrations based on observed reads. Experimentally, using [rP]_{new}=[rP]_{old}*(Reads_median/Reads_amplicon)\hat{ }X works well, where [rP]_{old} is the previous concentration of the reverse primer, Reads_median is the median reads mapped to each amplicon, Reads_amplicon is the reads mapped to the amplicon corresponding to said reverse primer, and X is a constant adjustment factor. For different NGS library preparation protocols, the optimal value of X may be 0.33, 0.5, or some other value between 0.25 and 1.

### IV. INTERPRETATION OF NGS RESULTS THAT UTILIZE MBDA ENRICHMENT

One embodiment of an algorithm to analyze NGS reads from FASTQ files is described below. Similar algorithms from FASTQ or SAM files can similarly be constructed by one of ordinary skill in the art of bioinformatic processing of NGS reads.
1. Trim off adapter sequences or parts of adapter sequences, if any, from all reads.
2. Remove reads with low quality score (e.g., median Q across <30).
3. Align trimmed sequences to reference amplicon sequence list, obtaining a sam/bam file. Alignment may be either end-to-end alignment to amplicon sequences with adapter sequences on both sides, or local alignment to the original mBDA amplicon sequences.
4. Clip the read sequence to the enrichment region of the relevant mBDA blocker, using the pileup column or through nearby conserved sequences.
5. Check the quality of the read at the enrichment loci, discarding the read if the Q score is <30 at the loci of interest. Alternatively, if paired end sequencing is allowed, discarding the paired reads if they disagree with each other at the loci of interest.
6. Tabulate the number of reads mapped to the wildtype amplicon sequence, and any expected variant sequences, and other variant sequences.

From the above algorithm, a reads fraction (RF) is calculated for each mBDA amplicon. Given RF and an estimate on the fold-enrichment (E) achieved by mBDA for that locus/variant, the VAF of the variant in the initial sample can be estimated via VAF=RF/(E *(1−RF)+RF). Because of the NGS intrinsic error issues discusses previously, the user may wish to discard consideration of variants with RF below a threshold (e.g., 1%).

### V. APPLICATION CONSIDERATIONS FOR NONINVASIVE CANCER PROFILING VIA CELL-FREE DNA IN PLASMA

Cell-free DNA (cfDNA) in plasma are derived from dying cells (due to apoptosis, necrosis, or immune system attack). Because cfDNA is cleared from the bloodstream via the kidneys with a reported half-life of between 10 and 60 minutes, cfDNA offers a snapshot in time of the body. Numerous studies have shown that tumor-derived cfDNA detected in patients consistently provides similar mutation profiles as biopsy samples. Because cfDNA requires only a minimally invasive blood draw, rather than invasive biopsies that risks infection, cfDNA analyses have also been referred to as liquid biopsies.

Unlike DNA derived from tumor samples, however, cfDNA is much shorter, with an average length of between 160 and 180 nt. Because the breakpoints for cfDNA are more or less uniformly distributed, use of longer amplicons risks losing sensitivity. For example, if the average length of cfDNA is 160 nt, and the length of a mBDA amplicon is 120 nt, then only about 1 in 4 cfDNA molecules bearing an enrichment locus of interest will have a breakpoint outside the amplicon and be amplifiable. For this reason, mBDA fP and rP sequences should be designed such that all amplicons are shorter than 120 nt, and preferably shorter than 90 nt.

On the other hand, the short length of cfDNA means that the risk of unintended longer amplification products, e.g., from rP species from different BDA systems, is essentially nonexistent. This means that for mBDA oligo sets designed for cfDNA analysis, the design constraint of having all BDA systems close to each other target the same (+) or (−) template strand does not apply, allowing more options for B sequence selection.

### VI. APPLICATION CONSIDERATIONS FOR CELL LINE CONTAMINATION ASSAYS

Human cell line samples are used both by academic and industry research laboratories, and by cell therapy providers (e.g., stem cell therapy or autologous CAR-T therapy). Potential cell contamination is a significant concern for these industries, because it could lead to incorrect scientific conclusions for research and immune reactions leading to adverse patient outcomes for therapeutics.

The cell contamination detection problem can be classified into two primary cases: (1) problems where the base (desired cell) genotype is known, but the contaminant is unknown, or (2) problems where the contaminant genotype is known, but the base genotype is unknown. In both classes of problems, mBDA can be used to enrich for nonpathogenic single nucleotide polymorphisms (SNPs).

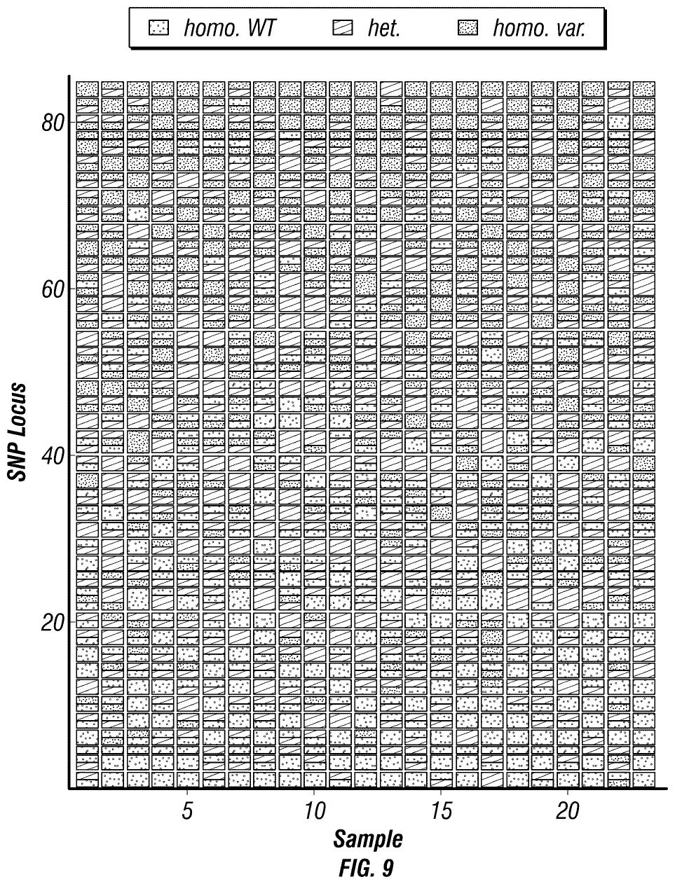

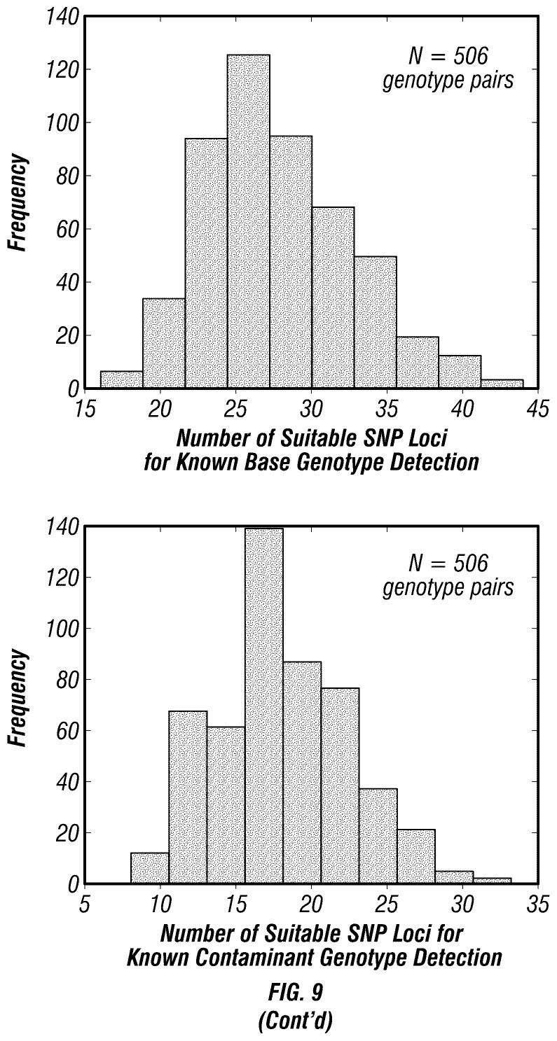

Traditionally, criminal forensics and other fields use short tandem repeat (STR) profiling for determining genomic identity, but STR profiling requires electrophoresis and is not convenient to do in a high throughput manner, and furthermore has limited sensitivity. A panel of roughly 80 SNPs, each selected so that a variant allele has a population frequency between 5% and 95%, is with high probability capable of distinguish any two non-identical genomes. FIG. 9 shows the SNP genotype profiles of 23 individuals (volunteers from the Houston area). For any pair of individuals, there are on average more than 25 SNP allele differences out of the 85 characterized, and always more than 15 for all pairs analyzed. Mathematics suggests that any two people alive in the world today would still likely be different in at least 6 of the 85 SNPs profiled. Thus, nonpathogenic SNPs can serve as reliable markers of genomic identity.

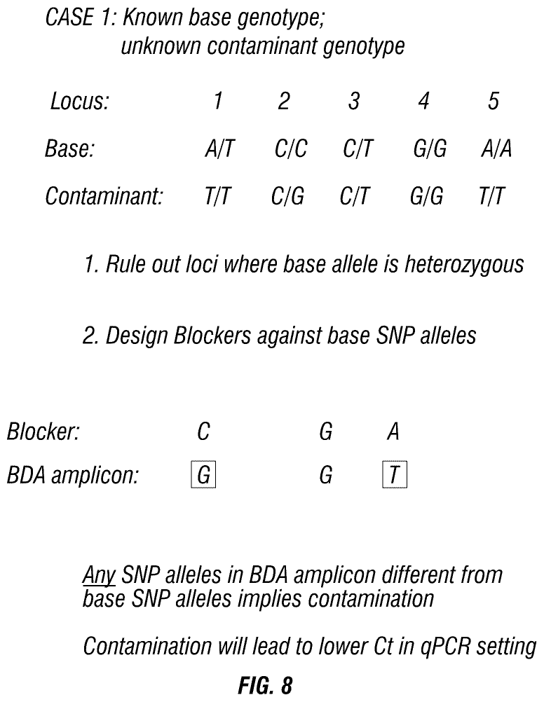

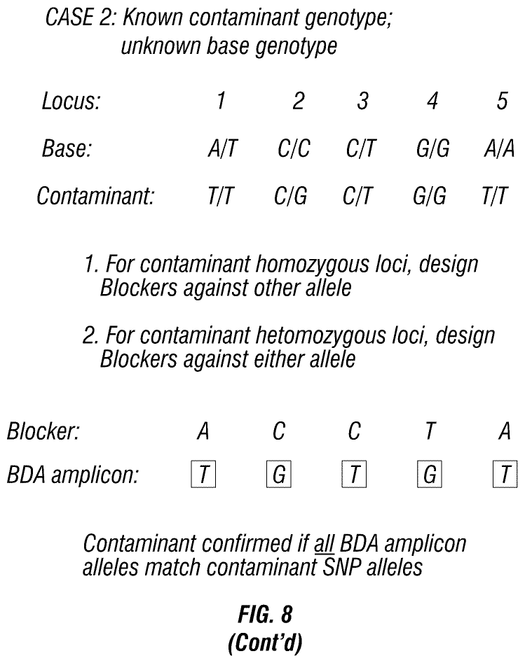

For the first class of problems (known base genotype, Case 1), which is more relevant to cell therapy facilities, mBDA oligos can be designed to selectively block the amplification of all homozygous SNP alleles specific to the base genotype (FIG. 8). In other words, mBDA blockers are designed to suppress the homozygous SNP alleles of the base cell line. The amplification and detection of any alternative allele in any of the mBDA systems would suggest a contamination. Importantly, this method does not require prior knowledge of contaminant genotype and should be generally applicable to all contaminants that are not genetically identical to the base. Assuming that the contaminant cell line is not identical to the base cell line in the panel of SNPs profiled, there will be at least 1 SNP locus that is not suppressed by the blocker and will amplify very efficiently. This results in both an alternative allele in NGS data, and a shift to a lower Ct value in qPCR; both can be used for cell line contamination detection. There is a small probability that the contaminant has the same SNP alleles within the panel observed, but this probability exponentially decreases with the number of SNP sites characterized, and is expected to be very small for a panel of 80 SNPs.

The second class of problems (known contaminant genotype, Case 1) is more relevant to academic research labs, where HeLa cells are known to be a primary contaminant. Although it is of course possible to perform genomic profiling of the base cell line, doing so may be inconvenient or too costly for cell lines that see only limited use as part of a larger set of experiments. Cellular contamination can be detected based on only the SNP allele profile of the contaminant, without knowledge of the base SNP genotype (FIG. 8). mBDA oligos can be designed to suppress SNP alleles other than the ones present in the contaminant genome. Thus, presence of the contaminant would mean that all mBDA amplicons will show contaminant-specific SNPs. At loci where the contaminant is heterozygous, either allele may be blocked. Assuming that the base cell line is not identical to the contaminant in the panel of the SNPs profiled, a lack of contamination means that some of the blockers will suppress amplification. In contrast, if there is contamination, all alternative alleles will be efficiently amplified, and the Ct value of a qPCR reaction will be low.

There is also a third case of cell contamination detection, in which the SNP genotypes of both the base and the contaminant are known. This is a relatively easier problem that can be solved by single-plex BDA (see U.S. Patent Appln. Publn. No. 2017/0067090, which is incorporated herein by reference in its entirety for all purposes).

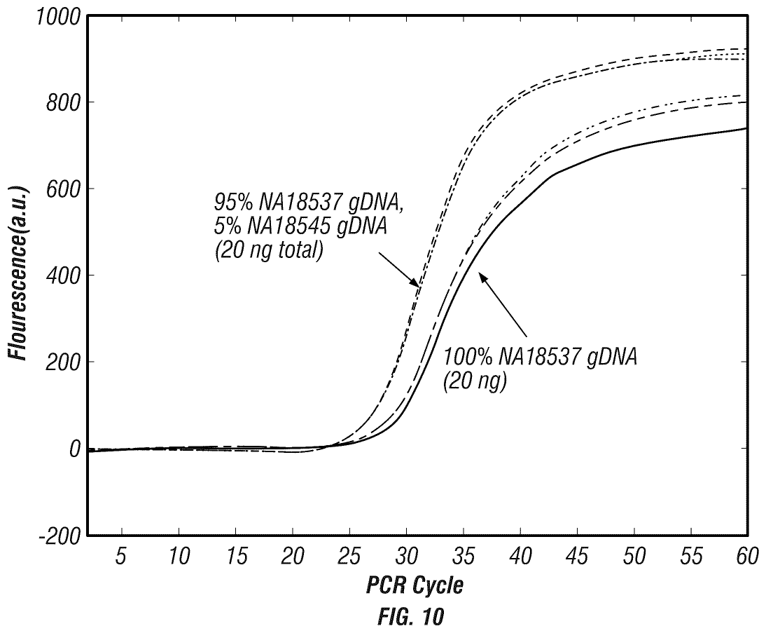

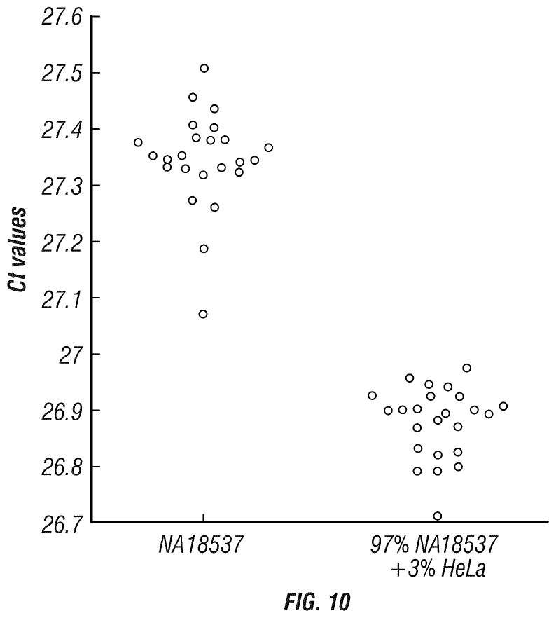

In some embodiments of the cellular contamination detection application of mBDA, qPCR is used as the readout. FIG. 10 shows results in which the NA18537 human cell line gDNA serves as the base. An 80-plex BDA system was designed to block homozygous SNP alleles of NA18537. Two different contaminants, NA18562 and HeLa, were tested at 5% and 3%, respectively. In both cases, qPCR showed an observable decrease in Ct for the contaminated samples vs. the pure NA18537 samples. In other embodiments of the cellular contamination detection application of mBDA, NGS can be used as the readout. Doing so should also provide reliable detection of cellular contamination at a less than 0.1% VAF limit of detection.

### VII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—NGS Experimental Results

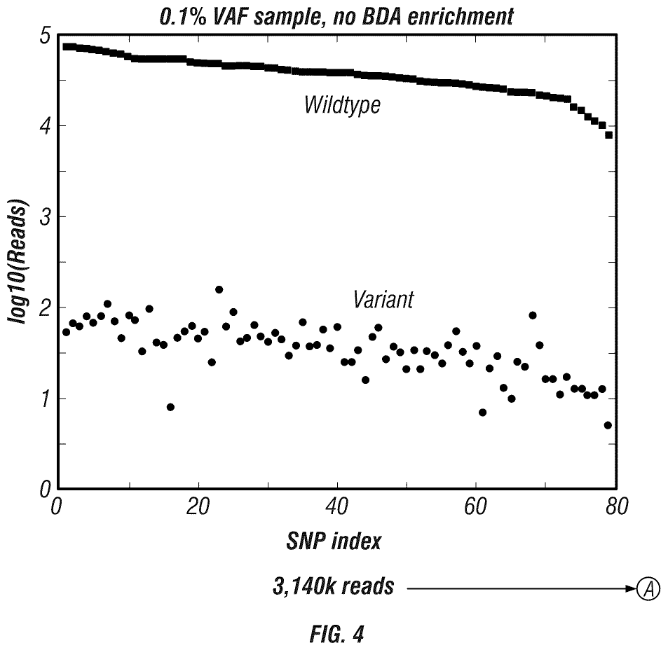

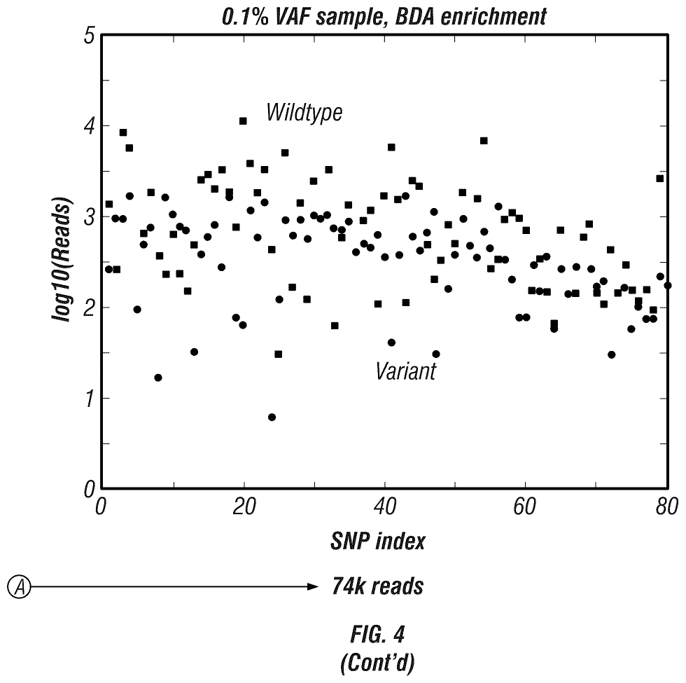

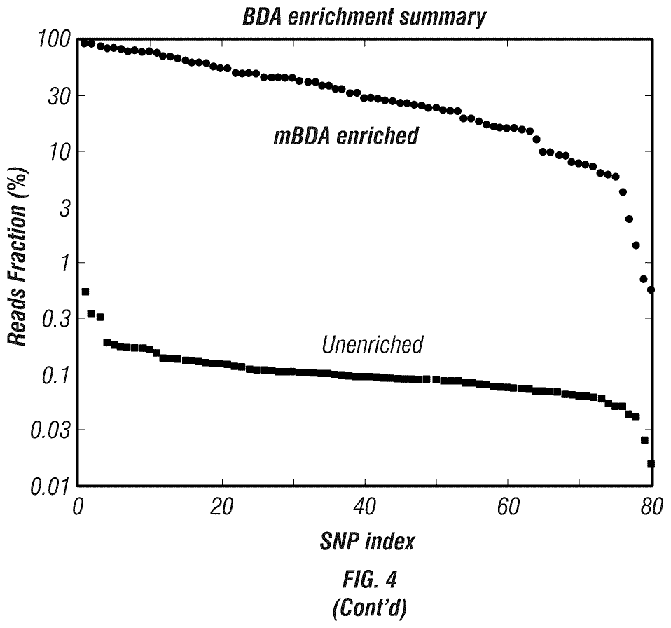

A cell line genomic DNA sample was constructed by mixing 99.9% NA18537 and 0.1% NA18562 (50 ng total). The left panel of FIG. 4 shows the number of reads mapped to the wildtype (NA18537) and variant (NA18562) alleles for each of 80 loci, following a standard 80-plex amplicon sequencing. The number of reads mapped to the variant allele is roughly 1000-fold lower than for the wildtype allele for all loci, consistent with expectations. The middle panel of FIG. 4 shows the number of reads mapped to the wildtype and variant alleles for each of the 80 loci following 80-plex mBDA. The number of reads mapped to the variant alleles is now comparable to the number of reads mapped to the wildtype alleles. Importantly, all variant alleles were sequenced at comparable or higher depth than the unenriched library, but the total number of NGS reads used is 40-fold lower. This represents a potential 40-fold savings in NGS analysis of complex DNA samples. The right panel of FIG. 4 shows the fraction of reads mapped to the variant allele with and without mBDA enrichment.

### Example 2—Inferring Initial VAF from Post-mBDA NGS Reads

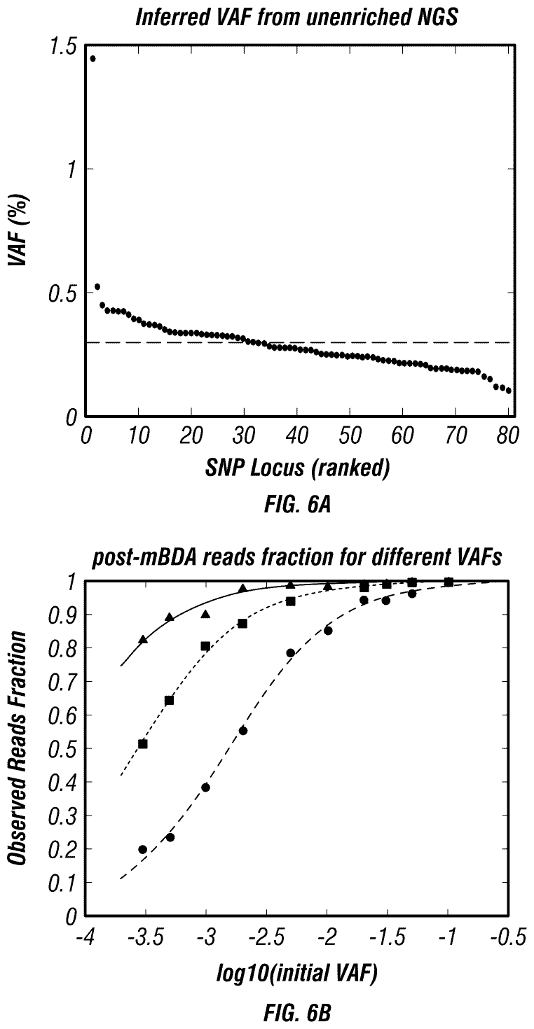

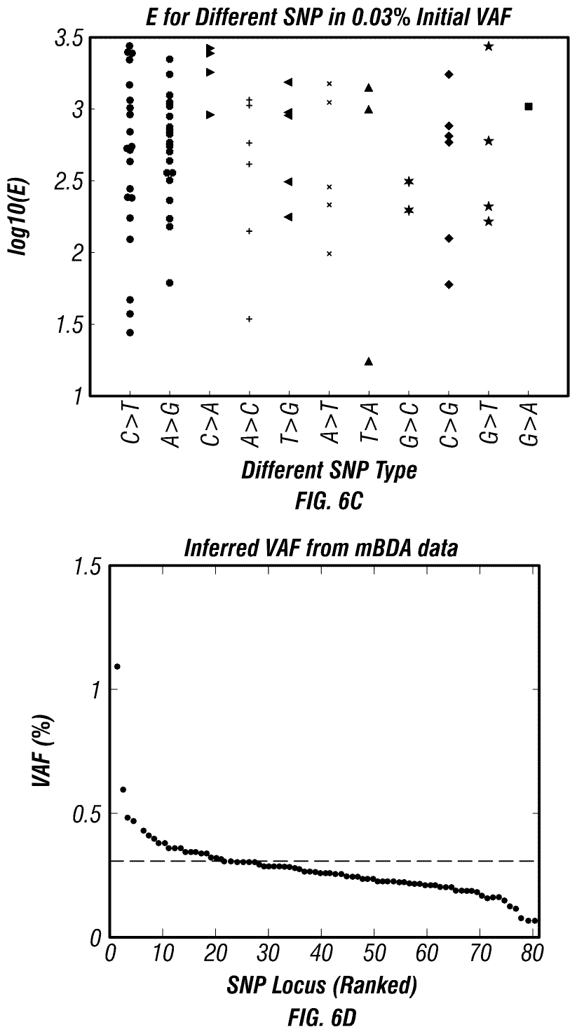

FIG. 6A shows the inference of VAF from unenriched multiplex PCR library, based on fraction of reads mapped to variant allele at each locus. The dashed horizontal line shows the expected 0.1% VAF; inferred VAF varies slightly from the expected due to NGS sequencing bias. FIG. 6B shows the relationship between initial VAF and post-mBDA NGS reads fraction mapped to variant allele. Different variants at different loci have different fold-enrichment E, but the relationship between the reads fraction and the VAF are as expected (sigmoidal line shows expectation based on theory and best-fit fold enrichment E). Here, all input samples were 50 ng of genomic DNA, and subject to 23 cycles of mBDA. FIG. 6C provides a summary of fold enrichment E for different SNP types. There does not appear to be significant sequence bias on E. FIG. 6D shows the inferred VAF from reads fraction (from post-mBDA NGS) and fitted E values. The inferred VAF values show a similar distribution as those based on direct analysis of multiplex PCR NGS.

### Example 3—Using mBDA to Detect Variant DNA Sequences with Low Variant Allele Frequency (VAF) Using Low-Depth Next-Generation Sequencing (NGS)

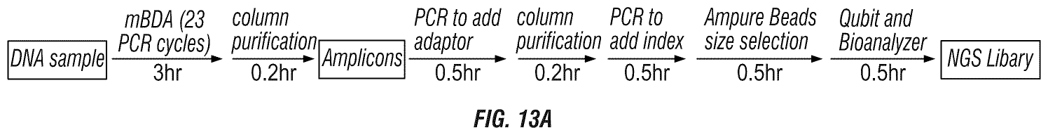

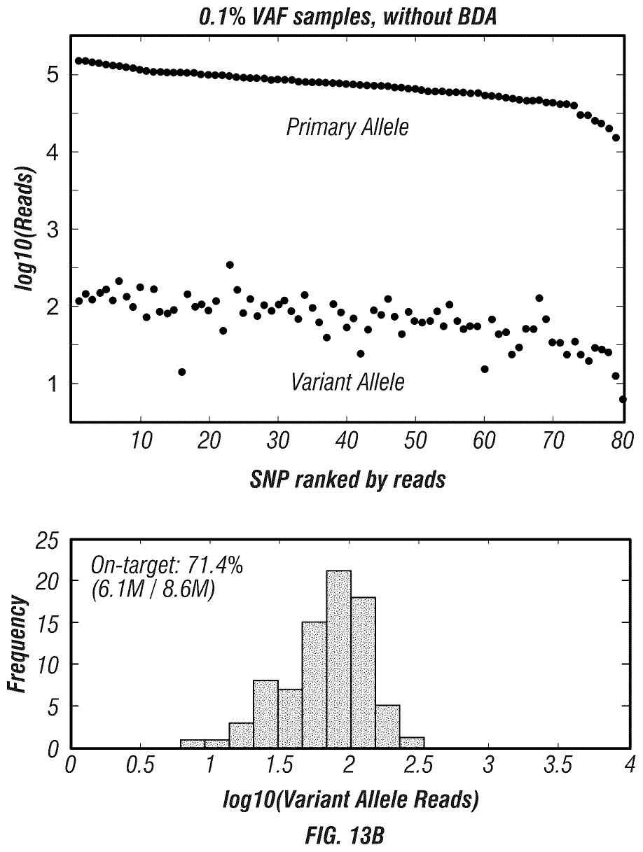

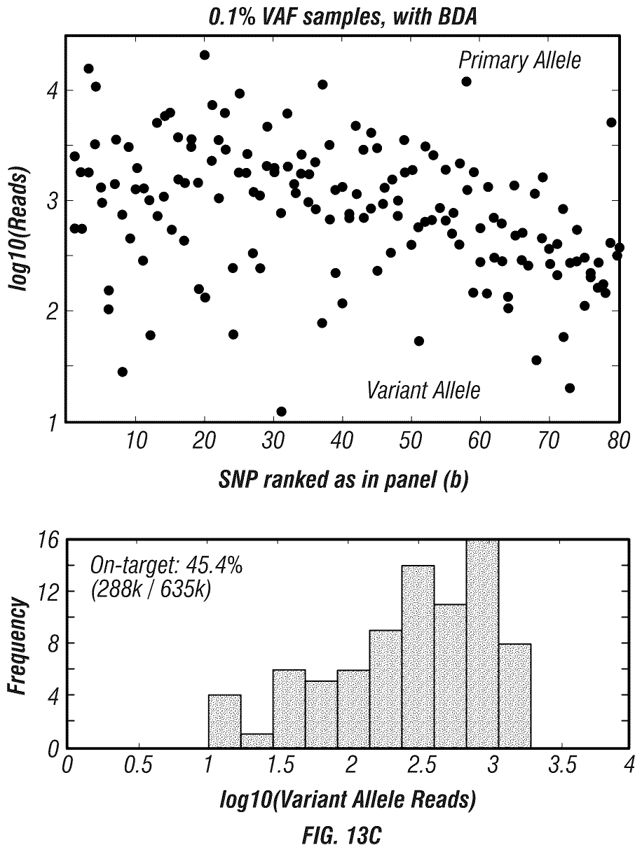

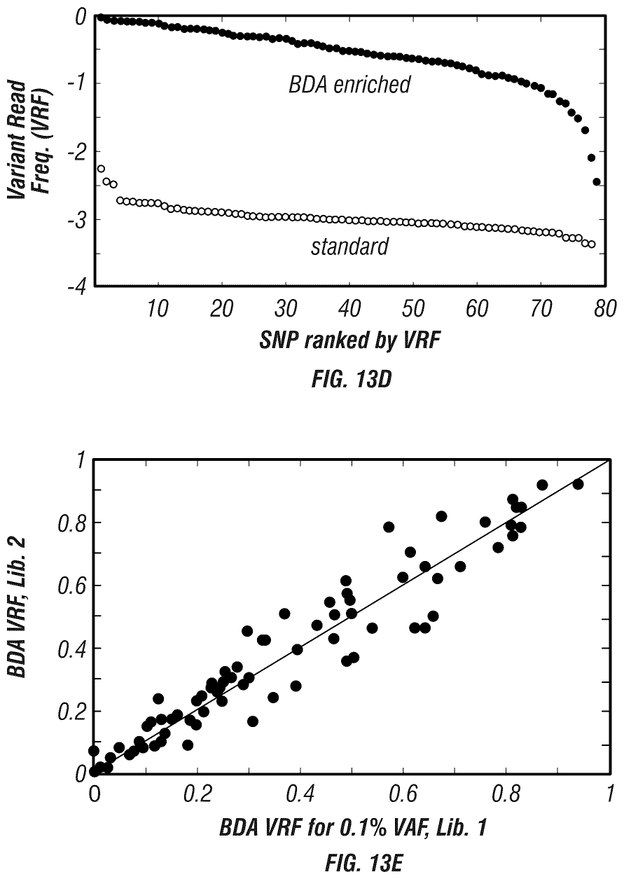

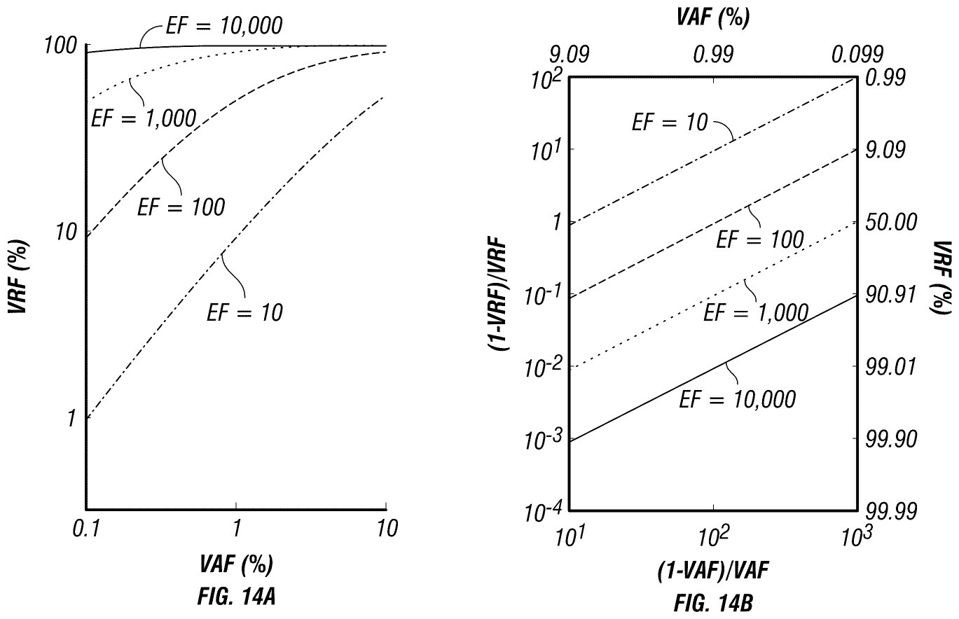

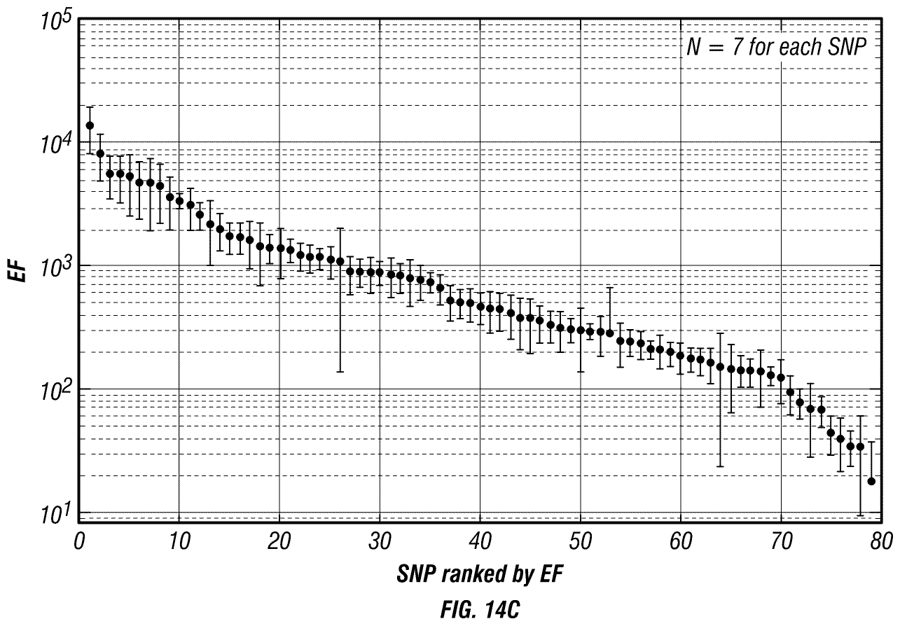

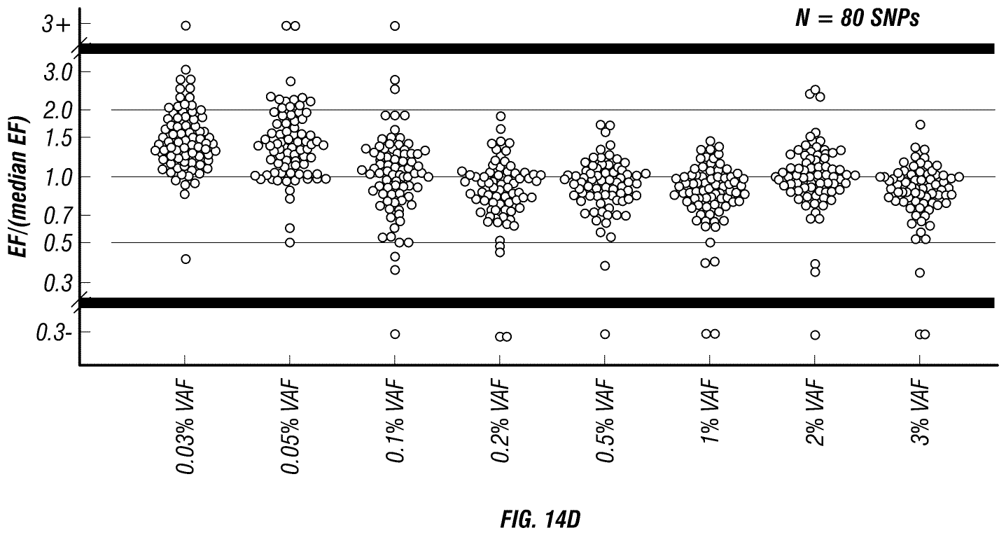

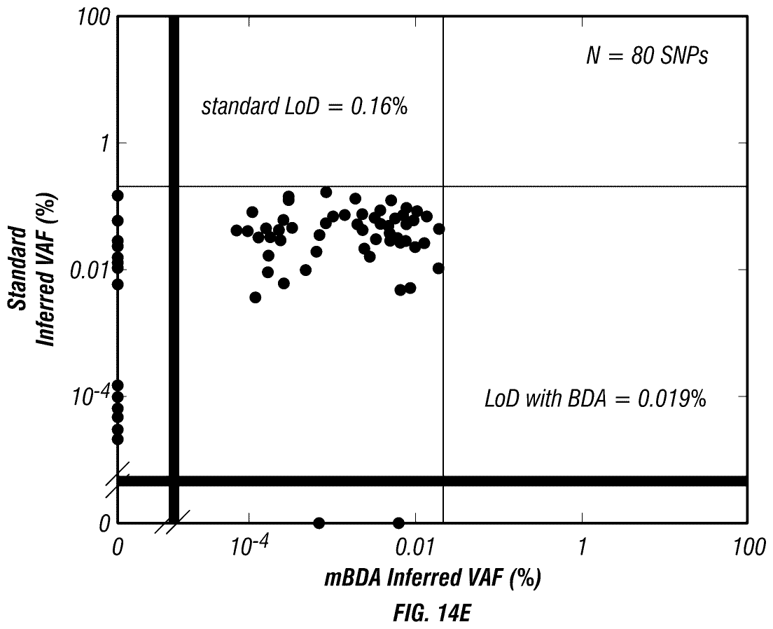

A summary of the mBDA NGS library preparation workflow is shown in FIG. 13A. And a summary of NGS results on an amplicon library without using mBDA are shown in FIG. 13B. Here, the sample was 50 ng of a 99.9%:0.1% mixture of NA18537 and NA18562. The 80 SNPs were selected such that NA18537 and NA18562 are homozygous for different alleles. 8.6 million NGS reads were used for this library, in order to ensure at least 5× sequencing depth on the variant alleles. Note that such standard amplicon sequencing is unable to confidently call variants at 0.1% VAF, due to the existence of NGS intrinsic error (see FIG. 14E). A summary of NGS results for the mBDA NGS library on the sample 0.1% VAF sample are provided in FIG. 13C. Compared to the library in panel (FIG. 13B), 13-fold fewer NGS reads were used, but the all variant alleles were sequenced to at least 10× depth. Thus, mBDA reduces the NGS required for rare variant profiling by at least a factor of 25. A summary of the variant read fraction (VRF) for each SNP locus in the libraries described in panels (FIG. 13B) and (FIG. 13C) are provided in FIG. 13D. The standard amplicon NGS library shows roughly 0.1% median VRF, as expected. The mBDA NGS library exhibits roughly 30% median VRF, indicating that variant SNP alleles are enriched typically by 300-fold. The reproducibility of the VRFs in two replicate mBDA NGS libraries using the 0.1% VAF sample is shown in FIG. 13E.

### Example 4—Quantitating Variant VAFs Based on mBDA NGS VRF

The theoretical relationship between VRF and VAF for different allele enrichment-fold (EF) is shown in FIG. 14A. EF values are expected to vary for different SNPs, but be conserved across different experiments and VAFs for the same SNP allele. The relationship between (1-VRF)/VRF and (1-VAF)/VAF is expected to be linear, with a slope of with 1 and an intercept of log 10(EF) (FIG. 14B). This relationship allows simple and unbiased inferences of EF based on calibration mBDA NGS libraries using known VAF inputs. A summary of inferred EFs for each of the 80 variant SNP alleles using NA18537 as the primary alleles are shown in FIG. 14C. Dots show mean values and error bars show 1 standard deviation, based on 7 calibration mBDA NGS libraries using 0.03%, 0.05%, 0.1%, 0.2%, 0.5%, 1%, 2%, and 3% VAF samples. Relative EF values for different VAF inputs are shown in FIG. 14D. The EF/(median EF) ratio can also be interpreted as the quantitation error for the calibration samples with known VAFs; e.g. a ratio of 2 indicates under-estimation of VAF by a factor of 2. For the calibration samples, a large majority of SNPs across all 7 VAFs are quantitated accurately to within a factor of 2. Ideally, EF values should be identically distributed for all VAFs; however, a slight upwards bias was observed for EFs at very low VAFs below 0.1%. VAF limit of detection (LoD) for standard amplicon NGS vs. mBDA NGS is shown in FIG. 14E. Here, the LoD is defined as the maximum inferred VAF from a purely wildtype (0% VAF) sample; the red dots show the inferred VAFs for each of the 80 SNPs. For mBDA NGS, the VAF is calculated based on the median EF from the 7 EF values summarized in panels (FIG. 14C) and (FIG. 14D). For standard amplicon NGS, the VAF is calculated as simply the VRF. BDA improves the VAF LoD by more than 8-fold vs. standard amplicon NGS.

### Example 5—Detection of Cell Line Contamination Using 80-Plex mBDA and qPCR Readout

These experiments assume known base SNP genotype (of NA18537). The sequences and concentrations of primers and blockers used are shown in Table 1. The qPCR temperature cycle consisted of 2 minutes at 95° C. followed by 5 minutes at 60° C. The left panel of FIG. 10 shows triplicate qPCR traces of a pure NA18537 sample vs. a NA18537 sample with 5% NA18545 contaminant. The right panel of FIG. 10 shows a summary of Ct values observed for 24 replicate experiments of pure NA18537 and of NA18537 contaminated with 3% HeLa gDNA. Importantly, neither set of experiments utilized any genomic/genotypic information regarding NA18545 or HeLa.

### Example 6—Detection of Minute Cell Line Contamination Using Multiplex Blocker Displacement Amplification (mBDA)

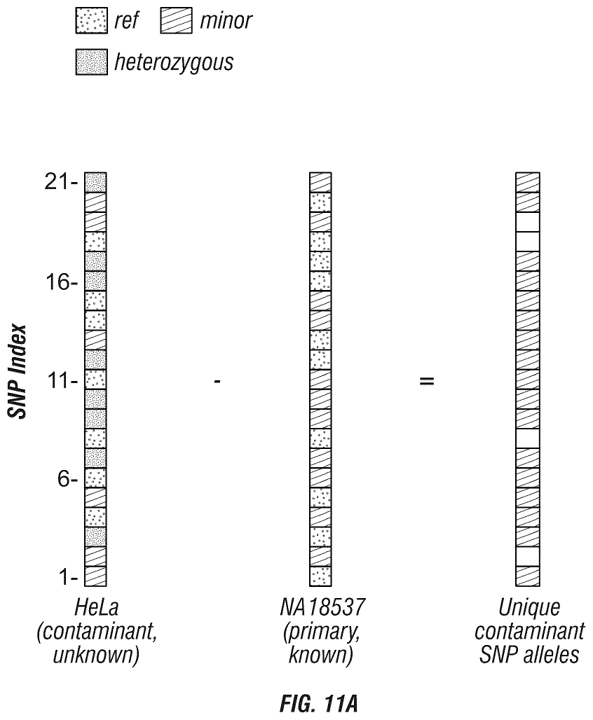

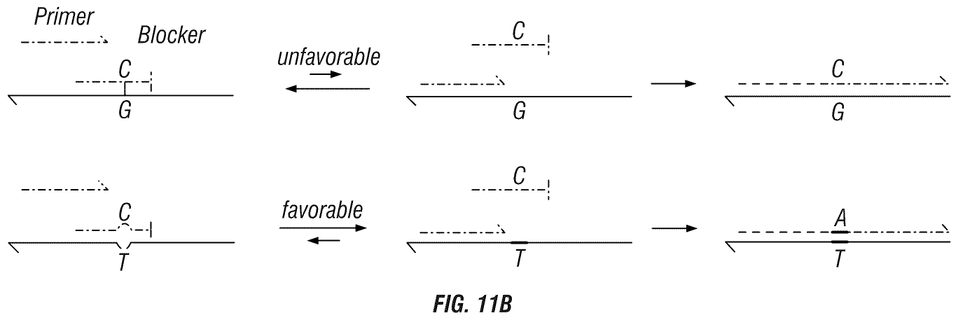

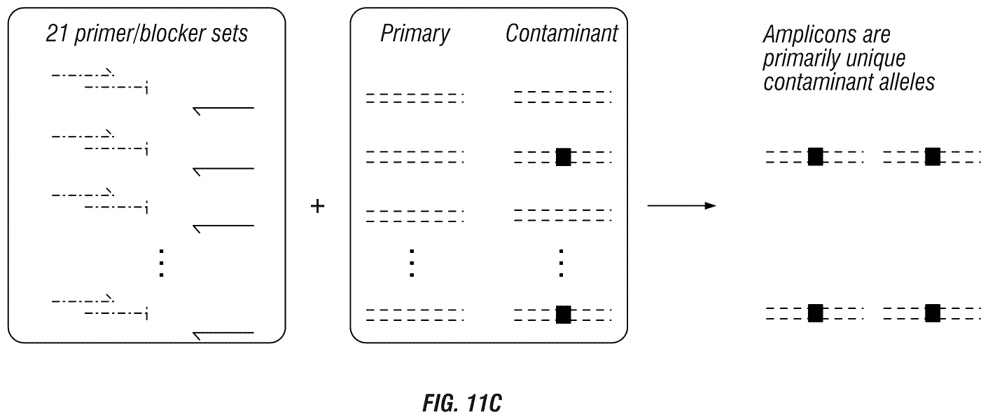

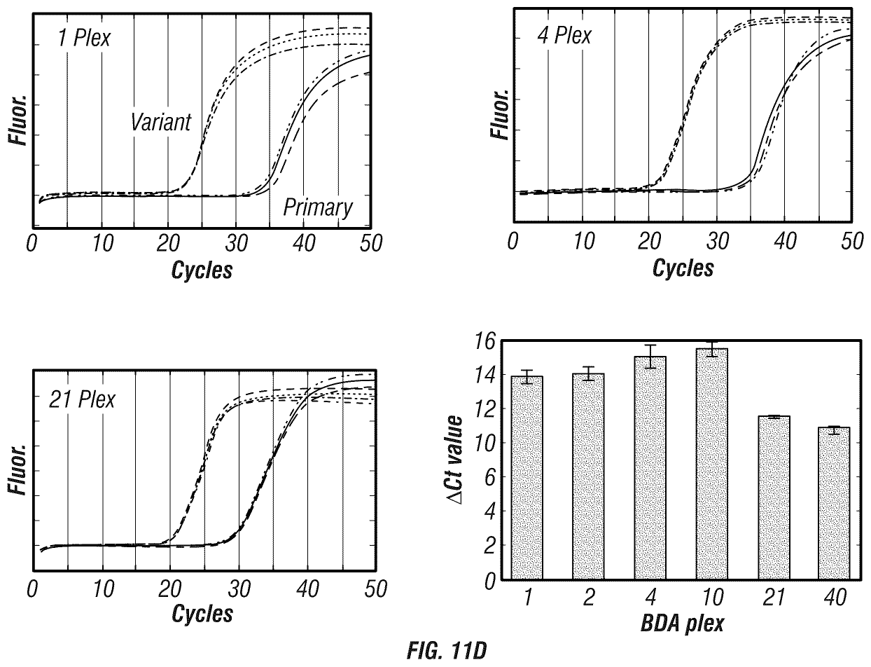

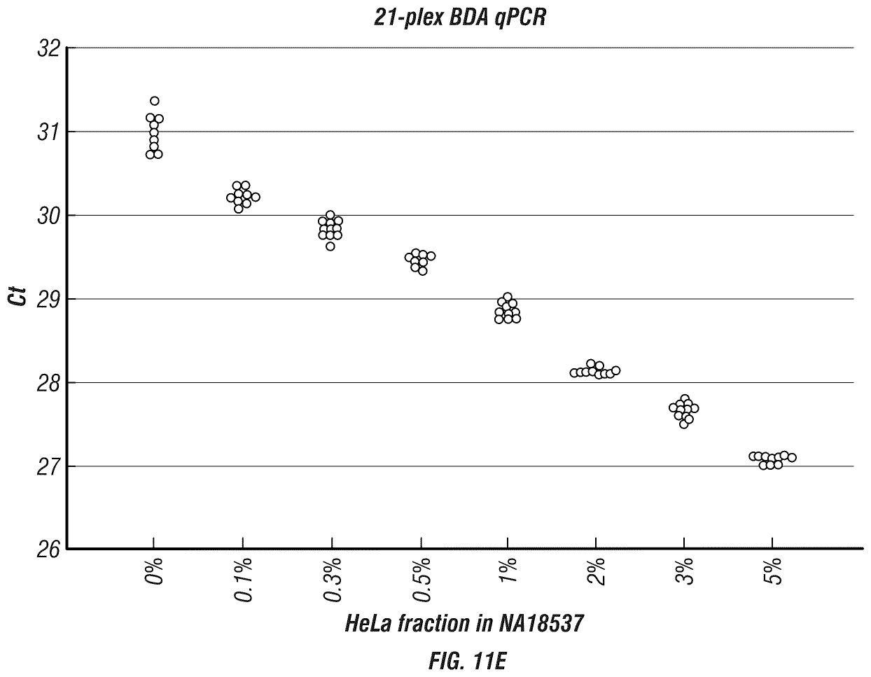

Given a known human cell line (here NA18537, referred to as the primary cell line), potential contamination by any other human cell line was sought to be detected. To this end, a list of SNPs in which the primary cell line is homozygous (either for the human reference allele, or the minor allele) was created. The unknown contaminant (here, HeLa) is overwhelming likely to differ in genotype in at least one SNP from the primary cell line, when the list of SNPs is sufficiently large. Detection of these unique contaminant SNP alleles allows detection and approximate quantitation of contamination without genotype information of the contaminant (FIG. 11A). BDA employs a rationally designed Blocker oligonucleotide that competes with the forward primer in binding to a DNA template. Here, the Blocker is designed to be perfectly complementary to the DNA template bearing the primary SNP allele, and mismatched to the variant SNP allele (FIG. 11B). This results in the variant SNP allele being preferentially amplified during PCR. A 21-plex mBDA set was designed against the primary alleles for all 21 SNP positions selected (FIG. 11C). Any contaminant present will have its unique contaminant SNP alleles be preferentially amplified, resulting in lower cycle threshold (Ct) values when using a double-stranded DNA intercalating dye such as SybrGreen I. mBDA maintains a large Ct difference between the primary and variant alleles in highly multiplexed reactions (FIG. 11D). Here, the variant DNA template tested is NA18562, which is homozygous variant in all selected SNPs. Different frequencies of HeLa contamination in NA18537 can be detected via mBDA using qPCR (FIG. 11E). The beeswarm plot shows the observed Ct values for 12 independent reactions for each sample. Even the 0.1% HeLa contaminant can be confidently distinguished from the pure NA18537 gDNA sample. Input quantity was 20 ng for all experiments summarized here.

### Example 7—Application of the mBDA Approach to Detecting Arbitrary Human Cell Line Contaminants

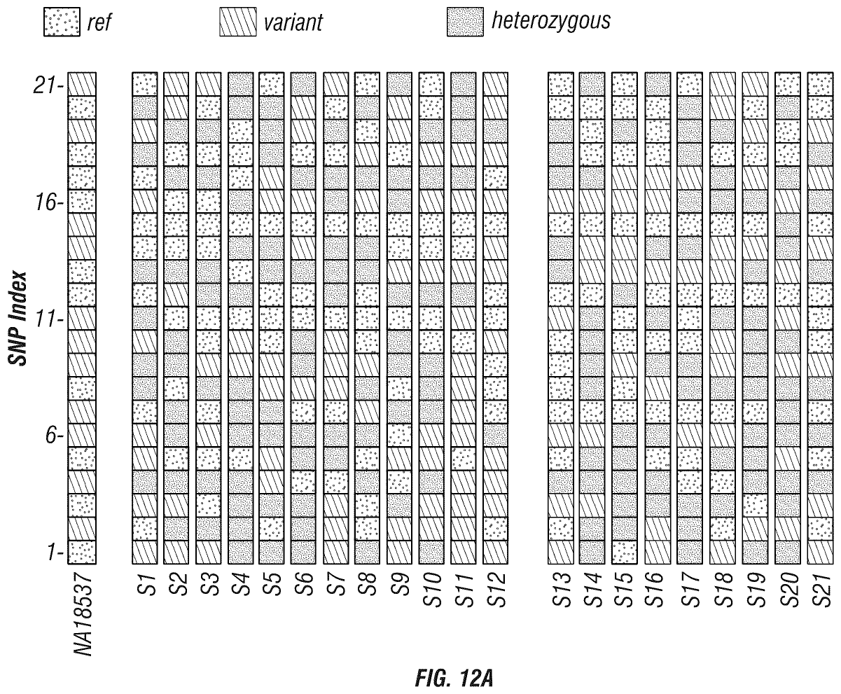

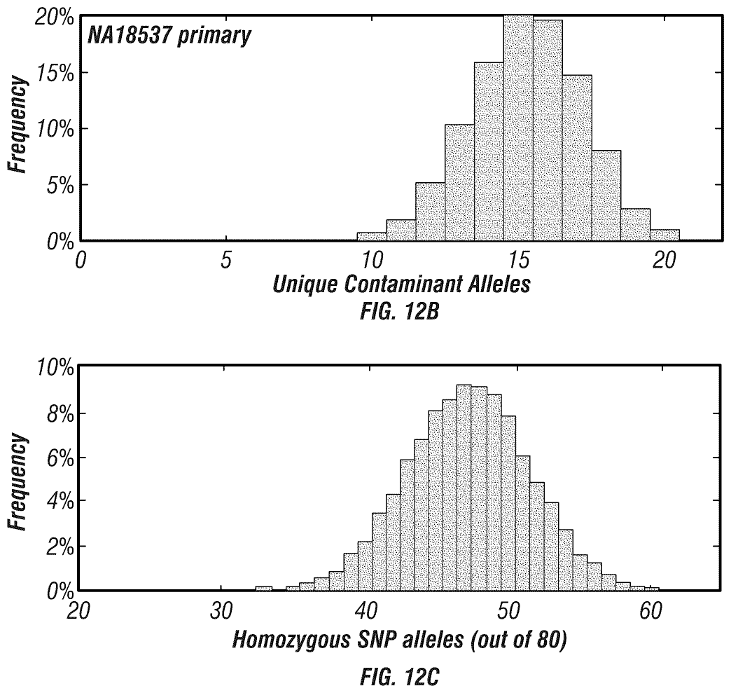

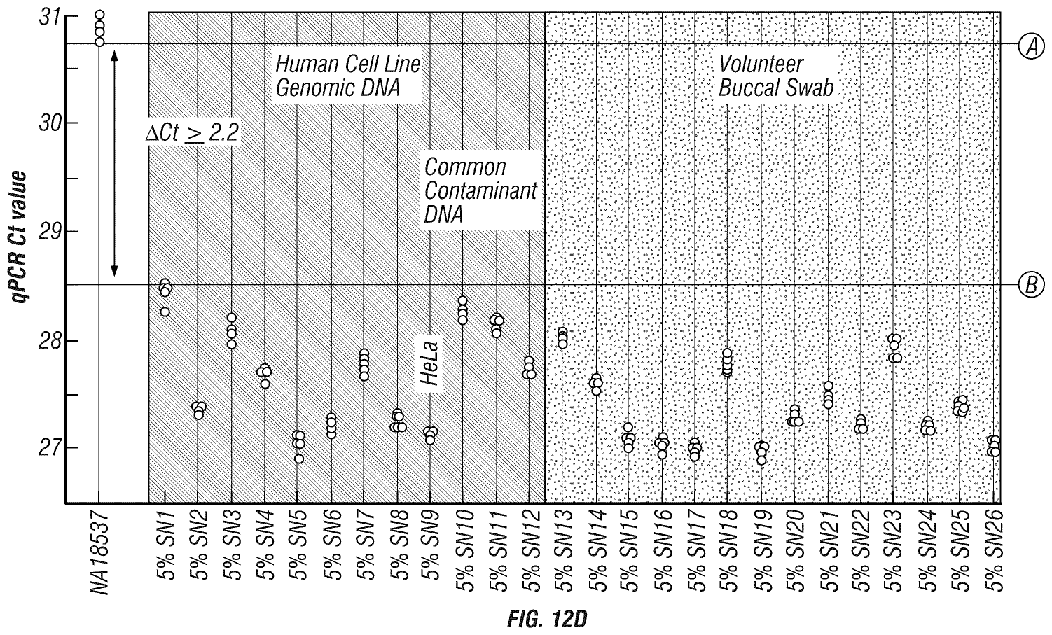

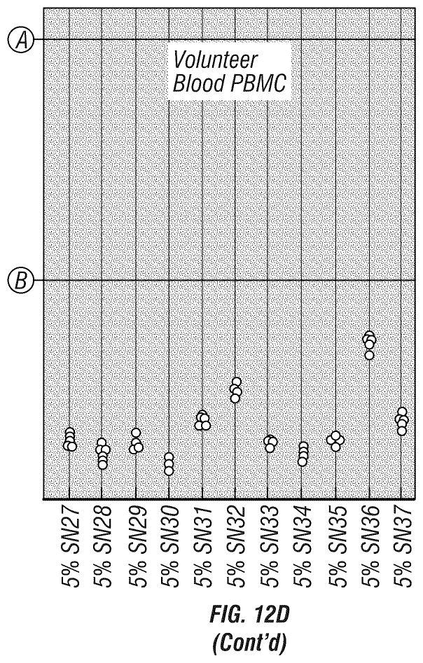

SNP genotypes for 21 different contaminant samples were analyzed (FIG. 12A). S1 through S12 are cell lines purchased commercially, while S13 through S21 are deidentified DNA samples from volunteers. Note that S9 corresponds to HeLa. Simulated distribution of the number of unique contaminant alleles out of the 21 SNPs observed, against NA18537, was based on reported SNP population allele frequencies and assuming independence between SNPs (FIG. 12B). In all 100,000 simulation cases, there were at least 9 unique contaminant alleles, indicating that detecting of an arbitrary contaminant is overwhelmingly likely. An 80-plex mBDA set was designed and used to simulate the distribution of the number of homozygous SNPs for an arbitrary cell line, based on reported SNP population allele frequencies and assuming independence between SNPs (FIG. 12C). In all 100,000 simulation cases, there were at least 31 SNPs out of the 80 that were homozygous, indicating that selection of a 21-plex subset of the 80-plex mBDA set for developing a qPCR kit to detect contamination in new cell line is overwhelmingly likely. FIG. 12D provides a summary of qPCR results for detection of 5% contaminant in NA18537 with 37 different human DNA contaminants. Because the different contaminants have different numbers and groups of unique contaminant alleles, there is some difference in the Ct difference between contaminated and pure NA18537 (ΔCt). Six replicate qPCR reactions were performed for each sample, using 20 ng input DNA.

### Example 8—Detection and Quantitation of DNA Contamination Using mBDA NGS

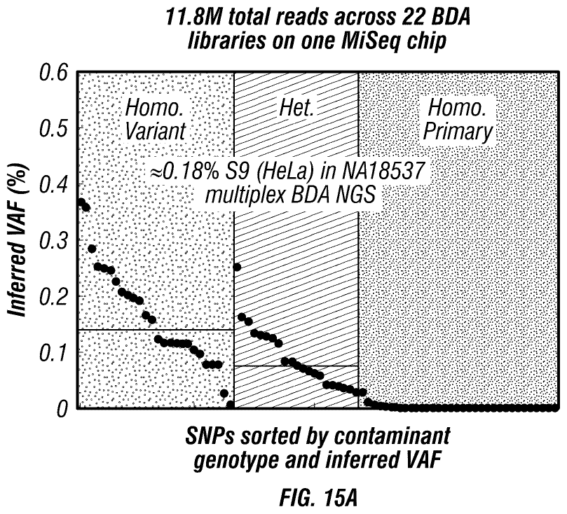

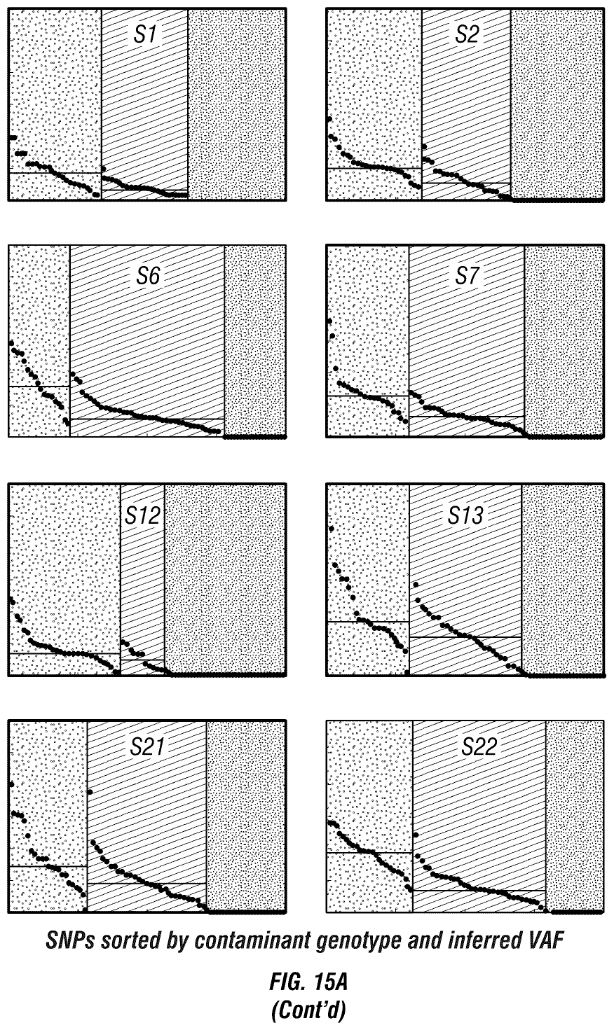

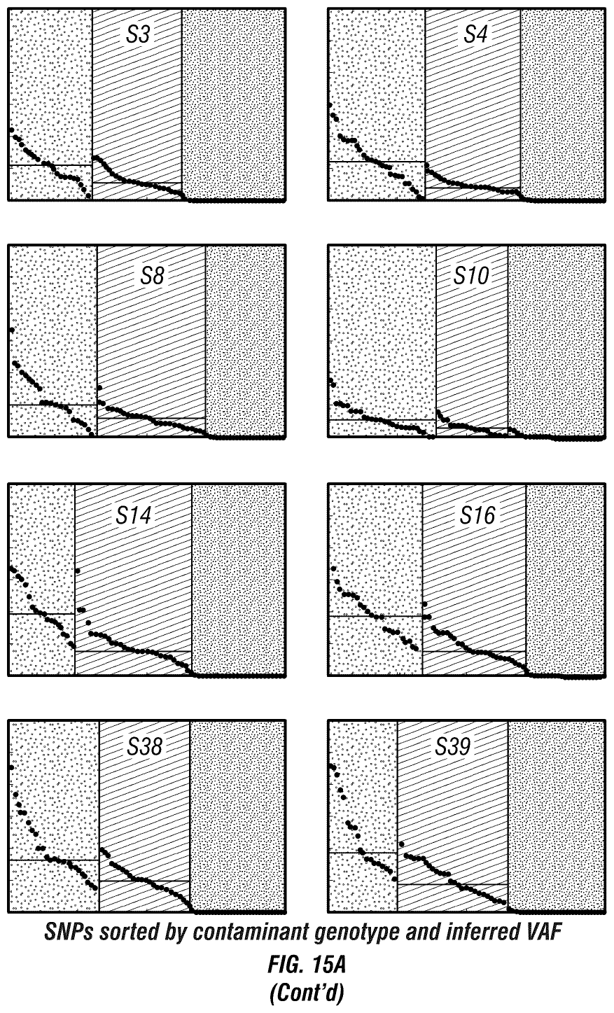

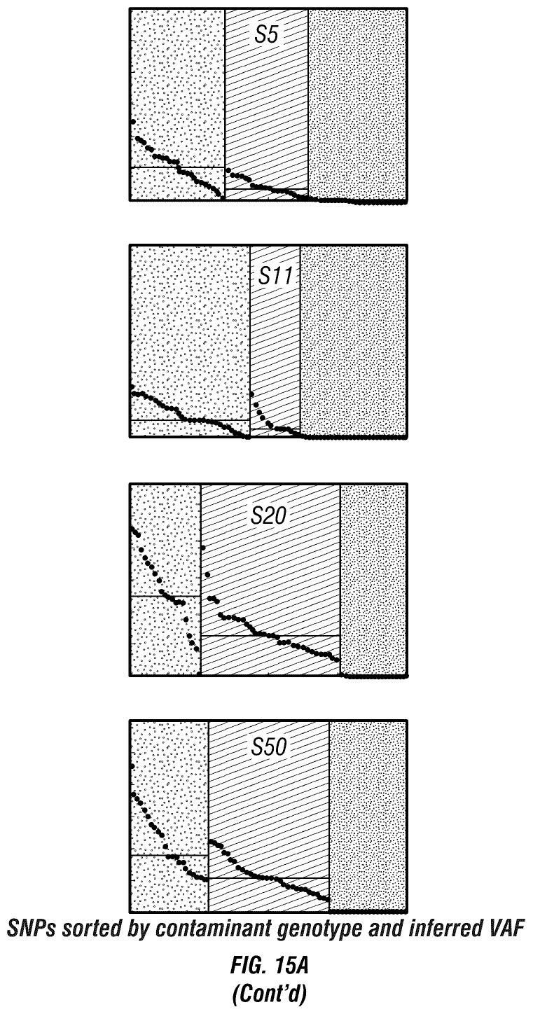

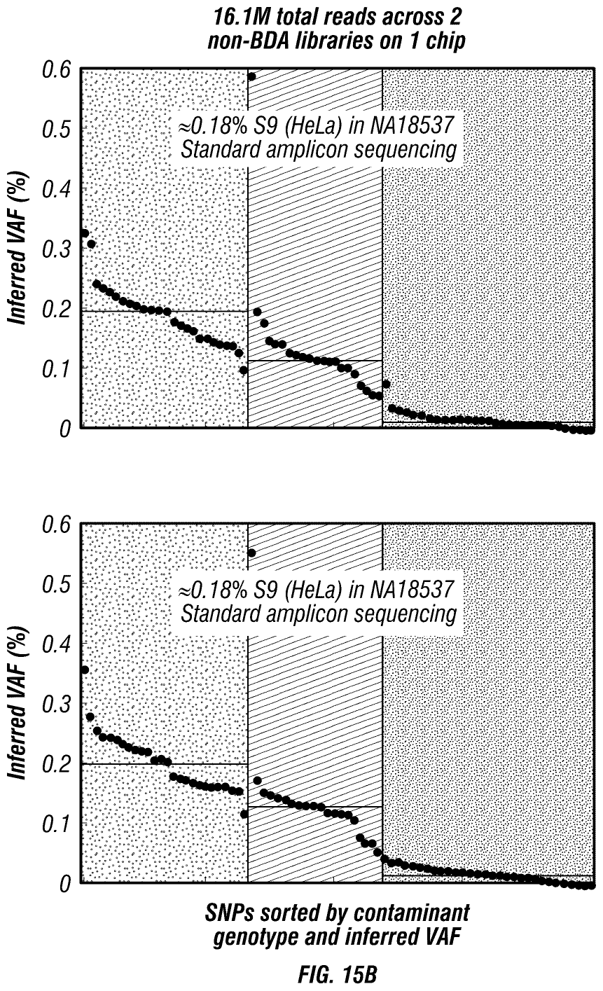

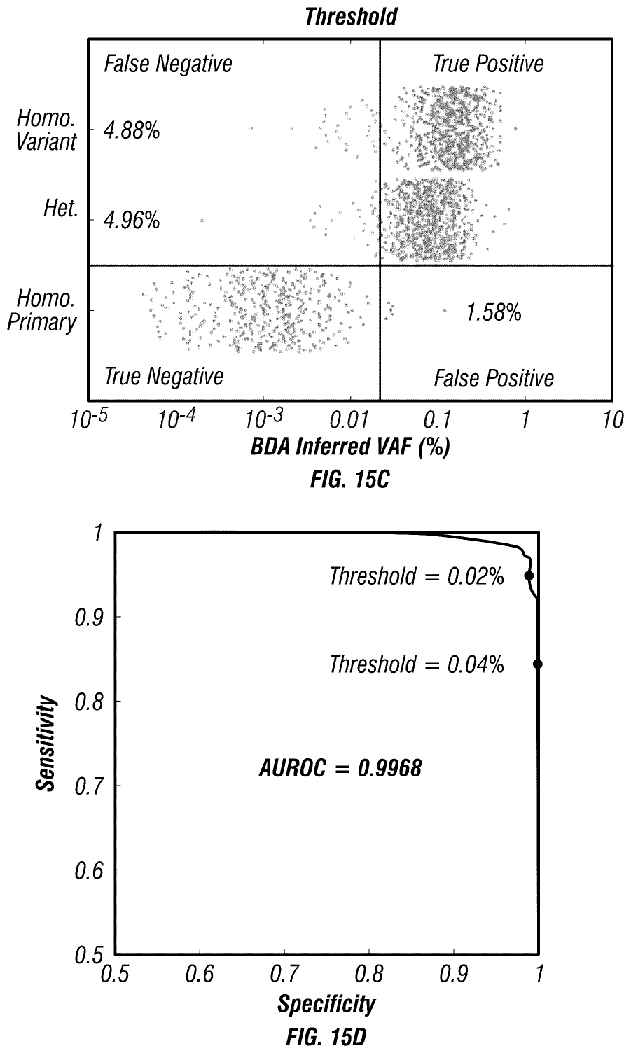

Results for 22 mBDA libraries on a single MiSeq chip are provided in FIG. 15A. Each library corresponds to NA18537 contaminated with between roughly 0.1% and 0.2% of a different human DNA. Each subfigure shows the inferred VAF for all 80 SNPs, sorted by contaminant genotype and then by inferred VAF. The black horizontal lines show the median inferred VAF for homozygous variants and heterozygous variants. For all 22 libraries, the median inferred VAF for homozygous primary alleles were below 0.01%. Comparison libraries using standard amplicon NGS on the samples contaminated with S9 (HeLa) are shown in FIG. 15B. Note the significantly higher inferred VAF for homozygous primary alleles. A summary of variant call accuracy using the 0.019% VAF LoD threshold described in FIG. 14E is shown in FIG. 15C. All inferred VAFs from panel (FIG. 15A) are displayed in this beeswarm plot. There is a false positive variant call rate of 1.58%, and a false negative rate of 4.88% or 4.96%, depending whether the unique contaminant allele is homozygous or heterozygous, respectively. A receiver operator characteristic (ROC) plot for variant calls using the data in panel (FIG. 15C) is shown in FIG. 15D. Setting the variant call threshold at 0.04% VAF would increase specificity to 100%, at the cost of reducing sensitivity to roughly 85%. The area under the ROC curve is very high at 0.9968.

### Example 9—Determination of Contaminant Identity Based on mBDA NGS Data

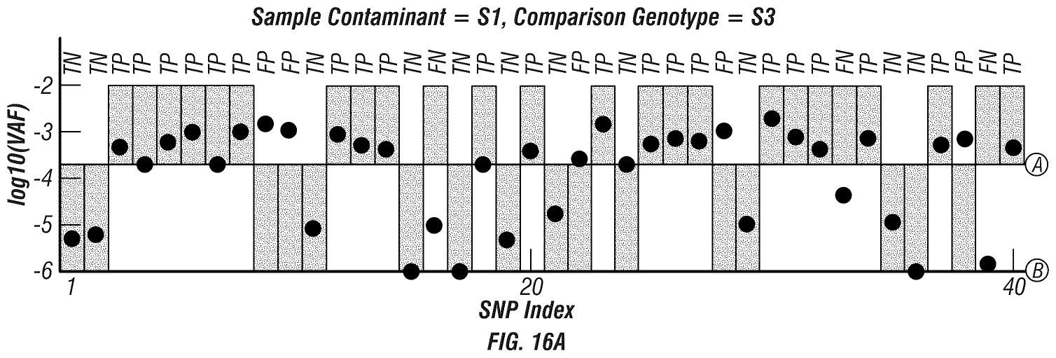

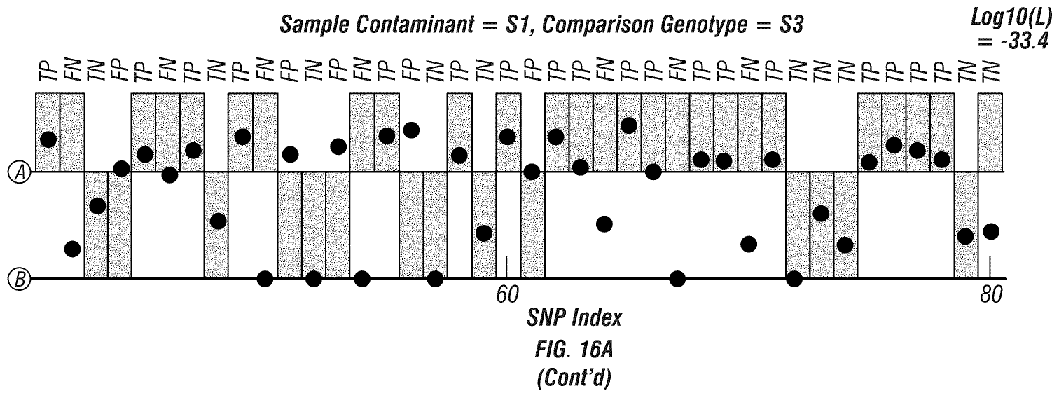

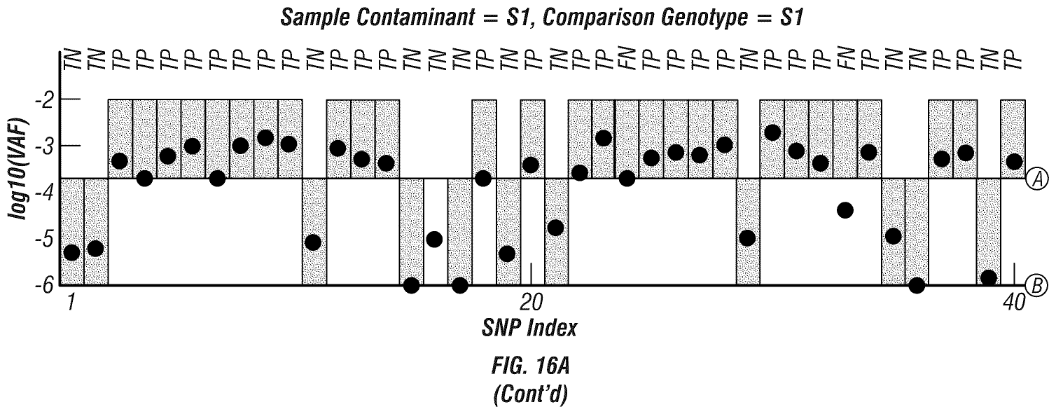

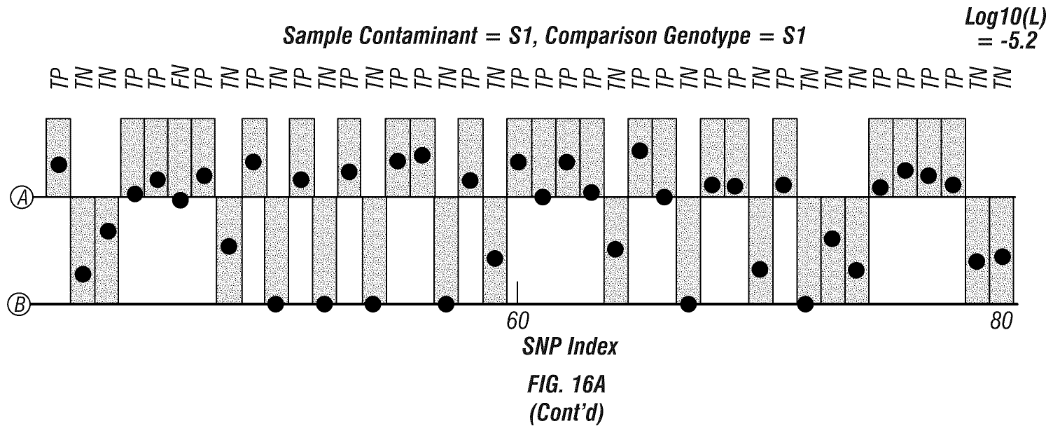

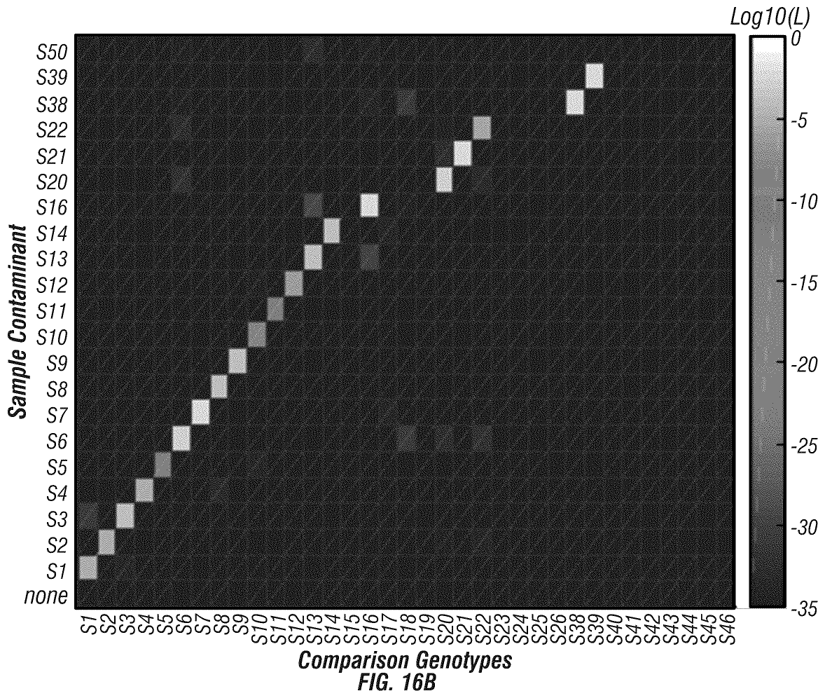

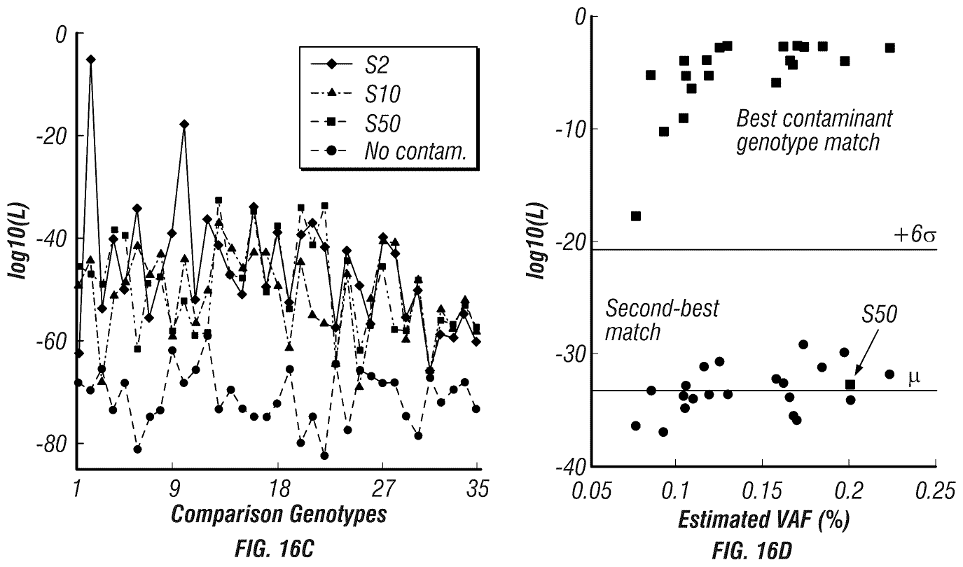

The calculation of contaminant likelihood based on mBDA variant calls is shown in FIG. 16A. For each SNP, a positive or negative variant allele call is made based on whether the inferred VAF (dots) is greater than the threshold of 0.019%. For each potential contaminant, whether a variant allele exists for each SNP (gray boxes) is considered, and thus whether the mBDA-inferred variant would be a True Positive (TP), True Negative (TN), False Positive (FP), or False Negative (FN) relative to the potential contaminant is determined. The overall likelihood L for the potential contaminant is calculated as the product of the likelihoods of all SNPs, with the likelihood of TP, TN, FP, and FN assigned as 95.08%, 98.42%, 1.58%, and 4.92%, respectively, based on FIG. 15C. The correct contaminant is expected to have a much higher value of L than incorrect contaminants. Heatmap plot of log 10(L) values for all pairwise combinations of all 22 tested samples (with contamination fraction between 0.07% and 0.22%) vs. 35 database genotypes is shown in FIG. 16B, based on data from FIG. 15A. Representative distributions of log 10(L) for 4 different samples are shown in FIG. 16C. S2 shows a typical sample whose contaminant genotype exists within the database. S10 shows the sample with the weakest maximum log 10(L) value whose genotype exists in the database. S50 shows a sample whose genotype is not included in the database of 35 genotypes. No contamination shows a sample of pure NA18537. A plot of highest and second-highest log 10(L) values against the contamination fraction is shown in FIG. 16D. The value of log 10(L) decreases for lower contamination fractions, consistent with expectations. However, even the S10 sample with 0.07% contamination is confidently identified, at over 6 standard deviations above the mean second-highest log 10(L) value. The S50 sample, which was not in the database, can also be confidently identified as an unknown contaminant based on its highest log 10(L) value.

### Example 10—Patterns of SNPs Across 85 SNP Loci for 23 Individuals

The patterns of SNPs across 85 SNP loci were analyzed for 23 individuals (volunteers from the Houston area). In the left panel of FIG. 9, each column corresponds to one individual. The top right panel of FIG. 9 shows the number of valid SNP alleles for detecting contamination assuming known base genotype, given every possible pair of genotypes from the left panel (23*22). The bottom right panel of FIG. 9 shows the number of valid SNP alleles for detecting contamination assuming known contaminant genotype (randomly selecting blockers for heterozygous loci), given every possible pair of genotypes from the left panel. In both cases, there is a significant number of valid SNP loci for detecting possible contamination.

TABLE 1
Sequences and concentrations of forward primers (fP),
blockers (B), and reverse primers (rP) used for the multiplex
BDA experiments with results shown in FIGs. 4, 6, and 10.
Note that the left panel of FIG. 4 did not use blockers.

| | | SEQ | | |
|---|---|---|---|---|
| | | ID | | |
| Name | Sequence | NO: | Conc. | |
| rs10230708_fP | ACCAATGGGAGTCACTGCTG | 1 | 15 | nM |
| rs10104396_fP | GAGGGGTATTAGAAGAATGACTATGTGA | 2 | 15 | nM |
| rs199032_fP | GCTCTTCCTCTCACATCTTTATTTAACC | 3 | 15 | nM |
| rs926850_fP | CAGAGTAAAATTTACTGCTCCGTCATAA | 4 | 15 | nM |
| rs17149369_fP | GGATTCCCTAAGCTCTTCAATATTGC | 5 | 15 | nM |
| rs869720_fP | CCTCATCTGTAAAGCAGGGAGAGA | 6 | 15 | nM |
| rs12478327_fP | ACTTCTGCCAACATTCAAATTCAGG | 7 | 15 | nM |
| rs2638145_fP | GGATGGGACTCCAATGCAAAACT | 8 | 15 | nM |
| rs2170091_fP | CATCTTGCTCTTCATAGATAGCTTCAGA | 9 | 15 | nM |
| rs2043583_fP | CCTGAATGTCAGTTTTGTTAGAGCAAC | 10 | 15 | nM |
| rs955456_fP | CAGACTTAATCAAAGCCCTTGAAAAGA | 11 | 15 | nM |
| rs966516_fP | CCTCCCATAGTGATTCTTATGAAGTCA | 12 | 15 | nM |
| rs354169_fP | AATGCTTTGCTTGCTGAGAACTT | 13 | 15 | nM |
| rs1898170_fP | AATGGGAAAACACATTTTAAGGGCA | 14 | 15 | nM |
| rs11247921_fP | CCACACTCTGCCTCTCATGGTAT | 15 | 15 | nM |
| rs1635718_fP | ACTTAAGAGGTCAACACAGATGAAAATTATC | 16 | 15 | nM |
| rs10510620_fP | TCCGCAAAACCTACAATCTCTGAA | 17 | 15 | nM |
| rs7104025_fP | TCAGATGCTTTAGGCTCATGAGTTA | 18 | 15 | nM |
| rs2246745_fP | CTCCTTGGAATCACCAACAAACAT | 19 | 15 | nM |
| rs3789806_fP | CTTGTATATAGACGGTAAAATAAACACCAAGA | 20 | 15 | nM |
| rs706714_fP | TGAAGCAGATGTTGAACAACAAGG | 21 | 15 | nM |
| rs1884444_fP | TTCCTGCTTCCAGACATGAATCA | 22 | 15 | nM |
| rs2510152_fP | ACCCAGGTGAGTTTTGTTTCACAT | 23 | 15 | nM |
| rs16754_fP | CTCTCTGCCTGCAGGATGTG | 24 | 15 | nM |
| rs206781_fP | CACTTCCTCCAGAAGGTCCAAAG | 25 | 15 | nM |
| rs28932178_fP | ACTAAGAGTGCAGAGCCTGGAA | 26 | 15 | nM |
| rs10186821_fP | GCGTTGTGCTGTCCATTGG | 27 | 15 | nM |
| rs10508599_fP | GGGTTAAAATCTTTTGCTTTCATATTGAGC | 28 | 15 | nM |
| rs10738578_fP | CCCGTTATATAAGAGGACATAATTGCAT | 29 | 15 | nM |
| rs10741037_fP | CACTTTATCAGACACAGTTATGTGCT | 30 | 15 | nM |
| rs10770674_fP | GCCCTATAGGTTTTCCTCCTACTGT | 31 | 15 | nM |
| rs10805227_fP | CTATCTGCAGGATTGTGTTCAATGTA | 32 | 15 | nM |
| rs10833604_fP | CTCTCTAGAGTGCAGATTGGTAGAA | 33 | 15 | nM |
| rs10964389_fP | CAAAGTTGATAAATTAAAGGACTAAGGCAC | 34 | 15 | nM |
| rs11015816_fP | CTGACCTAAGGCATGGGACTT | 35 | 15 | nM |
| rs11045749_fP | CATTCTGTCTGGGATGAGGTGAT | 36 | 15 | nM |
| rs1123828_fP | TGGAATCAAACATACTATGTGTCAAACA | 37 | 15 | nM |
| rs11708584_fP | GCGAAGTCATTTCGGTCCTCTTTAA | 38 | 15 | nM |
| rs12192635_fP | CCTCTGATTCCCAGACATAATGCT | 39 | 15 | nM |
| rs12213948_fP | TGAAAGACGTCACAGCAAGGT | 40 | 15 | nM |
| rs12259813_fP | TGTAGGAGAGATTGGGCTAGAGAG | 41 | 15 | nM |
| rs12541300_fP | ACAGAAACCAATTACCTATGAGGAGTAA | 42 | 15 | nM |
| rs12681931_fP | GAAAGTGGCACAGAAACTCAGAC | 43 | 15 | nM |
| rs12782580_fP | GCATTAGATCATTTAACACACAAAACCCTAT | 44 | 15 | nM |
| rs1375977_fP | TGCTCCTAAAAGCACCCAGC | 45 | 15 | nM |
| rs1516755_fP | CTAACTTCCTAACTAAAACTTTACAGTGGA | 46 | 15 | nM |
| rs1524303_fP | GGATTTCACACCCATTAGAATAACTACTAT | 47 | 15 | nM |
| rs1667087_fP | CCTCTAGAAAAAATGGAGATTTGGGAAT | 48 | 15 | nM |
| rs16871316_fP | GGACTTTTTTGCTTTTTGACACCTTTAC | 49 | 15 | nM |
| rs16925478_fP | ACGTATTTCTAACTATAGTGAGTGCATTATG | 50 | 15 | nM |
| rs17560702_fP | ACATGTCCAAAGAGAGAAGTCGTAG | 51 | 15 | nM |
| rs1937037_fP | GCACGTAGATGAAATTGCCCCATA | 52 | 15 | nM |
| rs2215492_fP | GCCCCAAAGGTTACCCCATG | 53 | 15 | nM |
| rs2301720_fP | GTAGCCGCTTCTCTGTGAGTT | 54 | 15 | nM |
| rs2616187_fP | GGAAAATATGTCTAAAAAGGCTCTGGAG | 55 | 15 | nM |
| rs2710998_fP | GTTTGTTCTAAGGTTCATCTGGTGAT | 56 | 15 | nM |
| rs2807238_fP | GTGGGCTTACATGATTGGATTAACTT | 57 | 15 | nM |
| rs2874755_fP | TGTCCCACTTTTTACCTCCCTTC | 58 | 15 | nM |
| rs3813787_fP | GGGCTTCGGAATCGGACTTG | 59 | 15 | nM |
| rs4665582_fP | TGTGCTACGACAGAGCTAAGTAC | 60 | 15 | nM |
| rs4712476_fP | CCCCGGATGTCAGGGAATG | 61 | 15 | nM |
| rs611628_fP | CCAGGCACCACTGCTTTGT | 62 | 15 | nM |
| rs6452035_fP | GCAGAAAAAAATGATATCTGAATTCTGGAT | 63 | 15 | nM |
| rs6816854_fP | CCTTTTTCACTGTTATGAAATGTACTTTCTT | 64 | 15 | nM |
| rs6937778_fP | AGGATGCTGGGGCTTGC | 65 | 15 | nM |
| rs7003044_fP | GTAAAGTGCATGGGGTCAAGTC | 66 | 15 | nM |
| rs7032336_fP | TGAGAAGTCTAACAAGTTAAATTCAGGAC | 67 | 15 | nM |
| rs7816009_fP | GGTAGAATGTTAGTGACTATGTACAATTTCA | 68 | 15 | nM |
| rs7893462_fP | ACCTTGTCAAGAACCTAAATAGTGAGAA | 69 | 15 | nM |
| rs7902135_fP | CGTGGGCTAGTCAAGAATATAAAATGTTAG | 70 | 15 | nM |
| rs898476_fP | CCTATATAGACTAATTTACTTAAACATTTAAA | 71 | 15 | nM |
| | CCCCA | | | |
| rs9368431_fP | GGTTCAACTCTCAGTTTTATTAGTTGTGT | 72 | 15 | nM |
| rs9438621_fP | AGCATCGTGAGGTTCTGAAAAGA | 73 | 15 | nM |
| rs9466035_fP | CCTAACACCAGTTCTTCCTCCAC | 74 | 15 | nM |
| rs9466930_fP | TGTGTGGCTCAGTATACCACTTAG | 75 | 15 | nM |
| rs9973865_fP | GAAAAAAAAGGGTCTCATTAGGAATCATTAC | 76 | 15 | nM |
| rs4712498_fP | GTTTTTATATGTTAGTGTCCCCATGGTATA | 77 | 15 | nM |
| rs2073149_fP | AGTGATCAGAAGGCTTTGATTTGA | 78 | 15 | nM |
| rs2862909_fP | GCACATCATACATTATTTCTGTTGCTAT | 79 | 15 | nM |
| rs1338945_fP | GAAATATTGCTGGGGTCAGCG | 80 | 15 | nM |
| rs10230708_B | ACTGCTGCAGGCGCCCTGT/iSpC3//iSpC3/TC | 81 | 150 | nM |
| rs10104396_B | GACTATGTGACAAAATAGCTAAGGATACAGGA | 82 | 150 | nM |
| | AATATG/iSpC3//iSpC3/GA | | | |
| rs199032_B | CATCTTTATTTAACCCATTAGAAAATCCTATC | 83 | 150 | nM |
| | AGCTCT/iSpC3//iSpC3/CG | | | |
| rs926850_B | CCGTCATAACAAAAACATATTTACTTTCTCTG | 84 | 150 | nM |
| | GC/iSpC3//iSpC3/CC | | | |
| rs17149369_B | CTTCAATATTGCAGAAGTGTTGCAAGCCl/iSpC3// | 85 | 150 | nM |
| | iSpC3/GT | | | |
| rs869720_B | AGGGAGAGAACCTCCTCCCTCACAGA/iSpC3// | 86 | 150 | nM |
| | iSpC3/TC | | | |
| rs12478327_B | TCAAATTCAGGTACCTTAGAGGGACAGCTAAA/ | 87 | 150 | nM |
| | iSpC3//iSpC3/CT | | | |
| rs2638145_B | AATGCAAAACTCAATGTATCAGTGTGAGGATGT/ | 88 | 150 | nM |
| | iSpC3//iSpC3/AT | | | |
| rs2170091_B | TAGCTTCAGAAACATTCCAGTGTATGTGCAG/ | 89 | 150 | nM |
| | iSpC3//iSpC3/GA | | | |
| rs2043583_B | GTTAGAGCAACTTTCCTTGATTCCCAGAGTAG/ | 90 | 150 | nM |
| | iSpC3//iSpC3/CT | | | |
| rs955456_B | CCTTGAAAAGAGGGCTTAGGTTTTCTTTGC/ | 91 | 150 | nM |
| | iSpC3//iSpC3/TA | | | |
| rs966516_B | CTTATGAAGTCATGGAACAATGCCTACTTCTA | 92 | 150 | nM |
| | TATTT/iSpC3//iSpC3/AG | | | |
| rs354169_B | CTGAGAACTTAGCATTAATTACCTTTTTTCAT | 93 | 150 | nM |
| | GAGAAT/iSpC3//iSpC3/TA | | | |
| rs1898170_B | AGGGCATTTTTTACAGTGTTGAATATTGAAAC | 94 | 150 | nM |
| | TG/iSpC3//iSpC3/TG | | | |
| rs11247921_B | CTCTCATGGTATGGTGTTTTTCTGTGCTCC/ | 95 | 150 | nM |
| | iSpC3//iSpC3/CG | | | |
| rs1635718_B | CAGATGAAAATTATCTGTGCTTTTTTGTAAGC | 96 | 150 | nM |
| | TGATATATT/iSpC3//iSpC3/TC | | | |
| rs10510620_B | CAATCTCTGAATCTCAGAATAGTAGCCTAGAA | 97 | 150 | nM |
| | AACG/iSpC3//iSpC3/GC | | | |
| rs7104025_B | CTCATGAGTTAACAAGGAGATGATGTAGTGTA | 98 | 150 | nM |
| | AAG/iSpC3//iSpC3/GC | | | |
| rs2246745_B | CAACAAACATGCCTTCTCCTTCTCCTGA/iSpC3// | 99 | 150 | nM |
| | iSpC3/AA | | | |
| rs3789806_B | TAAACACCAAGACGTGGTAAATATTTACCTGG | 100 | 150 | nM |
| | T/iSpC3//iSpC3/CG | | | |
| rs706714_B | CAACAAGGTCAGTATTGATAAGTGGTTGCT/ | 101 | 150 | nM |
| | iSpC3//iSpC3/AG | | | |
| rs1884444_B | ACATGAATCATGTCACTATTCAATGGGATGC/ | 102 | 150 | nM |
| | iSpC3//iSpC3/TT | | | |
| rs2510152_B | TTTTGTTTCACATGATAACCATATCACTGGAC | 103 | 150 | nM |
| | ACA/iSpC3//iSpC3/CC | | | |
| rs16754_B | AGGATGTGCGACGTGTGCCTG/iSpC3// | 104 | 150 | nM |
| | iSpC3/GG | | | |
| rs206781_B | GGTCCAAAGCCGGAAGGGCCTAAA/iSpC3// | 105 | 150 | nM |
| | iSpC3 /AA | | | |
| rs28932178_B | GCCTGGAACCGAGACGCCTCAG/iSpC3// | 106 | 150 | nM |
| | iSpC3/TG | | | |
| rs10186821_B | TCCATTGGCTACTCAGTCTCGGCT/iSpC3// | 107 | 150 | nM |
| | iSpC3/AT | | | |
| rs10508599_B | TCATATTGAGCTTAAGAGTTCAGAACACTGAT | 108 | 150 | nM |
| | GG/iSpC3//iSpC3/AT | | | |
| rs10738578_B | CATAATTGCATATAACCTACACACATTCTCCC | 109 | 150 | nM |
| | A/iSpC3//iSpC3/TT | | | |
| rs10741037_B | GTTATGTGCTGGAAAGAGCATAAATTTTGGAA | 110 | 150 | nM |
| | T/iSpC3//iSpC3/AA | | | |
| rs10770674_B | CTCCTACTGTACATACATATTATCTTAAGGAA | 111 | 150 | nM |
| | AAAATCCAAAT/iSpC3//iSpC3/AT | | | |
| rs10805227_B | TGTTCAATGTATTAAATAATCATCAGCATATT | 112 | 150 | nM |
| | TTTGTATTCAC/iSpC3//iSpC3/AA | | | |
| rs10833604_B | GATTGGTAGAAGACACTGATTGCATCTTCAA/ | 113 | 150 | nM |
| | iSpC3//iSpC3/GT | | | |
| rs10964389_B | AAGGCACAGAACAATCATGCAACTTGC/iSpC3// | 114 | 150 | nM |
| | iSpC3/AT | | | |
| rs11015816_B | GGGACTTTCTTGAGGGATGGCATCC/iSpC3// | 115 | 150 | nM |
| | iSpC3/CT | | | |
| rs11045749_B | GAGGTGATATCTCATTTTGGCTTCTATTTGCA/ | 116 | 150 | nM |
| | iSpC3//iSpC3/TA | | | |
| rs1123828_B | TGTCAAACACCCATGCTCACCCTT/iSpC3// | 117 | 150 | nM |
| | iSpC3/CA | | | |
| rs11708584_B | GGTCCTCTTTAAGGTCTCTACAATAAATTGCC | 118 | 150 | nM |
| | A/iSpC3//iSpC3/AA | | | |
| rs12192635_B | GACATAATGCTTTTGGTTGGACTTTCAAAAAG | 119 | 150 | nM |
| | G/iSpC3//iSpC3/GT | | | |
| rs12213948_B | GCAAGGTTCAAATCATTCTCTCCTATCTCATC/ | 120 | 150 | nM |
| | iSpC3//iSpC3/TT | | | |
| rs12259813_B | GCTAGAGAGATAATTGAGTGTCATCAGAACTA | 121 | 150 | nM |
| | GAT/iSpC3//iSpC3/TT | | | |
| rs12541300_B | ATGAGGAGTAATTGAAATCATTAATACCCACA | 122 | 150 | nM |
| | AACA/iSpC3//iSpC3/TT | | | |
| rs12681931_B | AACTCAGACCAATTTGGCCATAGATTATTAGC/ | 123 | 150 | nM |
| | iSpC3//iSpC3/TT | | | |
| rs12782580_B | ACAAAACCCTATAAGGAAGATGTCATTACCCA | 124 | 150 | nM |
| | TATTTTA/iSpC3//iSpC3/TT | | | |
| rs1375977_B | ACCCAGCTTTATACATTCACAAAGATATGGTT | 125 | 150 | nM |
| | TG/iSpC3//iSpC3/AA | | | |
| rs1516755_B | ACAGTGGAACAGCTCTCTCCTTCTTTTTT/ | 126 | 150 | nM |
| | iSpC3//iSpC3/CA | | | |
| rs1524303_B | ATTAGAATAACTACTATTAAAAAAACCCCACA | 127 | 150 | nM |
| | AAATAACTCTT/iSpC3//iSpC3/CT | | | |
| rs1667087_B | TTTGGGAATTAAAAGCCAATAGATTAGCTGAA | 128 | 150 | nM |
| | AATTC/iSpC3//iSpC3/AT | | | |
| rs16871316_B | ACACCTTTACATGAAGGCTTTGAAGTACTCTT/ | 129 | 150 | nM |
| | iSpC3//iSpC3/AT | | | |
| rs16925478_B | GTGCATTATGGGTAAGAATGTTCATTTATTAT | 130 | 150 | nM |
| | TTCACTTATA/iSpC3//iSpC3/GA | | | |
| rs17560702_B | GAAGTCGTAGCTATTCGGCAAAGGAAATG/ | 131 | 150 | nM |
| | iSpC3//iSpC3/TT | | | |
| rs1937037_B | TGCCCCATAGGCAGTGTTTGGTGAAG/iSpC3// | 132 | 150 | nM |
| | iSpC3/GT | | | |
| rs2215492_B | TACCCCATGTGTATCAAATGGTCAGCAAG/ | 133 | 150 | nM |
| | iSpC3//iSpC3/TT | | | |
| rs2301720_B | CTGTGAGTTGGGAGCAAAGGAGCA/iSpC3// | 134 | 150 | nM |
| | iSpC3/AT | | | |
| rs2616187_B | CTCTGGAGACGGGGGATGTTAAGTTGA/iSpC3// | 135 | 150 | nM |
| | iSpC3/AA | | | |
| rs2710998_B | TCTGGTGATTGAGAAAGCGTTCCAGA/iSpC3// | 136 | 150 | nM |
| | iSpC3/GA | | | |
| rs2807238_B | ATTGGATTAACTTTGGTGGAACCTACTTCGAT/ | 137 | 150 | nM |
| | iSpC3//iSpC3/AT | | | |
| rs2874755_B | CTCCCTTCTTTCATCCCTACATCATGTCC/ | 138 | 150 | nM |
| | iSpC3//iSpC3/AA | | | |
| rs3813787_B | CGGACTTGGCTGGGGTAGAGCTT/iSpC3// | 139 | 150 | nM |
| | iSpC3/AA | | | |
| rs4665582_B | GAGCTAAGTACCAGGTATGATGCTCGC/iSpC3// | 140 | 150 | nM |
| | iSpC3/AT | | | |
| rs4712476_B | AGGGAATGCTCTAGACAAAACACTGTTCC/ | 141 | 150 | nM |
| | iSpC3//iSpC3/TA | | | |
| rs611628_B | TGCTTTGTGCTAGCTCAAAGACTCACAT/ | 142 | 150 | nM |
| | iSpC3//iSpC3/TT | | | |
| rs6452035_B | AATTCTGGATCAAATTAAATATGTCGCATTCT | 143 | 150 | nM |
| | CC/iSpC3//iSpC3/GT | | | |
| rs6816854_B | TGTACTTTCTTTTTAGCCATAAGATGATTTCC | 144 | 150 | nM |
| | CAT/iSpC3//iSpC3/AT | | | |
| rs6937778_B | GCTTGCTTTCCCACACCACTACCT/iSpC3// | 145 | 150 | nM |
| | iSpC3/TA | | | |
| rs7003044_B | GGTCAAGTCTGAGGCTGTTGAGCTTA/iSpC3// | 146 | 150 | nM |
| | iSpC3/GA | | | |
| rs7032336_B | TTCAGGACGTGAAAGCACGAGAACG/iSpC3// | 147 | 150 | nM |
| | iSpC3 /AT | | | |
| rs7816009_B | ATGTACAATTTCAACTGGAGTTTCCATTGCA/ | 148 | 150 | nM |
| | iSpC3//iSpC3/GT | | | |
| rs7893462_B | AAATAGTGAGAACGAGCAGCTGCAGG/iSpC3// | 149 | 150 | nM |
| | iSpC3/CT | | | |
| rs7902135_B | AAGAATATAAAATGTTAGAGAACCACATACAA | 150 | 150 | nM |
| | CGAGC/iSpC3//iSpC3/CT | | | |
| rs898476_B | AACCCCAGAACACTAGCAGCTAAGGG/iSpC3// | 151 | 150 | nM |
| | iSpC3/TA | | | |
| rs9368431_B | TTTTATTAGTTGTGTAATCCAGTTACTTAACT | 152 | 150 | nM |
| | TTAAAAGCC/iSpC3//iSpC3/AT | | | |
| rs9438621_B | GTTCTGAAAAGAGCCTCCACTCCTGT/iSpC3// | 153 | 150 | nM |
| | iSpC3/TT | | | |
| rs9466035_B | CCTCCACTCCACCATGGCACCTATTA/iSpC3// | 154 | 150 | nM |
| | iSpC3/AA | | | |
| rs9466930_B | GTATACCACTTAGGCTATAGTTATTCTAAACT | 155 | 150 | nM |
| | TTGATAAAC/iSpC3//iSpC3/GT | | | |
| rs9973865_B | AGGAATCATTACAGGAAAACATCGTTTAAATT | 156 | 150 | nM |
| | GGA/iSpC3//iSpC3/AA | | | |
| rs4712498_B | CCATGGTATATTGTAAGTTGTAGGTACATACC | 157 | 150 | nM |
| | C/iSpC3//iSpC3/AA | | | |
| rs2073149_B | TTTGATTTGAATAAACCAGAGAACTCTTCTGA | 158 | 150 | nM |
| | G/iSpC3//iSpC3/TT | | | |
| rs2862909_B | TGTTGCTATCTTGCTTTTAGCATTTAGTGC/ | 159 | 150 | nM |
| | iSpC3//iSpC3/AA | | | |
| rs1338945_B | TCAGCGTTGAGTAATACCGTCTGCC/iSpC3// | 160 | 150 | nM |
| | iSpC3/CA | | | |
| rs10230708_rP | TAAGTGGAAAGAACTGGGGTGTC | 161 | 2.02 | nM |
| rs10104396_rP | ACATGGTTAGATATTAGCCTGACCTATG | 162 | 3.71 | nM |
| rs199032_rP | GCAGCCAAGTGTGAAAGTATTGA | 163 | 16.47 | nM |
| rs926850_rP | TGATGTTGAGTTGAGACAGGTTACA | 164 | 13.99 | nM |
| rs17149369_rP | AAATGTAGTTCTATTATGGTCAGCACAC | 165 | 7.77 | nM |
| rs869720_rP | AGTATCCCCAAAAGGTTGCAGAT | 166 | 6.23 | nM |
| rs12478327_rP | GTGCAAGCTGGAGGCACT | 167 | 3.23 | nM |
| rs2638145_rP | ACAGGAAAAGAAACTAAAATTGTACCCTT | 168 | 5.47 | nM |
| rs2170091_rP | GAAGCCAGATCTCAAAGTGTCCT | 169 | 5.17 | nM |
| rs2043583_rP | GTTATTGGGAATGCTATGAAAGAGACA | 170 | 7.73 | nM |
| rs955456_rP | AGAACTCATTTCCTTATAGCTGAAGAACT | 171 | 30.06 | nM |
| rs966516_rP | GCAGACACTTAGGATGTTTCCAGT | 172 | 2.61 | nM |
| rs354169_rP | GAGCCTTAGTTCCTCCATCAGTAAA | 173 | 8.31 | nM |
| rs1898170_rP | AAATTTACGTTGGTAATTGGGTCTTGT | 174 | 9.53 | nM |
| rs11247921_rP | CACAGAGGTGACAGAACACAGT | 175 | 8.00 | nM |
| rs1635718_rP | TAGTTATTCATGGTGGGAAGGCAA | 176 | 54.00 | nM |
| rs10510620_rP | AAAAGATAATGTTCTTGTTTATATGCCCTTG | 177 | 6.34 | nM |
| rs7104025_rP | TACAGCAACTCACAAACTAATGACTCT | 178 | 6.56 | nM |
| rs2246745_rP | GGCTGCGATGAGACAGGAA | 179 | 3.68 | nM |
| rs3789806_rP | AGGCACCAGAAGTCATCAGAATG | 180 | 9.35 | nM |
| rs706714_rP | GACCAAGCTTTTATGCACCACA | 181 | 3.92 | nM |
| rs1884444_rP | TGAAAGATAGCAATAGATACATAAAACACCA | 182 | 12.29 | nM |
| rs2510152_rP | TGAAACCACATACACACAAATTCACT | 183 | 4.07 | nM |
| rs16754_rP | CTTCCTGCTGTGCATCTGTAAGT | 184 | 16.06 | nM |
| rs206781_rP | AAAAAGAAGAAACGGAAGGCAGAG | 185 | 20.05 | nM |
| rs28932178_rP | TGCTGCCCCACCCTTTATTAAC | 186 | 2.38 | nM |
| rs10186821_rP | CCTATTGGAAGAACCTGCCAGAA | 187 | 2.92 | nM |
| rs10508599 rP | TGCAAAATGAAGCACAGCCC | 188 | 3.01 | nM |
| rs10738578_rP | GCAGATGGAAAATACTTGGGAAAAAAAT | 189 | 21.81 | nM |
| rs10741037_rP | GCAAAAATTACTATACCGACTTTAATAACGAAA | 190 | 27.73 | nM |
| rs10770674_rP | ACTCATTGTAGGCTGAACCTTGG | 191 | 3.31 | nM |
| rs10805227_rP | TGTATTGAGCATTTAGCACATGCC | 192 | 9.75 | nM |
| rs10833604_rP | CAATTTCCAAGACAGAAGCACTCC | 193 | 15.58 | nM |
| rs10964389_rP | ACTTACTGAGCACATGGCCTG | 194 | 2.20 | nM |
| rs11015816_rP | GGAGAGGGTGAGAAGTTGCAC | 195 | 1.74 | nM |
| rs11045749_rP | GGCAAAGACATTTTTCCAAGGAAGATAT | 196 | 4.40 | nM |
| rs1123828_rP | CACTGCCAGCTTGTGCCT | 197 | 6.66 | nM |
| rs11708584_rP | GCCCTAAATCCTAAATGAAATTGGCA | 198 | 2.97 | nM |
| rs12192635_rP | AGAGGAGAAATAGATGTAGCTGCC | 199 | 1.96 | nM |
| rs12213948_rP | AATCCAGTGACATTCTTTAAACTGTCTT | 200 | 7.64 | nM |
| rs12259813_rP | GCTGAGCTGTCACATCACTTCA | 201 | 2.72 | nM |
| rs12541300_rP | GCTGTGTAGCTTGGCAAATTAACTA | 202 | 13.62 | nM |
| rs12681931_rP | GCACTCTTGGGTAACAGGCTTT | 203 | 2.46 | nM |
| rs12782580_rP | CCATGCCCAGCCTGGC | 204 | 4.03 | nM |
| rs1375977_rP | TGGCTCCTCATAAGTTATGCAGATTT | 205 | 11.15 | nM |
| rs1516755_rP | CAGTAGGATTGGCTTTATCAAAGAGATC | 206 | 56.85 | nM |
| rs1524303_rP | ACCATAATGTTTTCCATAGAAGATGCAC | 207 | 18.98 | nM |
| rs1667087_rP | GGTTCTGTACTGAAGTAAAAATCTCATACTAT | 208 | 30.60 | nM |
| rs16871316_rP | GGCAAAGAAACATGGCAGAAATATCATA | 209 | 24.77 | nM |
| rs16925478_rP | CCTTTGGCATTTTGGTCAAGATTGT | 210 | 13.76 | nM |
| rs17560702_rP | GGGGGAAAATGGTTTCTTAGGATGA | 211 | 3.48 | nM |
| rs1937037_rP | CTCCCATTTTTCTAAGACATTTTTTTTTCTC | 212 | 9.86 | nM |
| rs2215492_rP | AGCATGCCGCCCTTGG | 213 | 1.82 | nM |
| rs2301720_rP | TCACAGGTCAAAATTATGAGTTCTTCG | 214 | 126.08 | nM |
| rs2616187_rP | TGAGAGTGTGCAAGTCACTTGT | 215 | 12.88 | nM |
| rs2710998_rP | GCAGGCAGCATGTATCCCAG | 216 | 4.92 | nM |
| rs2807238_rP | GTTTAATGGACAGTAGATGCTAAATTCTAGA | 217 | 12.28 | nM |
| rs2874755_rP | CGCCATAGTTAGCCGCTTCC | 218 | 3.33 | nM |
| rs3813787_rP | TGAGCCTCGGTCTCTACCTG | 219 | 30.51 | nM |
| rs4665582_rP | CCTTTAAGGCCCAGCAACTG | 220 | 3.56 | nM |
| rs4712476_rP | GGGTGACCTTTCCCTTTTGATGA | 221 | 4.37 | nM |
| rs611628_rP | TGTGTGTGAAAGCACTTTATAAACCA | 222 | 4.33 | nM |
| rs6452035_rP | CTATCCTCAGAATTTTCCATTGATACTAGAAATA | 223 | 70.00 | nM |
| rs6816854_fP | GAGTGTCTCCCAAACAAGGATCA | 224 | 126.08 | nM |
| rs6937778_rP | ACAGCCATCAGATATCCAGCAG | 225 | 1.44 | nM |
| rs7003044_rP | ACTTCGAGAATTGACTCTAAGTGGT | 226 | 4.68 | nM |
| rs7032336_rP | AATTTAGCTTCCTTGAGGATAGAAGTAAC | 227 | 25.22 | nM |
| rs7816009_rP | CCCGGCCACCCATACAG | 228 | 21.44 | nM |
| rs7893462_rP | GAAAACTACCTTAAACTATGTGAGAAAGAAC | 229 | 51.24 | nM |
| rs7902135_rP | ACCCTCACTAATCTTTTTCTGTTTGTTT | 230 | 3.01 | nM |
| rs898476_rP | GTTTTTCTCCCAGCTGTAAAAGCA | 231 | 39.43 | nM |
| rs9368431_rP | GCTTTAGTTTCTTTGCATATTTTCTGCAATA | 232 | 49.29 | nM |
| rs9438621_rP | AGCTGATCTGCAAGGTCTATTTGA | 233 | 21.17 | nM |
| rs9466035_rP | TGGGCTCAAGTGATCCACCTA | 234 | 2.93 | nM |
| rs9466930_rP | GTAAAGAGAAGGGCTACCAGGATTA | 235 | 4.97 | nM |
| rs9973865_rP | CCCTATGCCTGGGATACTTCCTT | 236 | 17.08 | nM |
| rs4712498_rP | ACAAATCTTTCATTTGTCTAAGGTATCAACT | 237 | 5.09 | nM |
| rs2073149_rP | AGTGTCTTGCATTTTCAAGTATTCCT | 238 | 7.02 | nM |
| rs2862909_rP | CCTAGGTTATTTGCTGTTCTCTTTCATTA | 239 | 4.55 | nM |
| rs1338945_rP | GCTTGCATATAGACCTACAAATACCACT | 240 | 3.40 | nM |

TABLE 2
Forward primers (fp) and reverse primers (rp) used
for analysis of the SNP alleles of 23 individuals shown in
FIG. 9. No blockers were used for this experiment.

| | | SEQ ID | |
|---|---|---|---|
| Name | Sequence | NO: | Conc. |
| rs10230708_fP | ACCAATGGGAGTCACTGCTG | 1 | 15 nM |
| rs10104396_fP | GAGGGGTATTAGAAGAATGACTATGTGA | 2 | 15 nM |
| rs199032_fP | GCTCTTCCTCTCACATCTTTATTTAACC | 3 | 15 nM |
| rs926850_fP | CAGAGTAAAATTTACTGCTCCGTCATAA | 4 | 15 nM |
| rs17149369_fP | GGATTCCCTAAGCTCTTCAATATTGC | 5 | 15 nM |
| rs869720_fP | CCTCATCTGTAAAGCAGGGAGAGA | 6 | 15 nM |
| rs12478327_fP | ACTTCTGCCAACATTCAAATTCAGG | 7 | 15 nM |
| rs2638145_fP | GGATGGGACTCCAATGCAAAACT | 8 | 15 nM |
| rs2170091_fP | CATCTTGCTCTTCATAGATAGCTTCAGA | 9 | 15 nM |
| rs2043583_fP | CCTGAATGTCAGTTTTGTTAGAGCAAC | 10 | 15 nM |
| rs955456_fP | CAGACTTAATCAAAGCCCTTGAAAAGA | 11 | 15 nM |
| rs966516_fP | CCTCCCATAGTGATTCTTATGAAGTCA | 12 | 15 nM |
| rs354169_fP | AATGCTTTGCTTGCTGAGAACTT | 13 | 15 nM |
| rs1898170_fP | AATGGGAAAACACATTTTAAGGGCA | 14 | 15 nM |
| rs11247921_fP | CCACACTCTGCCTCTCATGGTAT | 15 | 15 nM |
| rs10510620_fP | TCCGCAAAACCTACAATCTCTGAA | 17 | 15 nM |
| rs7104025_fP | TCAGATGCTTTAGGCTCATGAGTTA | 18 | 15 nM |
| rs2246745_fP | CTCCTTGGAATCACCAACAAACAT | 19 | 15 nM |
| rs3789806_fP | CTTGTATATAGACGGTAAAATAAACACCAAGA | 20 | 15 nM |
| rs706714_fP | TGAAGCAGATGTTGAACAACAAGG | 21 | 15 nM |
| rs1884444_fP | TTCCTGCTTCCAGACATGAATCA | 22 | 15 nM |
| rs2510152_fP | ACCCAGGTGAGTTTTGTTTCACAT | 23 | 15 nM |
| rs16754_fP | CTCTCTGCCTGCAGGATGTG | 24 | 15 nM |
| rs206781_fP | CACTTCCTCCAGAAGGTCCAAAG | 25 | 15 nM |
| rs28932178_fP | ACTAAGAGTGCAGAGCCTGGAA | 26 | 15 nM |
| rs10186821_fP | GCGTTGTGCTGTCCATTGG | 27 | 15 nM |
| rs10508599_fP | GGGTTAAAATCTTTTGCTTTCATATTGAGC | 28 | 15 nM |
| rs10738578_fP | CCCGTTATATAAGAGGACATAATTGCAT | 29 | 15 nM |
| rs10741037_fP | CACTTTATCAGACACAGTTATGTGCT | 30 | 15 nM |
| rs10770674_fP | GCCCTATAGGTTTTCCTCCTACTGT | 31 | 15 nM |
| rs10805227_fP | CTATCTGCAGGATTGTGTTCAATGTA | 32 | 15 nM |
| rs10833604_fP | CTCTCTAGAGTGCAGATTGGTAGAA | 33 | 15 nM |
| rs10964389_fP | CAAAGTTGATAAATTAAAGGACTAAGGCAC | 34 | 15 nM |
| rs11015816_fP | CTGACCTAAGGCATGGGACTT | 35 | 15 nM |
| rs11045749_fP | CATTCTGTCTGGGATGAGGTGAT | 36 | 15 nM |
| rs1123828_fP | TGGAATCAAACATACTATGTGTCAAACA | 37 | 15 nM |
| rs11573214_fP | GGCACCATGCATCCAGCC | 241 | 15 nM |
| rs11708584_fP | GCGAAGTCATTTCGGTCCTCTTTAA | 38 | 15 nM |
| rs12192635_fP | CCTCTGATTCCCAGACATAATGCT | 39 | 15 nM |
| rs12213948_fP | TGAAAGACGTCACAGCAAGGT | 40 | 15 nM |
| rs12259813_fP | TGTAGGAGAGATTGGGCTAGAGAG | 41 | 15 nM |
| rs12541300_fP | ACAGAAACCAATTACCTATGAGGAGTAA | 42 | 15 nM |
| rs12681931_fP | GAAAGTGGCACAGAAACTCAGAC | 43 | 15 nM |
| rs12782580_fP | GCATTAGATCATTTAACACACAAAACCCTAT | 44 | 15 nM |
| rs1375977_fP | TGCTCCTAAAAGCACCCAGC | 45 | 15 nM |
| rs1516755_fP | CTAACTTCCTAACTAAAACTTTACAGTGGA | 46 | 15 nM |
| rs1524303_fP | GGATTTCACACCCATTAGAATAACTACTAT | 47 | 15 nM |
| rs1667087_fP | CCTCTAGAAAAAATGGAGATTTGGGAAT | 48 | 15 nM |
| rs16871316_fP | GGACTTTTTTGCTTTTTGACACCTTTAC | 49 | 15 nM |
| rs16925478_fP | ACGTATTTCTAACTATAGTGAGTGCATTATG | 50 | 15 nM |
| rs17560702_fP | ACATGTCCAAAGAGAGAAGTCGTAG | 51 | 15 nM |
| rs1937037_fP | GCACGTAGATGAAATTGCCCCATA | 52 | 15 nM |
| rs2215492_fP | GCCCCAAAGGTTACCCCATG | 53 | 15 nM |
| rs2616187_fP | GGAAAATATGTCTAAAAAGGCTCTGGAG | 55 | 15 nM |
| rs2710998_fP | GTTTGTTCTAAGGTTCATCTGGTGAT | 56 | 15 nM |
| rs2807238_fP | GTGGGCTTACATGATTGGATTAACTT | 57 | 15 nM |
| rs2874755_fP | TGTCCCACTTTTTACCTCCCTTC | 58 | 15 nM |
| rs4665582_fP | TGTGCTACGACAGAGCTAAGTAC | 60 | 15 nM |
| rs4712476_fP | CCCCGGATGTCAGGGAATG | 61 | 15 nM |
| rs611628_fP | CCAGGCACCACTGCTTTGT | 62 | 15 nM |
| rs6452035_fP | GCAGAAAAAAATGATATCTGAATTCTGGAT | 63 | 15 nM |
| rs6816854_fP | CCTTTTTCACTGTTATGAAATGTACTTTCTT | 64 | 15 nM |
| rs6937778_fP | AGGATGCTGGGGCTTGC | 65 | 15 nM |
| rs7003044_fP | GTAAAGTGCATGGGGTCAAGTC | 66 | 15 nM |
| rs7032336_fP | TGAGAAGTCTAACAAGTTAAATTCAGGAC | 67 | 15 nM |
| rs7816009_fP | GGTAGAATGTTAGTGACTATGTACAATTTCA | 68 | 15 nM |
| rs7893462_fP | ACCTTGTCAAGAACCTAAATAGTGAGAA | 69 | 15 nM |
| rs7902135_fP | CGTGGGCTAGTCAAGAATATAAAATGTTAG | 70 | 15 nM |
| rs898476_fP | CCTATATAGACTAATTTACTTAAACATTTAA | 71 | 15 nM |
| | CCCCA | | |
| rs9368431_fP | GGTTCAACTCTCAGTTTTATTAGTTGTGT | 72 | 15 nM |
| rs9438621_fP | AGCATCGTGAGGTTCTGAAAAGA | 73 | 15 nM |
| rs9466035_fP | CCTAACACCAGTTCTTCCTCCAC | 74 | 15 nM |
| rs9466930_fP | TGTGTGGCTCAGTATACCACTTAG | 75 | 15 nM |
| rs9973865_fP | GAAAW1GGGTCTCATTAGGATCATTAC | 76 | 15 nM |
| rs4712498_fP | GTTTTTATATGTTAGTGTCCCCATGGTATA | 77 | 15 nM |
| rs2862909_fP | GCACATCATACATTATTTCTGTTGCTAT | 79 | 15 nM |
| rs1338945_fP | GAAATATTGCTGGGGTCAGCG | 80 | 15 nM |
| rs2301720_fP | GTAGCCGCTTCTCTGTGAGTT | 54 | 15 nM |
| rs955429_fP | GTCCAAGAGTGGAGGATTGGG | 242 | 15 nM |
| rs12095834_fP | GGAAATCGTACAGTTCCAAAGTACAA | 243 | 15 nM |
| rs10829268_fP | ACAGCAACAGAAACTACCCAAAAG | 244 | 15 nM |
| rs1635718_fP | ACTTAAGAGGTCAACACAGATGAAAATTATC | 16 | 15 nM |
| rs2073149_fP | AGTGATCAGAAGGCTTTGATTTGA | 78 | 15 nM |
| rs9358720_fP | CAAGCATCTTCTCCTTCCTCTCT | 245 | 15 nM |
| rs3813787_fP | GGGCTTCGGAATCGGACTTG | 59 | 15 nM |
| rs10230708_rP | TAAGTGGAAAGAACTGGGGTGTC | 161 | 15 nM |
| rs10104396_rP | ACATGGTTAGATATTAGCCTGACCTATG | 162 | 15 nM |
| rs199032_rP | GCAGCCAAGTGTGAAAGTATTGA | 163 | 15 nM |
| rs926850_rP | TGATGTTGAGTTGAGACAGGTTACA | 164 | 15 nM |
| rs17149369_rP | AAATGTAGTTCTATTATGGTCAGCACAC | 165 | 15 nM |
| rs869720_rP | AGTATCCCCAAAAGGTTGCAGAT | 166 | 15 nM |
| rs12478327_rP | GTGCAAGCTGGAGGCACT | 167 | 15 nM |
| rs2638145_rP | ACAGGAAAAGAAACTAAAATTGTACCCTT | 168 | 15 nM |
| rs2170091_rP | GAAGCCAGATCTCAAAGTGTCCT | 169 | 15 nM |
| rs2043583_rP | GTTATTGGGAATGCTATGAAAGAGACA | 170 | 15 nM |
| rs955456_rP | AGAACTCATTTCCTTATAGCTGAAGAACT | 171 | 15 nM |
| rs966516_rP | GCAGACACTTAGGATGTTTCCAGT | 172 | 15 nM |
| rs354169_rP | GAGCCTTAGTTCCTCCATCAGTAAA | 173 | 15 nM |
| rs1898170_rP | AAATTTACGTTGGTAATTGGGTCTTGT | 174 | 15 nM |
| rs11247921_rP | CACAGAGGTGACAGAACACAGT | 175 | 15 nM |
| rs10510620_rP | AAAAGATAATGTTCTTGTTTATATGCCCTTG | 177 | 15 nM |
| rs7104025_rP | TACAGCAACTCACAAACTAATGACTCT | 178 | 15 nM |
| rs2246745_rP | GGCTGCGATGAGACAGGAA | 179 | 15 nM |
| rs3789806_rP | AGGCACCAGAAGTCATCAGAATG | 180 | 15 nM |
| rs706714_rP | GACCAAGCTTTTATGCACCACA | 181 | 15 nM |
| rs1884444_rP | TGAAAGATAGCAATAGATACATAAAACACCA | 182 | 15 nM |
| rs2510152_rP | TGAAACCACATACACACAAATTCACT | 183 | 15 nM |
| rs16754_rP | CTTCCTGCTGTGCATCTGTAAGT | 184 | 15 nM |
| rs206781_rP | AAAAAGAAGAAACGGAAGGCAGAG | 185 | 15 nM |
| rs28932178_rP | TGCTGCCCCACCCTTTATTAAC | 186 | 15 nM |
| rs10186821_rP | CCTATTGGAAGAACCTGCCAGAA | 187 | 15 nM |
| rs10508599_rP | TGCAAAATGAAGCACAGCCC | 188 | 15 nM |
| rs10738578_rP | GCAGATGGAAAATACTTGGGAAAAAAAT | 189 | 15 nM |
| rs10741037_rP | GCAAAAATTACTATACCGACTTTAATAACGAAA | 190 | 15 nM |
| rs10770674_rP | ACTCATTGTAGGCTGAACCTTGG | 191 | 15 nM |
| rs10805227_rP | TGTATTGAGCATTTAGCACATGCC | 192 | 15 nM |
| rs10833604_rP | CAATTTCCAAGACAGAAGCACTCC | 193 | 15 nM |
| rs10964389_rP | ACTTACTGAGCACATGGCCTG | 194 | 15 nM |
| rs11015816_rP | GGAGAGGGTGAGAAGTTGCAC | 195 | 15 nM |
| rs11045749_rP | GGCAAAGACATTTTTCCAAGGAAGATAT | 196 | 15 nM |
| rs1123828_rP | CACTGCCAGCTTGTGCCT | 197 | 15 nM |
| rs11573214_rP | CCTAGTCCTTAACCACTCCTTACAG | 246 | 15 nM |
| rs11708584_rP | GCCCTAAATCCTAAATGAAATTGGCA | 198 | 15 nM |
| rs12192635_rP | AGAGGAGAAATAGATGTAGCTGCC | 199 | 15 nM |
| rs12213948_rP | AATCCAGTGACATTCTTTAAACTGTCTT | 200 | 15 nM |
| rs12259813_rP | GCTGAGCTGTCACATCACTTCA | 201 | 15 nM |
| rs12541300_rP | GCTGTGTAGCTTGGCAAATTAACTA | 202 | 15 nM |
| rs12681931_rP | GCACTCTTGGGTAACAGGCTTT | 203 | 15 nM |
| rs12782580_rP | CCATGCCCAGCCTGGC | 204 | 15 nM |
| rs1375977_rP | TGGCTCCTCATAAGTTATGCAGATTT | 205 | 15 nM |
| rs1516755_rP | CAGTAGGATTGGCTTTATCAAAGAGATC | 206 | 15 nM |
| rs1524303_rP | ACCATAATGTTTTCCATAGAAGATGCAC | 207 | 15 nM |
| rs1667087_rP | GGTTCTGTACTGAAGTAAAAATCTCATACTAT | 208 | 15 nM |
| rs16871316_rP | GGCAAAGAAACATGGCAGAAATATCATA | 209 | 15 nM |
| rs16925478_rP | CCTTTGGCATTTTGGTCAAGATTGT | 210 | 15 nM |
| rs17560702_rP | GGGGGAAAATGGTTTCTTAGGATGA | 211 | 15 nM |
| rs1937037_rP | CTCCCATTTTTCTAAGACATTTTTTTTTCTC | 212 | 15 nM |
| rs2215492_rP | AGCATGCCGCCCTTGG | 213 | 15 nM |
| rs2616187_rP | TGAGAGTGTGCAAGTCACTTGT | 215 | 15 nM |
| rs2710998_rP | GCAGGCAGCATGTATCCCAG | 216 | 15 nM |
| rs2807238_rP | GTTTAATGGACAGTAGATGCTAAATTCTAGA | 217 | 15 nM |
| rs2874755_rP | CGCCATAGTTAGCCGCTTCC | 218 | 15 nM |
| rs4665582_rP | CCTTTAAGGCCCAGCAACTG | 220 | 15 nM |
| rs4712476_rP | GGGTGACCTTTCCCTTTTGATGA | 221 | 15 nM |
| rs611628_rP | TGTGTGTGAAAGCACTTTATAAACCA | 222 | 15 nM |
| rs6452035_rP | CTATCCTCAGAATTTTCCATTGATACTAGAAATA | 223 | 15 nM |
| rs6816854_rP | GAGTGTCTCCCAAACAAGGATCA | 224 | 15 nM |
| rs6937778_rP | ACAGCCATCAGATATCCAGCAG | 225 | 15 nM |
| rs7003044_rP | ACTTCGAGAATTGACTCTAAGTGGT | 226 | 15 nM |
| rs7032336_rP | AATTTAGCTTCCTTGAGGATAGAAGTAAC | 227 | 15 nM |
| rs7816009_rP | CCCGGCCACCCATACAG | 228 | 15 nM |
| rs7893462_rP | GAAAACTACCTTAAACTATGTGAGAAAGAAC | 229 | 15 nM |
| rs7902135_rP | ACCCTCACTAATCTTTTTCTGTTTGTTT | 230 | 15 nM |
| rs898476_rP | GTTTTTCTCCCAGCTGTAAAAGCA | 231 | 15 nM |
| rs9368431_rP | GCTTTAGTTTCTTTGCATATTTTCTGCAATA | 232 | 15 nM |
| rs9438621_rP | AGCTGATCTGCAAGGTCTATTTGA | 233 | 15 nM |
| rs9466035_rP | TGGGCTCAAGTGATCCACCTA | 234 | 15 nM |
| rs9466930_rP | GTAAAGAGAAGGGCTACCAGGATTA | 235 | 15 nM |
| rs9973865_rP | CCCTATGCCTGGGATACTTCCTT | 236 | 15 nM |
| rs4712498_rP | ACAAATCTTTCATTTGTCTAAGGTATCAACT | 237 | 15 nM |
| rs2862909_rP | CCTAGGTTATTTGCTGTTCTCTTTCATTA | 239 | 15 nM |
| rs1338945_rP | GCTTGCATATAGACCTACAAATACCACT | 240 | 15 nM |
| rs2301720_rP | TCACAGGTCAAAATTATGAGTTCTTCG | 214 | 15 nM |
| rs955429_rP | TCCAAACTGGAAATGGCTGTATCT | 247 | 15 nM |
| rs12095834_rP | GCTTTCTATTTATTTAAAAGAAAGTGAAGTCCC | 248 | 15 nM |
| rs10829268_rP | TTTCCCCCCTTCTCTCTTCTTTTT | 249 | 15 nM |
| rs1635718_rP | TAGTTATTCATGGTGGGAAGGCAA | 176 | 15 nM |
| rs2073149_rP | AGTGTCTTGCATTTTCAAGTATTCCT | 238 | 15 nM |
| rs9358720_rP | CAGCAGTACACTGAACAGAATCC | 250 | 15 nM |
| rs3813787_rP | TGAGCCTCGGTCTCTACCTG | 219 | 15 nM |

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Patent Appln. Publn. No. 2017/0067090
SantaLucia & Hicks, “The thermodynamics of DNA structural motifs,” Annu. Rev. Biophys. Biomol. Struct., 33:415-440 (2004).

1-53. (canceled)

54. A panel of nonpathogenic SNPs comprising at least 30 nonpathogenic SNPs, wherein each SNP has an alternative allele with a population frequency of between 10% and 90%, wherein each pair of SNPs is either on different chromosomes or has a genomic distance of at least 2,000 nucleotides, wherein the sequence 50 nucleotides upstream and 50 nucleotides downstream of the SNP is unique within the organism's genome.

55. The panel of claim 54, wherein the panel is for use in verifying the genomic identity of an individual or an organism.

56. The panel of claim 54, wherein the sequence 50 nucleotides upstream and 50 nucleotides downstream of the SNP are unique within the organism's genome if no other region of the organism's genome has a greater than 90% homology to the sequence.

57. The panel of claim 54, wherein each SNP has an alternative allele with a population frequency of between 20% and 80%.

58. The panel of claim 54, wherein the organism is Homo sapiens.

59. The panel of claim 58, wherein the panel comprises SNPs from each of the 22 pairs of autosomes in the human genome.

60. (canceled)

61. The panel of claim 54, wherein the panel comprises at least 80 nonpathogenic SNPs.

62. The panel of claim 54, wherein the panel comprises at least 30 nonpathogenic SNPs selected from the group consisting of: rs10230708; rs10104396; rs199032; rs926850; rs17149369; rs869720; rs12478327; rs2638145; rs2170091; rs2043583; rs955456; rs966516; rs354169; rs1898170; rs11247921; rs10510620; rs7104025; rs2246745; rs3789806; rs706714; rs1884444; rs2510152; rs16754; rs206781; rs28932178; rs10186821; rs10508599; rs10738578; rs10741037; rs10770674; rs10805227; rs10833604; rs10964389; rs11015816; rs11045749; rs1123828; rs11573214; rs11708584; rs12192635; rs12213948; rs12259813; rs12541300; rs12681931; rs12782580; rs1375977; rs1516755; rs1524303; rs1667087; rs16871316; rs16925478; rs17560702; rs1937037; rs2215492; rs2616187; rs2710998; rs2807238; rs2874755; rs4665582; rs4712476; rs611628; rs6452035; rs6816854; rs6937778; rs7003044; rs7032336; rs7816009; rs7893462; rs7902135; rs898476; rs9368431; rs9438621; rs9466035; rs9466930; rs9973865; rs4712498; rs2862909; rs1338945; rs2301720; rs955429; rs12095834; rs10829268; rs1635718; rs2073149; rs9358720; and rs3813787.

63. The panel of claim 54, wherein the panel comprises the following nonpathogenic SNPs: rs10230708; rs10104396; rs199032; rs926850; rs17149369; rs869720; rs12478327; rs2638145; rs2170091; rs2043583; rs955456; rs966516; rs354169; rs1898170; rs11247921; rs10510620; rs7104025; rs2246745; rs3789806; rs706714; rs1884444; rs2510152; rs16754; rs206781; rs28932178; rs10186821; rs10508599; rs10738578; rs10741037; rs10770674; rs10805227; rs10833604; rs10964389; rs11015816; rs11045749; rs1123828; rs11573214; rs11708584; rs12192635; rs12213948; rs12259813; rs12541300; rs12681931; rs12782580; rs1375977; rs1516755; rs1524303; rs1667087; rs16871316; rs16925478; rs17560702; rs1937037; rs2215492; rs2616187; rs2710998; rs2807238; rs2874755; rs4665582; rs4712476; rs611628; rs6452035; rs6816854; rs6937778; rs7003044; rs7032336; rs7816009; rs7893462; rs7902135; rs898476; rs9368431; rs9438621; rs9466035; rs9466930; rs9973865; rs4712498; rs2862909; rs1338945; rs2301720; rs955429; rs12095834; rs10829268; rs1635718; rs2073149; rs9358720; and rs3813787.

64. The panel of claim 54, wherein the panel comprises at least 30 nonpathogenic SNPs selected from the group consisting of: rs10230708; rs10104396; rs199032; rs926850; rs17149369; rs869720; rs12478327; rs2638145; rs2170091; rs2043583; rs955456; rs966516; rs354169; rs1898170; rs11247921; rs1635718; rs10510620; rs7104025; rs2246745; rs3789806; rs706714; rs1884444; rs2510152; rs16754; rs206781; rs28932178; rs10186821; rs10508599; rs10738578; rs10741037; rs10770674; rs10805227; rs10833604; rs10964389; rs11015816; rs11045749; rs1123828; rs11708584; rs12192635; rs12213948; rs12259813; rs12541300; rs12681931; rs12782580; rs1375977; rs1516755; rs1524303; rs1667087; rs16871316; rs16925478; rs17560702; rs1937037; rs2215492; rs2301720; rs2616187; rs2710998; rs2807238; rs2874755; rs3813787; rs4665582; rs4712476; rs611628; rs6452035; rs6816854; rs6937778; rs7003044; rs7032336; rs7816009; rs7893462; rs7902135; rs898476; rs9368431; rs9438621; rs9466035; rs9466930; rs9973865; rs4712498; rs2073149; rs2862909; and rs1338945.

65. The panel of claim 54, wherein the panel comprises the following nonpathogenic SNPs: rs10230708; rs10104396; rs199032; rs926850; rs17149369; rs869720; rs12478327; rs2638145; rs2170091; rs2043583; rs955456; rs966516; rs354169; rs1898170; rs11247921; rs1635718; rs10510620; rs7104025; rs2246745; rs3789806; rs706714; rs1884444; rs2510152; rs16754; rs206781; rs28932178; rs10186821; rs10508599; rs10738578; rs10741037; rs10770674; rs10805227; rs10833604; rs10964389; rs11015816; rs11045749; rs1123828; rs11708584; rs12192635; rs12213948; rs12259813; rs12541300; rs12681931; rs12782580; rs1375977; rs1516755; rs1524303; rs1667087; rs16871316; rs16925478; rs17560702; rs1937037; rs2215492; rs2301720; rs2616187; rs2710998; rs2807238; rs2874755; rs3813787; rs4665582; rs4712476; rs611628; rs6452035; rs6816854; rs6937778; rs7003044; rs7032336; rs7816009; rs7893462; rs7902135; rs898476; rs9368431; rs9438621; rs9466035; rs9466930; rs9973865; rs4712498; rs2073149; rs2862909; and rs1338945.


SEQUENCE LISTING (252 sequences)

SEQ ID NO: 1 - DNA, 20 bp, synthetic construct
  misc_feature 1..20: note = Synthetic polynucleotide
  source 1..20: mol_type = other DNA
accaatgggagtcactgctg

SEQ ID NO: 2 - DNA, 28 bp, synthetic construct
  misc_feature 1..28: note = Synthetic polynucleotide
  source 1..28: mol_type = other DNA
gaggggtattagaagaatgactatgtga

SEQ ID NO: 3 - DNA, 28 bp, synthetic construct
  misc_feature 1..28: note = Synthetic polynucleotide
  source 1..28: mol_type = other DNA
gctcttcctctcacatctttatttaacc

SEQ ID NO: 4 - DNA, 28 bp, synthetic construct
  misc_feature 1..28: note = Synthetic polynucleotide
  source 1..28: mol_type = other DNA
cagagtaaaatttactgctccgtcataa

SEQ ID NO: 5 - DNA, 26 bp, synthetic construct
  misc_feature 1..26: note = Synthetic polynucleotide
  source 1..26: mol_type = other DNA
ggattccctaagctcttcaatattgc

SEQ ID NO: 6 - DNA, 24 bp, synthetic construct
  misc_feature 1..24: note = Synthetic polynucleotide
  source 1..24: mol_type = other DNA
cctcatctgtaaagcagggagaga

SEQ ID NO: 7 - DNA, 25 bp, synthetic construct
  misc_feature 1..25: note = Synthetic polynucleotide
  source 1..25: mol_type = other DNA
acttctgccaacattcaaattcagg

SEQ ID NO: 8 - DNA, 23 bp, synthetic construct
  misc_feature 1..23: note = Synthetic polynucleotide
  source 1..23: mol_type = other DNA
ggatgggactccaatgcaaaact

SEQ ID NO: 9 - DNA, 28 bp, synthetic construct
  misc_feature 1..28: note = Synthetic polynucleotide
  source 1..28: mol_type = other DNA
catcttgctcttcatagatagcttcaga

SEQ ID NO: 10 - DNA, 27 bp, synthetic construct
  misc_feature 1..27: note = Synthetic polynucleotide
  source 1..27: mol_type = other DNA
cctgaatgtcagttttgttagagcaac

SEQ ID NO: 11 - DNA, 27 bp, synthetic construct
  misc_feature 1..27: note = Synthetic polynucleotide
  source 1..27: mol_type = other DNA
cagacttaatcaaagcccttgaaaaga

SEQ ID NO: 12 - DNA, 27 bp, synthetic construct
  misc_feature 1..27: note = Synthetic polynucleotide
  source 1..27: mol_type = other DNA
cctcccatagtgattcttatgaagtca

SEQ ID NO: 13 - DNA, 23 bp, synthetic construct
  misc_feature 1..23: note = Synthetic polynucleotide
  source 1..23: mol_type = other DNA
aatgctttgcttgctgagaactt

SEQ ID NO: 14 - DNA, 25 bp, synthetic construct
  misc_feature 1..25: note = Synthetic polynucleotide
  source 1..25: mol_type = other DNA
aatgggaaaacacattttaagggca

SEQ ID NO: 15 - DNA, 23 bp, synthetic construct
  misc_feature 1..23: note = Synthetic polynucleotide
  source 1..23: mol_type = other DNA
ccacactctgcctctcatggtat

SEQ ID NO: 16 - DNA, 31 bp, synthetic construct
  misc_feature 1..31: note = Synthetic polynucleotide
  source 1..31: mol_type = other DNA
acttaagaggtcaacacagatgaaaattatc

SEQ ID NO: 17 - DNA, 24 bp, synthetic construct
  misc_feature 1..24: note = Synthetic polynucleotide
  source 1..24: mol_type = other DNA
tccgcaaaacctacaatctctgaa

SEQ ID NO: 18 - DNA, 25 bp, synthetic construct
  misc_feature 1..25: note = Synthetic polynucleotide
  source 1..25: mol_type = other DNA
tcagatgctttaggctcatgagtta

SEQ ID NO: 19 - DNA, 24 bp, synthetic construct
  misc_feature 1..24: note = Synthetic polynucleotide
  source 1..24: mol_type = other DNA
ctccttggaatcaccaacaaacat

SEQ ID NO: 20 - DNA, 32 bp, synthetic construct
  misc_feature 1..32: note = Synthetic polynucleotide
  source 1..32: mol_type = other DNA
cttgtatatagacggtaaaataaacaccaaga

SEQ ID NO: 21 - DNA, 24 bp, synthetic construct
  misc_feature 1..24: note = Synthetic polynucleotide
  source 1..24: mol_type = other DNA
tgaagcagatgttgaacaacaagg

SEQ ID NO: 22 - DNA, 23 bp, synthetic construct
  misc_feature 1..23: note = Synthetic polynucleotide
  source 1..23: mol_type = other DNA
ttcctgcttccagacatgaatca

SEQ ID NO: 23 - DNA, 24 bp, synthetic construct
  misc_feature 1..24: note = Synthetic polynucleotide
  source 1..24: mol_type = other DNA
acccaggtgagttttgtttcacat

SEQ ID NO: 24 - DNA, 20 bp, synthetic construct
  misc_feature 1..20: note = Synthetic polynucleotide
  source 1..20: mol_type = other DNA
ctctctgcctgcaggatgtg

SEQ ID NO: 25 - DNA, 23 bp, synthetic construct
  misc_feature 1..23: note = Synthetic polynucleotide
  source 1..23: mol_type = other DNA
cacttcctccagaaggtccaaag

SEQ ID NO: 26 - DNA, 22 bp, synthetic construct
  misc_feature 1..22: note = Synthetic polynucleotide
  source 1..22: mol_type = other DNA
actaagagtgcagagcctggaa

SEQ ID NO: 27 - DNA, 19 bp, synthetic construct
  misc_feature 1..19: note = Synthetic polynucleotide
  source 1..19: mol_type = other DNA
gcgttgtgctgtccattgg

SEQ ID NO: 28 - DNA, 30 bp, synthetic construct
  misc_feature 1..30: note = Synthetic polynucleotide
  source 1..30: mol_type = other DNA
gggttaaaatcttttgctttcatattgagc

SEQ ID NO: 29 - DNA, 28 bp, synthetic construct
  misc_feature 1..28: note = Synthetic polynucleotide
  source 1..28: mol_type = other DNA
cccgttatataagaggacataattgcat

SEQ ID NO: 30 - DNA, 26 bp, synthetic construct
  misc_feature 1..26: note = Synthetic polynucleotide
  source 1..26: mol_type = other DNA
cactttatcagacacagttatgtgct

SEQ ID NO: 31 - DNA, 25 bp, synthetic construct
  misc_feature 1..25: note = Synthetic polynucleotide
  source 1..25: mol_type = other DNA
gccctataggttttcctcctactgt

SEQ ID NO: 32 - DNA, 26 bp, synthetic construct
  misc_feature 1..26: note = Synthetic polynucleotide
  source 1..26: mol_type = other DNA
ctatctgcaggattgtgttcaatgta

SEQ ID NO: 33 - DNA, 25 bp, synthetic construct
  misc_feature 1..25: note = Synthetic polynucleotide
  source 1..25: mol_type = other DNA
ctctctagagtgcagattggtagaa

SEQ ID NO: 34 - DNA, 30 bp, synthetic construct
  misc_feature 1..30: note = Synthetic polynucleotide
  source 1..30: mol_type = other DNA
caaagttgataaattaaaggactaaggcac

SEQ ID NO: 35 - DNA, 21 bp, synthetic construct
  misc_feature 1..21: note = Synthetic polynucleotide
  source 1..21: mol_type = other DNA
ctgacctaaggcatgggactt

SEQ ID NO: 36 - DNA, 23 bp, synthetic construct
  misc_feature 1..23: note = Synthetic polynucleotide
  source 1..23: mol_type = other DNA
cattctgtctgggatgaggtgat

SEQ ID NO: 37 - DNA, 28 bp, synthetic construct
  misc_feature 1..28: note = Synthetic polynucleotide
  source 1..28: mol_type = other DNA
tggaatcaaacatactatgtgtcaaaca

SEQ ID NO: 38 - DNA, 25 bp, synthetic construct
  misc_feature 1..25: note = Synthetic polynucleotide
  source 1..25: mol_type = other DNA
gcgaagtcatttcggtcctctttaa

SEQ ID NO: 39 - DNA, 24 bp, synthetic construct
  misc_feature 1..24: note = Synthetic polynucleotide
  source 1..24: mol_type = other DNA
cctctgattcccagacataatgct

SEQ ID NO: 40 - DNA, 21 bp, synthetic construct
  misc_feature 1..21: note = Synthetic polynucleotide
  source 1..21: mol_type = other DNA
tgaaagacgtcacagcaaggt

SEQ ID NO: 41 - DNA, 24 bp, synthetic construct
  misc_feature 1..24: note = Synthetic polynucleotide
  source 1..24: mol_type = other DNA
tgtaggagagattgggctagagag

SEQ ID NO: 42 - DNA, 28 bp, synthetic construct
  misc_feature 1..28: note = Synthetic polynucleotide
  source 1..28: mol_type = other DNA
acagaaaccaattacctatgaggagtaa

SEQ ID NO: 43 - DNA, 23 bp, synthetic construct
  misc_feature 1..23: note = Synthetic polynucleotide
  source 1..23: mol_type = other DNA
gaaagtggcacagaaactcagac

SEQ ID NO: 44 - DNA, 31 bp, synthetic construct
  misc_feature 1..31: note = Synthetic polynucleotide
  source 1..31: mol_type = other DNA
gcattagatcatttaacacacaaaaccctat

SEQ ID NO: 45 - DNA, 20 bp, synthetic construct
  misc_feature 1..20: note = Synthetic polynucleotide
  source 1..20: mol_type = other DNA
tgctcctaaaagcacccagc

SEQ ID NO: 46 - DNA, 30 bp, synthetic construct
  misc_feature 1..30: note = Synthetic polynucleotide
  source 1..30: mol_type = other DNA
ctaacttcctaactaaaactttacagtgga

SEQ ID NO: 47 - DNA, 30 bp, synthetic construct
  misc_feature 1..30: note = Synthetic polynucleotide
  source 1..30: mol_type = other DNA
ggatttcacacccattagaataactactat

SEQ ID NO: 48 - DNA, 28 bp, synthetic construct
  misc_feature 1..28: note = Synthetic polynucleotide
  source 1..28: mol_type = other DNA
cctctagaaaaaatggagatttgggaat

SEQ ID NO: 49 - DNA, 28 bp, synthetic construct
  misc_feature 1..28: note = Synthetic polynucleotide
  source 1..28: mol_type = other DNA
ggacttttttgctttttgacacctttac

SEQ ID NO: 50 - DNA, 31 bp, synthetic construct
  misc_feature 1..31: note = Synthetic polynucleotide
  source 1..31: mol_type = other DNA
acgtatttctaactatagtgagtgcattatg

SEQ ID NO: 51 - DNA, 25 bp, synthetic construct
  misc_feature 1..25: note = Synthetic polynucleotide
  source 1..25: mol_type = other DNA
acatgtccaaagagagaagtcgtag

SEQ ID NO: 52 - DNA, 24 bp, synthetic construct
  misc_feature 1..24: note = Synthetic polynucleotide
  source 1..24: mol_type = other DNA
gcacgtagatgaaattgccccata

SEQ ID NO: 53 - DNA, 20 bp, synthetic construct
  misc_feature 1..20: note = Synthetic polynucleotide
  source 1..20: mol_type = other DNA
gccccaaaggttaccccatg

SEQ ID NO: 54 - DNA, 21 bp, synthetic construct
  misc_feature 1..21: note = Synthetic polynucleotide
  source 1..21: mol_type = other DNA
gtagccgcttctctgtgagtt

SEQ ID NO: 55 - DNA, 28 bp, synthetic construct
  misc_feature 1..28: note = Synthetic polynucleotide
  source 1..28: mol_type = other DNA
ggaaaatatgtctaaaaaggctctggag

SEQ ID NO: 56 - DNA, 26 bp, synthetic construct
  misc_feature 1..26: note = Synthetic polynucleotide
  source 1..26: mol_type = other DNA
gtttgttctaaggttcatctggtgat

SEQ ID NO: 57 - DNA, 26 bp, synthetic construct
  misc_feature 1..26: note = Synthetic polynucleotide
  source 1..26: mol_type = other DNA
gtgggcttacatgattggattaactt

SEQ ID NO: 58 - DNA, 23 bp, synthetic construct
  misc_feature 1..23: note = Synthetic polynucleotide
  source 1..23: mol_type = other DNA
tgtcccactttttacctcccttc

SEQ ID NO: 59 - DNA, 20 bp, synthetic construct
  misc_feature 1..20: note = Synthetic polynucleotide
  source 1..20: mol_type = other DNA
gggcttcggaatcggacttg

SEQ ID NO: 60 - DNA, 23 bp, synthetic construct
  misc_feature 1..23: note = Synthetic polynucleotide
  source 1..23: mol_type = other DNA
tgtgctacgacagagctaagtac

SEQ ID NO: 61 - DNA, 19 bp, synthetic construct
  misc_feature 1..19: note = Synthetic polynucleotide
  source 1..19: mol_type = other DNA
ccccggatgtcagggaatg

SEQ ID NO: 62 - DNA, 19 bp, synthetic construct
  misc_feature 1..19: note = Synthetic polynucleotide
  source 1..19: mol_type = other DNA
ccaggcaccactgctttgt

SEQ ID NO: 63 - DNA, 30 bp, synthetic construct
  misc_feature 1..30: note = Synthetic polynucleotide
  source 1..30: mol_type = other DNA
gcagaaaaaaatgatatctgaattctggat

SEQ ID NO: 64 - DNA, 31 bp, synthetic construct
  misc_feature 1..31: note = Synthetic polynucleotide
  source 1..31: mol_type = other DNA
cctttttcactgttatgaaatgtactttctt

SEQ ID NO: 65 - DNA, 17 bp, synthetic construct
  misc_feature 1..17: note = Synthetic polynucleotide
  source 1..17: mol_type = other DNA
aggatgctggggcttgc

SEQ ID NO: 66 - DNA, 22 bp, synthetic construct
  misc_feature 1..22: note = Synthetic polynucleotide
  source 1..22: mol_type = other DNA
gtaaagtgcatggggtcaagtc

SEQ ID NO: 67 - DNA, 29 bp, synthetic construct
  misc_feature 1..29: note = Synthetic polynucleotide
  source 1..29: mol_type = other DNA
tgagaagtctaacaagttaaattcaggac

SEQ ID NO: 68 - DNA, 31 bp, synthetic construct
  misc_feature 1..31: note = Synthetic polynucleotide
  source 1..31: mol_type = other DNA
ggtagaatgttagtgactatgtacaatttca

SEQ ID NO: 69 - DNA, 28 bp, synthetic construct
  misc_feature 1..28: note = Synthetic polynucleotide
  source 1..28: mol_type = other DNA
accttgtcaagaacctaaatagtgagaa

SEQ ID NO: 70 - DNA, 30 bp, synthetic construct
  misc_feature 1..30: note = Synthetic polynucleotide
  source 1..30: mol_type = other DNA
cgtgggctagtcaagaatataaaatgttag

SEQ ID NO: 71 - DNA, 37 bp, synthetic construct
  misc_feature 1..37: note = Synthetic polynucleotide
  source 1..37: mol_type = other DNA
cctatatagactaatttacttaaacatttaaacccca

SEQ ID NO: 72 - DNA, 29 bp, synthetic construct
  misc_feature 1..29: note = Synthetic polynucleotide
  source 1..29: mol_type = other DNA
ggttcaactctcagttttattagttgtgt

SEQ ID NO: 73 - DNA, 23 bp, synthetic construct
  misc_feature 1..23: note = Synthetic polynucleotide
  source 1..23: mol_type = other DNA
agcatcgtgaggttctgaaaaga

SEQ ID NO: 74 - DNA, 23 bp, synthetic construct
  misc_feature 1..23: note = Synthetic polynucleotide
  source 1..23: mol_type = other DNA
cctaacaccagttcttcctccac

SEQ ID NO: 75 - DNA, 24 bp, synthetic construct
  misc_feature 1..24: note = Synthetic polynucleotide
  source 1..24: mol_type = other DNA
tgtgtggctcagtataccacttag

SEQ ID NO: 76 - DNA, 31 bp, synthetic construct
  misc_feature 1..31: note = Synthetic polynucleotide
  source 1..31: mol_type = other DNA
gaaaaaaaagggtctcattaggaatcattac

SEQ ID NO: 77 - DNA, 30 bp, synthetic construct
  misc_feature 1..30: note = Synthetic polynucleotide
  source 1..30: mol_type = other DNA
gtttttatatgttagtgtccccatggtata

SEQ ID NO: 78 - DNA, 24 bp, synthetic construct
  misc_feature 1..24: note = Synthetic polynucleotide
  source 1..24: mol_type = other DNA
agtgatcagaaggctttgatttga

SEQ ID NO: 79 - DNA, 28 bp, synthetic construct
  misc_feature 1..28: note = Synthetic polynucleotide
  source 1..28: mol_type = other DNA
gcacatcatacattatttctgttgctat

SEQ ID NO: 80 - DNA, 21 bp, synthetic construct
  misc_feature 1..21: note = Synthetic polynucleotide
  source 1..21: mol_type = other DNA
gaaatattgctggggtcagcg

SEQ ID NO: 81 - DNA, 23 bp, synthetic construct
  misc_feature 1..23: note = Synthetic polynucleotide
  misc_feature 20..21: note = n = any nucleotide, present or absent
  source 1..23: mol_type = other DNA
actgctgcaggcgccctgtnntc

SEQ ID NO: 82 - DNA, 42 bp, synthetic construct
  misc_feature 1..42: note = Synthetic polynucleotide
  misc_feature 39..40: note = n = any nucleotide, present or absent
  source 1..42: mol_type = other DNA
gactatgtgacaaaatagctaaggatacaggaaatatgnnga

SEQ ID NO: 83 - DNA, 42 bp, synthetic construct
  misc_feature 1..42: note = Synthetic polynucleotide
  misc_feature 39..40: note = n = any nucleotide, present or absent
  source 1..42: mol_type = other DNA
catctttatttaacccattagaaaatcctatcagctctnncg

SEQ ID NO: 84 - DNA, 38 bp, synthetic construct
  misc_feature 1..38: note = Synthetic polynucleotide
  misc_feature 35..36: note = n = any nucleotide, present or absent
  source 1..38: mol_type = other DNA
ccgtcataacaaaaacatatttactttctctggcnncc

SEQ ID NO: 85 - DNA, 33 bp, synthetic construct
  misc_feature 1..33: note = Synthetic polynucleotide
  misc_feature 30..31: note = n = any nucleotide, present or absent
  source 1..33: mol_type = other DNA
cttcaatattgcagaagtgttgcaagcctnngt

SEQ ID NO: 86 - DNA, 30 bp, synthetic construct
  misc_feature 1..30: note = Synthetic polynucleotide
  misc_feature 27..28: note = n = any nucleotide, present or absent
  source 1..30: mol_type = other DNA
agggagagaacctcctccctcacaganntc

SEQ ID NO: 87 - DNA, 36 bp, synthetic construct
  misc_feature 1..36: note = Synthetic polynucleotide
  misc_feature 33..34: note = n = any nucleotide, present or absent
  source 1..36: mol_type = other DNA
tcaaattcaggtaccttagagggacagctaaannct

SEQ ID NO: 88 - DNA, 37 bp, synthetic construct
  misc_feature 1..37: note = Synthetic polynucleotide
  misc_feature 34..35: note = n = any nucleotide, present or absent
  source 1..37: mol_type = other DNA
aatgcaaaactcaatgtatcagtgtgaggatgtnnat

SEQ ID NO: 89 - DNA, 35 bp, synthetic construct
  misc_feature 1..35: note = Synthetic polynucleotide
  misc_feature 32..33: note = n = any nucleotide, present or absent
  source 1..35: mol_type = other DNA
tagcttcagaaacattccagtgtatgtgcagnnga

SEQ ID NO: 90 - DNA, 36 bp, synthetic construct
  misc_feature 1..36: note = Synthetic polynucleotide
  misc_feature 33..34: note = n = any nucleotide, present or absent
  source 1..36: mol_type = other DNA
gttagagcaactttccttgattcccagagtagnnct

SEQ ID NO: 91 - DNA, 34 bp, synthetic construct
  misc_feature 1..34: note = Synthetic polynucleotide
  misc_feature 31..32: note = n = any nucleotide, present or absent
  source 1..34: mol_type = other DNA
ccttgaaaagagggcttaggttttctttgcnnta

SEQ ID NO: 92 - DNA, 41 bp, synthetic construct
  misc_feature 1..41: note = Synthetic polynucleotide
  misc_feature 38..39: note = n = any nucleotide, present or absent
  source 1..41: mol_type = other DNA
cttatgaagtcatggaacaatgcctacttctatatttnnag

SEQ ID NO: 93 - DNA, 42 bp, synthetic construct
  misc_feature 1..42: note = Synthetic polynucleotide
  misc_feature 39..40: note = n = any nucleotide, present or absent
  source 1..42: mol_type = other DNA
ctgagaacttagcattaattaccttttttcatgagaatnnta

SEQ ID NO: 94 - DNA, 38 bp, synthetic construct
  misc_feature 1..38: note = Synthetic polynucleotide
  misc_feature 35..36: note = n = any nucleotide, present or absent
  source 1..38: mol_type = other DNA
agggcattttttacagtgttgaatattgaaactgnntg

SEQ ID NO: 95 - DNA, 34 bp, synthetic construct
  misc_feature 1..34: note = Synthetic polynucleotide
  misc_feature 31..32: note = n = any nucleotide, present or absent
  source 1..34: mol_type = other DNA
ctctcatggtatggtgtttttctgtgctccnncg

SEQ ID NO: 96 - DNA, 45 bp, synthetic construct
  misc_feature 1..45: note = Synthetic polynucleotide
  misc_feature 42..43: note = n = any nucleotide, present or absent
  source 1..45: mol_type = other DNA
cagatgaaaattatctgtgcttttttgtaagctgatatattnntc

SEQ ID NO: 97 - DNA, 40 bp, synthetic construct
  misc_feature 1..40: note = Synthetic polynucleotide
  misc_feature 37..38: note = n = any nucleotide, present or absent
  source 1..40: mol_type = other DNA
caatctctgaatctcagaatagtagcctagaaaacgnngc

SEQ ID NO: 98 - DNA, 39 bp, synthetic construct
  misc_feature 1..39: note = Synthetic polynucleotide
  misc_feature 36..37: note = n = any nucleotide, present or absent
  source 1..39: mol_type = other DNA
ctcatgagttaacaaggagatgatgtagtgtaaagnngc

SEQ ID NO: 99 - DNA, 32 bp, synthetic construct
  misc_feature 1..32: note = Synthetic polynucleotide
  misc_feature 29..30: note = n = any nucleotide, present or absent
  source 1..32: mol_type = other DNA
caacaaacatgccttctccttctcctgannaa

SEQ ID NO: 100 - DNA, 37 bp, synthetic construct
  misc_feature 1..37: note = Synthetic polynucleotide
  misc_feature 34..35: note = n = any nucleotide, present or absent
  source 1..37: mol_type = other DNA
taaacaccaagacgtggtaaatatttacctggtnncg

SEQ ID NO: 101 - DNA, 34 bp, synthetic construct
  misc_feature 1..34: note = Synthetic polynucleotide
  misc_feature 31..32: note = n = any nucleotide, present or absent
  source 1..34: mol_type = other DNA
caacaaggtcagtattgataagtggttgctnnag

SEQ ID NO: 102 - DNA, 35 bp, synthetic construct
  misc_feature 1..35: note = Synthetic polynucleotide
  misc_feature 32..33: note = n = any nucleotide, present or absent
  source 1..35: mol_type = other DNA
acatgaatcatgtcactattcaatgggatgcnntt

SEQ ID NO: 103 - DNA, 39 bp, synthetic construct
  misc_feature 1..39: note = Synthetic polynucleotide
  misc_feature 36..37: note = n = any nucleotide, present or absent
  source 1..39: mol_type = other DNA
ttttgtttcacatgataaccatatcactggacacanncc

SEQ ID NO: 104 - DNA, 25 bp, synthetic construct
  misc_feature 1..25: note = Synthetic polynucleotide
  misc_feature 22..23: note = n = any nucleotide, present or absent
  source 1..25: mol_type = other DNA
aggatgtgcgacgtgtgcctgnngg

SEQ ID NO: 105 - DNA, 28 bp, synthetic construct
  misc_feature 1..28: note = Synthetic polynucleotide
  misc_feature 25..26: note = n = any nucleotide, present or absent
  source 1..28: mol_type = other DNA
ggtccaaagccggaagggcctaaannaa

SEQ ID NO: 106 - DNA, 26 bp, synthetic construct
  misc_feature 1..26: note = Synthetic polynucleotide
  misc_feature 23..24: note = n = any nucleotide, present or absent
  source 1..26: mol_type = other DNA
gcctggaaccgagacgcctcagnntg

SEQ ID NO: 107 - DNA, 28 bp, synthetic construct
  misc_feature 1..28: note = Synthetic polynucleotide
  misc_feature 25..26: note = n = any nucleotide, present or absent
  source 1..28: mol_type = other DNA
tccattggctactcagtctcggctnnat

SEQ ID NO: 108 - DNA, 38 bp, synthetic construct
  misc_feature 1..38: note = Synthetic polynucleotide
  misc_feature 35..36: note = n = any nucleotide, present or absent
  source 1..38: mol_type = other DNA
tcatattgagcttaagagttcagaacactgatggnnat

SEQ ID NO: 109 - DNA, 37 bp, synthetic construct
  misc_feature 1..37: note = Synthetic polynucleotide
  misc_feature 34..35: note = n = any nucleotide, present or absent
  source 1..37: mol_type = other DNA
cataattgcatataacctacacacattctcccanntt

SEQ ID NO: 110 - DNA, 37 bp, synthetic construct
  misc_feature 1..37: note = Synthetic polynucleotide
  misc_feature 34..35: note = n = any nucleotide, present or absent
  source 1..37: mol_type = other DNA
gttatgtgctggaaagagcataaattttggaatnnaa

SEQ ID NO: 111 - DNA, 47 bp, synthetic construct
  misc_feature 1..47: note = Synthetic polynucleotide
  misc_feature 44..45: note = n = any nucleotide, present or absent
  source 1..47: mol_type = other DNA
ctcctactgtacatacatattatcttaaggaaaaaatccaaatnnat

SEQ ID NO: 112 - DNA, 47 bp, synthetic construct
  misc_feature 1..47: note = Synthetic polynucleotide
  misc_feature 44..45: note = n = any nucleotide, present or absent
  source 1..47: mol_type = other DNA
tgttcaatgtattaaataatcatcagcatatttttgtattcacnnaa

SEQ ID NO: 113 - DNA, 35 bp, synthetic construct
  misc_feature 1..35: note = Synthetic polynucleotide
  misc_feature 32..33: note = n = any nucleotide, present or absent
  source 1..35: mol_type = other DNA
gattggtagaagacactgattgcatcttcaanngt

SEQ ID NO: 114 - DNA, 31 bp, synthetic construct
  misc_feature 1..31: note = Synthetic polynucleotide
  misc_feature 28..29: note = n = any nucleotide, present or absent
  source 1..31: mol_type = other DNA
aaggcacagaacaatcatgcaacttgcnnat

SEQ ID NO: 115 - DNA, 29 bp, synthetic construct
  misc_feature 1..29: note = Synthetic polynucleotide
  misc_feature 26..27: note = n = any nucleotide, present or absent
  source 1..29: mol_type = other DNA
gggactttcttgagggatggcatccnnct

SEQ ID NO: 116 - DNA, 36 bp, synthetic construct
  misc_feature 1..36: note = Synthetic polynucleotide
  misc_feature 33..34: note = n = any nucleotide, present or absent
  source 1..36: mol_type = other DNA
gaggtgatatctcattttggcttctatttgcannta

SEQ ID NO: 117 - DNA, 28 bp, synthetic construct
  misc_feature 1..28: note = Synthetic polynucleotide
  misc_feature 25..26: note = n = any nucleotide, present or absent
  source 1..28: mol_type = other DNA
tgtcaaacacccatgctcacccttnnca

SEQ ID NO: 118 - DNA, 37 bp, synthetic construct
  misc_feature 1..37: note = Synthetic polynucleotide
  misc_feature 34..35: note = n = any nucleotide, present or absent
  source 1..37: mol_type = other DNA
ggtcctctttaaggtctctacaataaattgccannaa

SEQ ID NO: 119 - DNA, 37 bp, synthetic construct
  misc_feature 1..37: note = Synthetic polynucleotide
  misc_feature 34..35: note = n = any nucleotide, present or absent
  source 1..37: mol_type = other DNA
gacataatgcttttggttggactttcaaaaaggnngt

SEQ ID NO: 120 - DNA, 36 bp, synthetic construct
  misc_feature 1..36: note = Synthetic polynucleotide
  misc_feature 33..34: note = n = any nucleotide, present or absent
  source 1..36: mol_type = other DNA
gcaaggttcaaatcattctctcctatctcatcnntt

SEQ ID NO: 121 - DNA, 39 bp, synthetic construct
  misc_feature 1..39: note = Synthetic polynucleotide
  misc_feature 36..37: note = n = any nucleotide, present or absent
  source 1..39: mol_type = other DNA
gctagagagataattgagtgtcatcagaactagatnntt

SEQ ID NO: 122 - DNA, 40 bp, synthetic construct
  misc_feature 1..40: note = Synthetic polynucleotide
  misc_feature 37..38: note = n = any nucleotide, present or absent
  source 1..40: mol_type = other DNA
atgaggagtaattgaaatcattaatacccacaaacanntt

SEQ ID NO: 123 - DNA, 36 bp, synthetic construct
  misc_feature 1..36: note = Synthetic polynucleotide
  misc_feature 33..34: note = n = any nucleotide, present or absent
  source 1..36: mol_type = other DNA
aactcagaccaatttggccatagattattagcnntt

SEQ ID NO: 124 - DNA, 43 bp, synthetic construct
  misc_feature 1..43: note = Synthetic polynucleotide
  misc_feature 40..41: note = n = any nucleotide, present or absent
  source 1..43: mol_type = other DNA
acaaaaccctataaggaagatgtcattacccatattttanntt

SEQ ID NO: 125 - DNA, 38 bp, synthetic construct
  misc_feature 1..38: note = Synthetic polynucleotide
  misc_feature 35..36: note = n = any nucleotide, present or absent
  source 1..38: mol_type = other DNA
acccagctttatacattcacaaagatatggtttgnnaa

SEQ ID NO: 126 - DNA, 33 bp, synthetic construct
  misc_feature 1..33: note = Synthetic polynucleotide
  misc_feature 30..31: note = n = any nucleotide, present or absent
  source 1..33: mol_type = other DNA
acagtggaacagctctctccttcttttttnnca

SEQ ID NO: 127 - DNA, 47 bp, synthetic construct
  misc_feature 1..47: note = Synthetic polynucleotide
  misc_feature 44..45: note = n = any nucleotide, present or absent
  source 1..47: mol_type = other DNA
attagaataactactattaaaaaaaccccacaaaataactcttnnct

SEQ ID NO: 128 - DNA, 41 bp, synthetic construct
  misc_feature 1..41: note = Synthetic polynucleotide
  misc_feature 38..39: note = n = any nucleotide, present or absent
  source 1..41: mol_type = other DNA
tttgggaattaaaagccaatagattagctgaaaattcnnat

SEQ ID NO: 129 - DNA, 36 bp, synthetic construct
  misc_feature 1..36: note = Synthetic polynucleotide
  misc_feature 33..34: note = n = any nucleotide, present or absent
  source 1..36: mol_type = other DNA
acacctttacatgaaggctttgaagtactcttnnat

SEQ ID NO: 130 - DNA, 46 bp, synthetic construct
  misc_feature 1..46: note = Synthetic polynucleotide
  misc_feature 43..44: note = n = any nucleotide, present or absent
  source 1..46: mol_type = other DNA
gtgcattatgggtaagaatgttcatttattatttcacttatannga

SEQ ID NO: 131 - DNA, 33 bp, synthetic construct
  misc_feature 1..33: note = Synthetic polynucleotide
  misc_feature 30..31: note = n = any nucleotide, present or absent
  source 1..33: mol_type = other DNA
gaagtcgtagctattcggcaaaggaaatgnntt

SEQ ID NO: 132 - DNA, 30 bp, synthetic construct
  misc_feature 1..30: note = Synthetic polynucleotide
  misc_feature 27..28: note = n = any nucleotide, present or absent
  source 1..30: mol_type = other DNA
tgccccataggcagtgtttggtgaagnngt

SEQ ID NO: 133 - DNA, 33 bp, synthetic construct
  misc_feature 1..33: note = Synthetic polynucleotide
  misc_feature 30..31: note = n = any nucleotide, present or absent
  source 1..33: mol_type = other DNA
taccccatgtgtatcaaatggtcagcaagnntt

SEQ ID NO: 134 - DNA, 28 bp, synthetic construct
  misc_feature 1..28: note = Synthetic polynucleotide
  misc_feature 25..26: note = n = any nucleotide, present or absent
  source 1..28: mol_type = other DNA
ctgtgagttgggagcaaaggagcannat

SEQ ID NO: 135 - DNA, 31 bp, synthetic construct
  misc_feature 1..31: note = Synthetic polynucleotide
  misc_feature 28..29: note = n = any nucleotide, present or absent
  source 1..31: mol_type = other DNA
ctctggagacgggggatgttaagttgannaa

SEQ ID NO: 136 - DNA, 30 bp, synthetic construct
  misc_feature 1..30: note = Synthetic polynucleotide
  misc_feature 27..28: note = n = any nucleotide, present or absent
  source 1..30: mol_type = other DNA
tctggtgattgagaaagcgttccagannga

SEQ ID NO: 137 - DNA, 36 bp, synthetic construct
  misc_feature 1..36: note = Synthetic polynucleotide
  misc_feature 33..34: note = n = any nucleotide, present or absent
  source 1..36: mol_type = other DNA
attggattaactttggtggaacctacttcgatnnat

SEQ ID NO: 138 - DNA, 33 bp, synthetic construct
  misc_feature 1..33: note = Synthetic polynucleotide
  misc_feature 30..31: note = n = any nucleotide, present or absent
  source 1..33: mol_type = other DNA
ctcccttctttcatccctacatcatgtccnnaa

SEQ ID NO: 139 - DNA, 27 bp, synthetic construct
  misc_feature 1..27: note = Synthetic polynucleotide
  misc_feature 24..25: note = n = any nucleotide, present or absent
  source 1..27: mol_type = other DNA
cggacttggctggggtagagcttnnaa

SEQ ID NO: 140 - DNA, 31 bp, synthetic construct
  misc_feature 1..31: note = Synthetic polynucleotide
  misc_feature 28..29: note = n = any nucleotide, present or absent
  source 1..31: mol_type = other DNA
gagctaagtaccaggtatgatgctcgcnnat

SEQ ID NO: 141 - DNA, 33 bp, synthetic construct
  misc_feature 1..33: note = Synthetic polynucleotide
  misc_feature 30..31: note = n = any nucleotide, present or absent
  source 1..33: mol_type = other DNA
agggaatgctctagacaaaacactgttccnnta

SEQ ID NO: 142 - DNA, 32 bp, synthetic construct
  misc_feature 1..32: note = Synthetic polynucleotide
  misc_feature 29..30: note = n = any nucleotide, present or absent
  source 1..32: mol_type = other DNA
tgctttgtgctagctcaaagactcacatnntt

SEQ ID NO: 143 - DNA, 38 bp, synthetic construct
  misc_feature 1..38: note = Synthetic polynucleotide
  misc_feature 35..36: note = n = any nucleotide, present or absent
  source 1..38: mol_type = other DNA
aattctggatcaaattaaatatgtcgcattctccnngt

SEQ ID NO: 144 - DNA, 39 bp, synthetic construct
  misc_feature 1..39: note = Synthetic polynucleotide
  misc_feature 36..37: note = n = any nucleotide, present or absent
  source 1..39: mol_type = other DNA
tgtactttctttttagccataagatgatttcccatnnat

SEQ ID NO: 145 - DNA, 28 bp, synthetic construct
  misc_feature 1..28: note = Synthetic polynucleotide
  misc_feature 25..26: note = n = any nucleotide, present or absent
  source 1..28: mol_type = other DNA
gcttgctttcccacaccactacctnnta

SEQ ID NO: 146 - DNA, 30 bp, synthetic construct
  misc_feature 1..30: note = Synthetic polynucleotide
  misc_feature 27..28: note = n = any nucleotide, present or absent
  source 1..30: mol_type = other DNA
ggtcaagtctgaggctgttgagcttannga

SEQ ID NO: 147 - DNA, 29 bp, synthetic construct
  misc_feature 1..29: note = Synthetic polynucleotide
  misc_feature 26..27: note = n = any nucleotide, present or absent
  source 1..29: mol_type = other DNA
ttcaggacgtgaaagcacgagaacgnnat

SEQ ID NO: 148 - DNA, 35 bp, synthetic construct
  misc_feature 1..35: note = Synthetic polynucleotide
  misc_feature 32..33: note = n = any nucleotide, present or absent
  source 1..35: mol_type = other DNA
atgtacaatttcaactggagtttccattgcanngt

SEQ ID NO: 149 - DNA, 30 bp, synthetic construct
  misc_feature 1..30: note = Synthetic polynucleotide
  misc_feature 27..28: note = n = any nucleotide, present or absent
  source 1..30: mol_type = other DNA
aaatagtgagaacgagcagctgcaggnnct

SEQ ID NO: 150 - DNA, 41 bp, synthetic construct
  misc_feature 1..41: note = Synthetic polynucleotide
  misc_feature 38..39: note = n = any nucleotide, present or absent
  source 1..41: mol_type = other DNA
aagaatataaaatgttagagaaccacatacaacgagcnnct

SEQ ID NO: 151 - DNA, 30 bp, synthetic construct
  misc_feature 1..30: note = Synthetic polynucleotide
  misc_feature 27..28: note = n = any nucleotide, present or absent
  source 1..30: mol_type = other DNA
aaccccagaacactagcagctaagggnnta

SEQ ID NO: 152 - DNA, 45 bp, synthetic construct
  misc_feature 1..45: note = Synthetic polynucleotide
  misc_feature 42..43: note = n = any nucleotide, present or absent
  source 1..45: mol_type = other DNA
ttttattagttgtgtaatccagttacttaactttaaaagccnnat

SEQ ID NO: 153 - DNA, 30 bp, synthetic construct
  misc_feature 1..30: note = Synthetic polynucleotide
  misc_feature 27..28: note = n = any nucleotide, present or absent
  source 1..30: mol_type = other DNA
gttctgaaaagagcctccactcctgtnntt

SEQ ID NO: 154 - DNA, 30 bp, synthetic construct
  misc_feature 1..30: note = Synthetic polynucleotide
  misc_feature 27..28: note = n = any nucleotide, present or absent
  source 1..30: mol_type = other DNA
cctccactccaccatggcacctattannaa

SEQ ID NO: 155 - DNA, 45 bp, synthetic construct
  misc_feature 1..45: note = Synthetic polynucleotide
  misc_feature 42..43: note = n = any nucleotide, present or absent
  source 1..45: mol_type = other DNA
gtataccacttaggctatagttattctaaactttgataaacnngt

SEQ ID NO: 156 - DNA, 39 bp, synthetic construct
  misc_feature 1..39: note = Synthetic polynucleotide
  misc_feature 36..37: note = n = any nucleotide, present or absent
  source 1..39: mol_type = other DNA
aggaatcattacaggaaaacatcgtttaaattggannaa

SEQ ID NO: 157 - DNA, 37 bp, synthetic construct
  misc_feature 1..37: note = Synthetic polynucleotide
  misc_feature 34..35: note = n = any nucleotide, present or absent
  source 1..37: mol_type = other DNA
ccatggtatattgtaagttgtaggtacatacccnnaa

SEQ ID NO: 158 - DNA, 37 bp, synthetic construct
  misc_feature 1..37: note = Synthetic polynucleotide
  misc_feature 34..35: note = n = any nucleotide, present or absent
  source 1..37: mol_type = other DNA
tttgatttgaataaaccagagaactcttctgagnntt

SEQ ID NO: 159 - DNA, 34 bp, synthetic construct
  misc_feature 1..34: note = Synthetic polynucleotide
  misc_feature 31..32: note = n = any nucleotide, present or absent
  source 1..34: mol_type = other DNA
tgttgctatcttgcttttagcatttagtgcnnaa

SEQ ID NO: 160 - DNA, 29 bp, synthetic construct
  misc_feature 1..29: note = Synthetic polynucleotide
  misc_feature 26..27: note = n = any nucleotide, present or absent
  source 1..29: mol_type = other DNA
tcagcgttgagtaataccgtctgccnnca

SEQ ID NO: 161 - DNA, 23 bp, synthetic construct
  misc_feature 1..23: note = Synthetic polynucleotide
  source 1..23: mol_type = other DNA
taagtggaaagaactggggtgtc

SEQ ID NO: 162 - DNA, 28 bp, synthetic construct
  misc_feature 1..28: note = Synthetic polynucleotide
  source 1..28: mol_type = other DNA
acatggttagatattagcctgacctatg

SEQ ID NO: 163 - DNA, 23 bp, synthetic construct
  misc_feature 1..23: note = Synthetic polynucleotide
  source 1..23: mol_type = other DNA
gcagccaagtgtgaaagtattga

SEQ ID NO: 164 - DNA, 25 bp, synthetic construct
  misc_feature 1..25: note = Synthetic polynucleotide
  source 1..25: mol_type = other DNA
tgatgttgagttgagacaggttaca

SEQ ID NO: 165 - DNA, 28 bp, synthetic construct
  misc_feature 1..28: note = Synthetic polynucleotide
  source 1..28: mol_type = other DNA
aaatgtagttctattatggtcagcacac

SEQ ID NO: 166 - DNA, 23 bp, synthetic construct
  misc_feature 1..23: note = Synthetic polynucleotide
  source 1..23: mol_type = other DNA
agtatccccaaaaggttgcagat

SEQ ID NO: 167 - DNA, 18 bp, synthetic construct
  misc_feature 1..18: note = Synthetic polynucleotide
  source 1..18: mol_type = other DNA
gtgcaagctggaggcact

SEQ ID NO: 168 - DNA, 29 bp, synthetic construct
  misc_feature 1..29: note = Synthetic polynucleotide
  source 1..29: mol_type = other DNA
acaggaaaagaaactaaaattgtaccctt

SEQ ID NO: 169 - DNA, 23 bp, synthetic construct
  misc_feature 1..23: note = Synthetic polynucleotide
  source 1..23: mol_type = other DNA
gaagccagatctcaaagtgtcct

SEQ ID NO: 170 - DNA, 27 bp, synthetic construct
  misc_feature 1..27: note = Synthetic polynucleotide
  source 1..27: mol_type = other DNA
gttattgggaatgctatgaaagagaca

SEQ ID NO: 171 - DNA, 29 bp, synthetic construct
  misc_feature 1..29: note = Synthetic polynucleotide
  source 1..29: mol_type = other DNA
agaactcatttccttatagctgaagaact

SEQ ID NO: 172 - DNA, 24 bp, synthetic construct
  misc_feature 1..24: note = Synthetic polynucleotide
  source 1..24: mol_type = other DNA
gcagacacttaggatgtttccagt

SEQ ID NO: 173 - DNA, 25 bp, synthetic construct
  misc_feature 1..25: note = Synthetic polynucleotide
  source 1..25: mol_type = other DNA
gagccttagttcctccatcagtaaa

SEQ ID NO: 174 - DNA, 27 bp, synthetic construct
  misc_feature 1..27: note = Synthetic polynucleotide
  source 1..27: mol_type = other DNA
aaatttacgttggtaattgggtcttgt

SEQ ID NO: 175 - DNA, 22 bp, synthetic construct
  misc_feature 1..22: note = Synthetic polynucleotide
  source 1..22: mol_type = other DNA
cacagaggtgacagaacacagt

SEQ ID NO: 176 - DNA, 24 bp, synthetic construct
  misc_feature 1..24: note = Synthetic polynucleotide
  source 1..24: mol_type = other DNA
tagttattcatggtgggaaggcaa

SEQ ID NO: 177 - DNA, 31 bp, synthetic construct
  misc_feature 1..31: note = Synthetic polynucleotide
  source 1..31: mol_type = other DNA
aaaagataatgttcttgtttatatgcccttg

SEQ ID NO: 178 - DNA, 27 bp, synthetic construct
  misc_feature 1..27: note = Synthetic polynucleotide
  source 1..27: mol_type = other DNA
tacagcaactcacaaactaatgactct

SEQ ID NO: 179 - DNA, 19 bp, synthetic construct
  misc_feature 1..19: note = Synthetic polynucleotide
  source 1..19: mol_type = other DNA
ggctgcgatgagacaggaa

SEQ ID NO: 180 - DNA, 23 bp, synthetic construct
  misc_feature 1..23: note = Synthetic polynucleotide
  source 1..23: mol_type = other DNA
aggcaccagaagtcatcagaatg

SEQ ID NO: 181 - DNA, 22 bp, synthetic construct
  misc_feature 1..22: note = Synthetic polynucleotide
  source 1..22: mol_type = other DNA
gaccaagcttttatgcaccaca

SEQ ID NO: 182 - DNA, 31 bp, synthetic construct
  misc_feature 1..31: note = Synthetic polynucleotide
  source 1..31: mol_type = other DNA
tgaaagatagcaatagatacataaaacacca

SEQ ID NO: 183 - DNA, 26 bp, synthetic construct
  misc_feature 1..26: note = Synthetic polynucleotide
  source 1..26: mol_type = other DNA
tgaaaccacatacacacaaattcact

SEQ ID NO: 184 - DNA, 23 bp, synthetic construct
  misc_feature 1..23: note = Synthetic polynucleotide
  source 1..23: mol_type = other DNA
cttcctgctgtgcatctgtaagt

SEQ ID NO: 185 - DNA, 24 bp, synthetic construct
  misc_feature 1..24: note = Synthetic polynucleotide
  source 1..24: mol_type = other DNA
aaaaagaagaaacggaaggcagag

SEQ ID NO: 186 - DNA, 22 bp, synthetic construct
  misc_feature 1..22: note = Synthetic polynucleotide
  source 1..22: mol_type = other DNA
tgctgccccaccctttattaac

SEQ ID NO: 187 - DNA, 23 bp, synthetic construct
  misc_feature 1..23: note = Synthetic polynucleotide
  source 1..23: mol_type = other DNA
cctattggaagaacctgccagaa

SEQ ID NO: 188 - DNA, 20 bp, synthetic construct
  misc_feature 1..20: note = Synthetic polynucleotide
  source 1..20: mol_type = other DNA
tgcaaaatgaagcacagccc

SEQ ID NO: 189 - DNA, 28 bp, synthetic construct
  misc_feature 1..28: note = Synthetic polynucleotide
  source 1..28: mol_type = other DNA
gcagatggaaaatacttgggaaaaaaat

SEQ ID NO: 190 - DNA, 33 bp, synthetic construct
  misc_feature 1..33: note = Synthetic polynucleotide
  source 1..33: mol_type = other DNA
gcaaaaattactataccgactttaataacgaaa

SEQ ID NO: 191 - DNA, 23 bp, synthetic construct
  misc_feature 1..23: note = Synthetic polynucleotide
  source 1..23: mol_type = other DNA
actcattgtaggctgaaccttgg

SEQ ID NO: 192 - DNA, 24 bp, synthetic construct
  misc_feature 1..24: note = Synthetic polynucleotide
  source 1..24: mol_type = other DNA
tgtattgagcatttagcacatgcc

SEQ ID NO: 193 - DNA, 24 bp, synthetic construct
  misc_feature 1..24: note = Synthetic polynucleotide
  source 1..24: mol_type = other DNA
caatttccaagacagaagcactcc

SEQ ID NO: 194 - DNA, 21 bp, synthetic construct
  misc_feature 1..21: note = Synthetic polynucleotide
  source 1..21: mol_type = other DNA
acttactgagcacatggcctg

SEQ ID NO: 195 - DNA, 21 bp, synthetic construct
  misc_feature 1..21: note = Synthetic polynucleotide
  source 1..21: mol_type = other DNA
ggagagggtgagaagttgcac

SEQ ID NO: 196 - DNA, 28 bp, synthetic construct
  misc_feature 1..28: note = Synthetic polynucleotide
  source 1..28: mol_type = other DNA
ggcaaagacatttttccaaggaagatat

SEQ ID NO: 197 - DNA, 18 bp, synthetic construct
  misc_feature 1..18: note = Synthetic polynucleotide
  source 1..18: mol_type = other DNA
cactgccagcttgtgcct

SEQ ID NO: 198 - DNA, 26 bp, synthetic construct
  misc_feature 1..26: note = Synthetic polynucleotide
  source 1..26: mol_type = other DNA
gccctaaatcctaaatgaaattggca

SEQ ID NO: 199 - DNA, 24 bp, synthetic construct
  misc_feature 1..24: note = Synthetic polynucleotide
  source 1..24: mol_type = other DNA
agaggagaaatagatgtagctgcc

SEQ ID NO: 200 - DNA, 28 bp, synthetic construct
  misc_feature 1..28: note = Synthetic polynucleotide
  source 1..28: mol_type = other DNA
aatccagtgacattctttaaactgtctt

SEQ ID NO: 201 - DNA, 22 bp, synthetic construct
  misc_feature 1..22: note = Synthetic polynucleotide
  source 1..22: mol_type = other DNA
gctgagctgtcacatcacttca

SEQ ID NO: 202 - DNA, 25 bp, synthetic construct
  misc_feature 1..25: note = Synthetic polynucleotide
  source 1..25: mol_type = other DNA
gctgtgtagcttggcaaattaacta

SEQ ID NO: 203 - DNA, 22 bp, synthetic construct
  misc_feature 1..22: note = Synthetic polynucleotide
  source 1..22: mol_type = other DNA
gcactcttgggtaacaggcttt

SEQ ID NO: 204 - DNA, 16 bp, synthetic construct
  misc_feature 1..16: note = Synthetic polynucleotide
  source 1..16: mol_type = other DNA
ccatgcccagcctggc

SEQ ID NO: 205 - DNA, 26 bp, synthetic construct
  misc_feature 1..26: note = Synthetic polynucleotide
  source 1..26: mol_type = other DNA
tggctcctcataagttatgcagattt

SEQ ID NO: 206 - DNA, 28 bp, synthetic construct
  misc_feature 1..28: note = Synthetic polynucleotide
  source 1..28: mol_type = other DNA
cagtaggattggctttatcaaagagatc

SEQ ID NO: 207 - DNA, 28 bp, synthetic construct
  misc_feature 1..28: note = Synthetic polynucleotide
  source 1..28: mol_type = other DNA
accataatgttttccatagaagatgcac

SEQ ID NO: 208 - DNA, 32 bp, synthetic construct
  misc_feature 1..32: note = Synthetic polynucleotide
  source 1..32: mol_type = other DNA
ggttctgtactgaagtaaaaatctcatactat

SEQ ID NO: 209 - DNA, 28 bp, synthetic construct
  misc_feature 1..28: note = Synthetic polynucleotide
  source 1..28: mol_type = other DNA
ggcaaagaaacatggcagaaatatcata

SEQ ID NO: 210 - DNA, 25 bp, synthetic construct
  misc_feature 1..25: note = Synthetic polynucleotide
  source 1..25: mol_type = other DNA
cctttggcattttggtcaagattgt

SEQ ID NO: 211 - DNA, 25 bp, synthetic construct
  misc_feature 1..25: note = Synthetic polynucleotide
  source 1..25: mol_type = other DNA
gggggaaaatggtttcttaggatga

SEQ ID NO: 212 - DNA, 31 bp, synthetic construct
  misc_feature 1..31: note = Synthetic polynucleotide
  source 1..31: mol_type = other DNA
ctcccatttttctaagacatttttttttctc

SEQ ID NO: 213 - DNA, 16 bp, synthetic construct
  misc_feature 1..16: note = Synthetic polynucleotide
  source 1..16: mol_type = other DNA
agcatgccgcccttgg

SEQ ID NO: 214 - DNA, 27 bp, synthetic construct
  misc_feature 1..27: note = Synthetic polynucleotide
  source 1..27: mol_type = other DNA
tcacaggtcaaaattatgagttcttcg

SEQ ID NO: 215 - DNA, 22 bp, synthetic construct
  misc_feature 1..22: note = Synthetic polynucleotide
  source 1..22: mol_type = other DNA
tgagagtgtgcaagtcacttgt

SEQ ID NO: 216 - DNA, 20 bp, synthetic construct
  misc_feature 1..20: note = Synthetic polynucleotide
  source 1..20: mol_type = other DNA
gcaggcagcatgtatcccag

SEQ ID NO: 217 - DNA, 31 bp, synthetic construct
  misc_feature 1..31: note = Synthetic polynucleotide
  source 1..31: mol_type = other DNA
gtttaatggacagtagatgctaaattctaga

SEQ ID NO: 218 - DNA, 20 bp, synthetic construct
  misc_feature 1..20: note = Synthetic polynucleotide
  source 1..20: mol_type = other DNA
cgccatagttagccgcttcc

SEQ ID NO: 219 - DNA, 20 bp, synthetic construct
  misc_feature 1..20: note = Synthetic polynucleotide
  source 1..20: mol_type = other DNA
tgagcctcggtctctacctg

SEQ ID NO: 220 - DNA, 20 bp, synthetic construct
  misc_feature 1..20: note = Synthetic polynucleotide
  source 1..20: mol_type = other DNA
cctttaaggcccagcaactg

SEQ ID NO: 221 - DNA, 23 bp, synthetic construct
  misc_feature 1..23: note = Synthetic polynucleotide
  source 1..23: mol_type = other DNA
gggtgacctttcccttttgatga

SEQ ID NO: 222 - DNA, 26 bp, synthetic construct
  misc_feature 1..26: note = Synthetic polynucleotide
  source 1..26: mol_type = other DNA
tgtgtgtgaaagcactttataaacca

SEQ ID NO: 223 - DNA, 34 bp, synthetic construct
  misc_feature 1..34: note = Synthetic polynucleotide
  source 1..34: mol_type = other DNA
ctatcctcagaattttccattgatactagaaata

SEQ ID NO: 224 - DNA, 23 bp, synthetic construct
  misc_feature 1..23: note = Synthetic polynucleotide
  source 1..23: mol_type = other DNA
gagtgtctcccaaacaaggatca

SEQ ID NO: 225 - DNA, 22 bp, synthetic construct
  misc_feature 1..22: note = Synthetic polynucleotide
  source 1..22: mol_type = other DNA
acagccatcagatatccagcag

SEQ ID NO: 226 - DNA, 25 bp, synthetic construct
  misc_feature 1..25: note = Synthetic polynucleotide
  source 1..25: mol_type = other DNA
acttcgagaattgactctaagtggt

SEQ ID NO: 227 - DNA, 29 bp, synthetic construct
  misc_feature 1..29: note = Synthetic polynucleotide
  source 1..29: mol_type = other DNA
aatttagcttccttgaggatagaagtaac

SEQ ID NO: 228 - DNA, 17 bp, synthetic construct
  misc_feature 1..17: note = Synthetic polynucleotide
  source 1..17: mol_type = other DNA
cccggccacccatacag

SEQ ID NO: 229 - DNA, 31 bp, synthetic construct
  misc_feature 1..31: note = Synthetic polynucleotide
  source 1..31: mol_type = other DNA
gaaaactaccttaaactatgtgagaaagaac

SEQ ID NO: 230 - DNA, 28 bp, synthetic construct
  misc_feature 1..28: note = Synthetic polynucleotide
  source 1..28: mol_type = other DNA
accctcactaatctttttctgtttgttt

SEQ ID NO: 231 - DNA, 24 bp, synthetic construct
  misc_feature 1..24: note = Synthetic polynucleotide
  source 1..24: mol_type = other DNA
gtttttctcccagctgtaaaagca

SEQ ID NO: 232 - DNA, 31 bp, synthetic construct
  misc_feature 1..31: note = Synthetic polynucleotide
  source 1..31: mol_type = other DNA
gctttagtttctttgcatattttctgcaata

SEQ ID NO: 233 - DNA, 24 bp, synthetic construct
  misc_feature 1..24: note = Synthetic polynucleotide
  source 1..24: mol_type = other DNA
agctgatctgcaaggtctatttga

SEQ ID NO: 234 - DNA, 21 bp, synthetic construct
  misc_feature 1..21: note = Synthetic polynucleotide
  source 1..21: mol_type = other DNA
tgggctcaagtgatccaccta

SEQ ID NO: 235 - DNA, 25 bp, synthetic construct
  misc_feature 1..25: note = Synthetic polynucleotide
  source 1..25: mol_type = other DNA
gtaaagagaagggctaccaggatta

SEQ ID NO: 236 - DNA, 23 bp, synthetic construct
  misc_feature 1..23: note = Synthetic polynucleotide
  source 1..23: mol_type = other DNA
ccctatgcctgggatacttcctt

SEQ ID NO: 237 - DNA, 31 bp, synthetic construct
  misc_feature 1..31: note = Synthetic polynucleotide
  source 1..31: mol_type = other DNA
acaaatctttcatttgtctaaggtatcaact

SEQ ID NO: 238 - DNA, 26 bp, synthetic construct
  misc_feature 1..26: note = Synthetic polynucleotide
  source 1..26: mol_type = other DNA
agtgtcttgcattttcaagtattcct

SEQ ID NO: 239 - DNA, 29 bp, synthetic construct
  misc_feature 1..29: note = Synthetic polynucleotide
  source 1..29: mol_type = other DNA
cctaggttatttgctgttctctttcatta

SEQ ID NO: 240 - DNA, 28 bp, synthetic construct
  misc_feature 1..28: note = Synthetic polynucleotide
  source 1..28: mol_type = other DNA
gcttgcatatagacctacaaataccact

SEQ ID NO: 241 - DNA, 18 bp, synthetic construct
  misc_feature 1..18: note = Synthetic polynucleotide
  source 1..18: mol_type = other DNA
ggcaccatgcatccagcc

SEQ ID NO: 242 - DNA, 21 bp, synthetic construct
  misc_feature 1..21: note = Synthetic polynucleotide
  source 1..21: mol_type = other DNA
gtccaagagtggaggattggg

SEQ ID NO: 243 - DNA, 26 bp, synthetic construct
  misc_feature 1..26: note = Synthetic polynucleotide
  source 1..26: mol_type = other DNA
ggaaatcgtacagttccaaagtacaa

SEQ ID NO: 244 - DNA, 24 bp, synthetic construct
  misc_feature 1..24: note = Synthetic polynucleotide
  source 1..24: mol_type = other DNA
acagcaacagaaactacccaaaag

SEQ ID NO: 245 - DNA, 23 bp, synthetic construct
  misc_feature 1..23: note = Synthetic polynucleotide
  source 1..23: mol_type = other DNA
caagcatcttctccttcctctct

SEQ ID NO: 246 - DNA, 25 bp, synthetic construct
  misc_feature 1..25: note = Synthetic polynucleotide
  source 1..25: mol_type = other DNA
cctagtccttaaccactccttacag

SEQ ID NO: 247 - DNA, 24 bp, synthetic construct
  misc_feature 1..24: note = Synthetic polynucleotide
  source 1..24: mol_type = other DNA
tccaaactggaaatggctgtatct

SEQ ID NO: 248 - DNA, 33 bp, synthetic construct
  misc_feature 1..33: note = Synthetic polynucleotide
  source 1..33: mol_type = other DNA
gctttctatttatttaaaagaaagtgaagtccc

SEQ ID NO: 249 - DNA, 24 bp, synthetic construct
  misc_feature 1..24: note = Synthetic polynucleotide
  source 1..24: mol_type = other DNA
tttccccccttctctcttcttttt

SEQ ID NO: 250 - DNA, 23 bp, synthetic construct
  misc_feature 1..23: note = Synthetic polynucleotide
  source 1..23: mol_type = other DNA
cagcagtacactgaacagaatcc

SEQ ID NO: 251 - DNA, 57 bp, synthetic construct
  misc_feature 1..57: note = Synthetic polynucleotide
  source 1..57: mol_type = other DNA
agggaccaggtaaatatttaccacgtcttggtctttattttaccgtctatatacaag

SEQ ID NO: 252 - DNA, 57 bp, synthetic construct
  misc_feature 1..57: note = Synthetic polynucleotide
  source 1..57: mol_type = other DNA
agggaccaggtaaatatttaccacctcttggtgtttattttaccgtctatatacaag